(19) United States
(12) Patent Application Publication
(10) Pub. No.: US 2014/0065070 A1
(43) Pub. Date: Mar. 6, 2014

(54) METHODS OF PREPARING TRIAZOLE-CONTAINING RADIOIODINATED COMPOUNDS

(71) Applicants: John Valliant, Ancaster (CA); Alla Darwish, Kitchener (CA); Stephanie Rathmann, Hamilton (CA); Afaf Genady, Hamilton (CA)

(72) Inventors: John Valliant, Ancaster (CA); Alla Darwish, Kitchener (CA); Stephanie Rathmann, Hamilton (CA); Afaf Genady, Hamilton (CA)

(73) Assignee: MCMASTER UNIVERSITY, Hamilton (CA)

(21) Appl. No.: 14/012,502

(22) Filed: Aug. 28, 2013

Related U.S. Application Data
(60) Provisional application No. 61/693,921, filed on Aug. 28, 2012.

(51) Int. Cl. A61K 51/04

(52) U.S. Cl. CPC A61K 51/0453; A61K 51/0455
USPC 424/1.85; 548/255; 546/210; 548/103

(57) ABSTRACT

The present application relates to methods of preparing radiohalogenated compounds, to compounds useful in such methods and to radiohalogenated compounds useful for imaging and/or therapy. In particular, the present application relates to methods of preparing radiohalogenated compounds of Formula I, to compounds useful in such methods and to radiohalogenated compounds of Formula I:

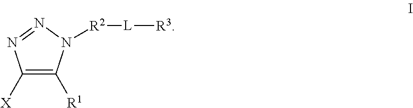

### CROSS-REFERENCE TO RELATED APPLICATIONS

The present application claims the benefit of priority from co-pending U.S. provisional application No. 61/693,921 filed on Aug. 28, 2012, the contents of which are incorporated herein by reference in their entirety.

### FIELD

The present application relates to methods of preparing triazole-containing radiohalogenated compounds, to compounds useful in such methods and radiohalogenated compounds that are useful for imaging and/or therapy.

### BACKGROUND

Molecular imaging of cancer using isotopes of iodine is becoming increasingly attractive because of the ability to develop isostructural theranostics: agents that can be used for both diagnosis and treatment.¹ By simply changing the isotope of iodine, it is possible to convert a useful PET or SPECT imaging agent (based on ¹²⁴I or ¹³¹I/¹²³I respectively) into a targeted therapeutic compound (based on ¹²⁵I or ¹³¹I) without altering the structure of the molecule. However, the development of these types of agents is hindered by the limited number of iodine-containing prosthetic groups that are synthetically accessible, resistant to catabolism and/or that can penetrate and be retained in tumours when appended to the appropriate targeting vector.² The focus to date has largely been on iodobenzene-derived prosthetic groups, which do not meet these criteria and they are typically lipophilic thereby promoting non-specific binding particularly when appended to small molecules.^{2c}

Radioiodinated heterocycles are an alternative to benzene-derived prosthetic groups. For example, Årstad et al.³ reported the preparation of trifunctional reagents for multiscale imaging using both optical and nuclear techniques. In the studies reported by Årstad et al., ¹²⁵I triazoles were formed in situ when a click reaction was performed between an alkyne-derived fluorophore and an azide-derived active ester in the presence of NaI.

PCT Publication No. WO 2011/020907 describes the preparation of ¹²³I-labeled acetylene which could then be “clicked” to form labeled heterocycles.

### SUMMARY

Methods for preparing a prosthetic group referred to herein as the triazole appending agent (TAAG) which can be used to make radiohalogen-based molecular imaging and targeted therapy agents have been developed in the present studies. The TAAG group can be appended to suitable targeting vectors to prepare radiohalogenated, for example radioiodinated, probes that, for example, clear rapidly from non-target tissues and/or are able to penetrate tumor cells. Functionalized-TAAG derivatives have also been prepared which provide a second site useful for further derivatization, such as biomolecule or fluorophore derivatization.

Accordingly, the present application includes a method of preparing a radiohalogenated compound of Formula I:

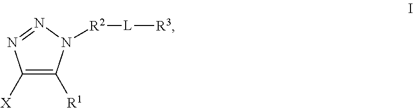

wherein

R¹ is selected from:
(i) H;
(ii) cyano:
(iii) OR⁴;
(iv) NR⁴R⁵;
(v) substituted or unsubstituted C₁₋₆alkyl;
(vi) substituted or unsubstituted C₂₋₆alkenyl;
(vii) substituted or unsubstituted C₃₋₈cycloalkyl;
(viii) substituted or unsubstituted C₃₋₈cycloalkenyl;
(ix) substituted or unsubstituted C₂₋₈heterocycloalkyl;
(x) substituted or unsubstituted C₆₋₁₄aryl; and
(xi) substituted or unsubstituted heteroaryl,
   wherein the substituents for C₁₋₆alkyl, C₂₋₆alkenyl, C₃₋₈cycloalkyl, C₃₋₈cycloalkenyl, C₂₋₈heterocycloalkyl, C₆₋₁₄aryl and heteroaryl are selected from F, Cl, Br, I, cyano, oxo, nitro, OR⁴, NR⁴R⁵, C(O)OR⁴, C(O)N⁴R⁵, C₁₋₄alkyl, C₃₋₈cycloalkyl, C₂₋₈heterocycloalkyl, C₆₋₁₀aryl and an immunogenic moiety;

R² is C₁₋₄alkylene;

R₃ is H, C₁₋₄alkyl or a targeting vector;

R⁴ and R⁵ are each independently selected from H, PG, C₁₋₆alkyl, C₂₋₆alkenyl, C₃₋₈cycloalkyl, C₃₋₈cycloalkenyl, C₆₋₁₀aryl, a targeting vector, a fluorophore and an immunogenic moiety; or R⁴ and R⁵ together form PG;

L is an amide linkage or an ester linkage; and

X is a radioisotope of a halogen,

comprising reacting a compound of Formula II:

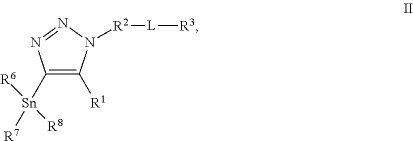

wherein

L, R¹, R² and R³ are as defined for the compound of Formula I; and

R⁶, R⁷ and R⁸ are each independently C₁₋₁₀alkyl or C₁₋₁₀alkyl substituted with one or more F;

with a radiohalogenating agent under conditions to obtain the compound of Formula I.

In an embodiment, the radiohalogenated compound of Formula I is a radiohalogenated compound of Formula I(a):

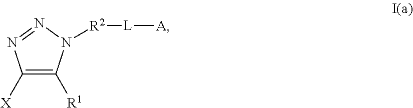

wherein

R¹, R², L and X are as defined for the compound of Formula I of claim 1; and

A is a targeting vector,

and the method comprises reacting a compound of Formula II(a):

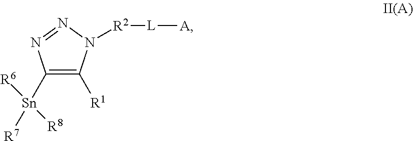

wherein

R¹, R², L and A are as defined for the compound of Formula I(a); and

R⁶, R⁷ and R⁸ are each independently C₁₋₁₀alkyl or C₁₋₁₀alkyl substituted with one or more F,

with a radiohalogenating agent under conditions to obtain the compound of Formula I(a).

In another embodiment, the method further comprises preparing the compound of Formula II(a) by steps comprising:

(a) reacting a compound of Formula III:

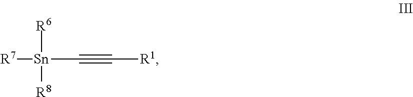

wherein

R¹ is as defined for the compound of Formula I in claim 1; and

R⁶, R⁷ and R⁸ are each independently C₁₋₁₀alkyl or C₁₋₁₀alkyl substituted with one or more F,

with a compound of Formula IV:

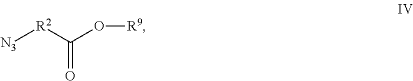

wherein

R² is C₁₋₄alkylene; and

R⁹ is H, C₁₋₄alkyl or an activating group,

under conditions to obtain a compound of Formula V:

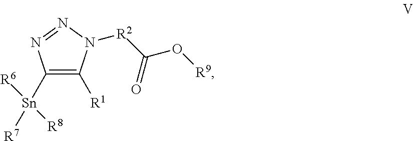

wherein

R¹ is as defined in the compound of Formula I of claim 1;

R² is C₁₋₄alkylene,

R⁶, R⁷ and R⁸ are each independently C₁₋₁₀alkyl or C₁₋₁₀alkyl substituted with one or more F; and

R⁹ is H, C₁₋₄alkyl or an activating group; and

(b) reacting the compound of Formula V with a compound of Formula VI:

H—R¹⁰-A VI,

wherein

R¹⁰ is O or NH; and

A is a targeting vector,

under conditions to obtain the compound of Formula II(a).

In an alternate embodiment, the method further comprises preparing the compound of Formula II(a) by steps comprising:

(a) reacting a compound of Formula VI:

H—R¹⁰-A VI,

wherein

R¹⁰ is O or NH; and

A is a targeting vector,

with a compound of Formula IV:

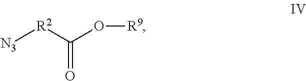

wherein

R² is C₁₋₄alkylene; and

R⁹ is H, C₁₋₄alkyl or an activating group,

under conditions to obtain a compound of Formula VII:

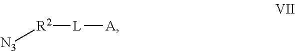

wherein

R² is C₁₋₄alkylene;

L is —C(O)O— or —C(O)NH—; and

A is a targeting vector; and

(b) reacting the compound of Formula VII with a compound of Formula III:

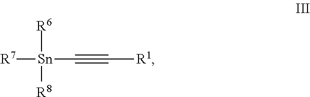

wherein

R¹ is as defined for the compound of Formula I of claim 1; and

R⁶, R⁷ and R⁸ are each independently C₁₋₁₀alkyl or C₁₋₁₀alkyl substituted with one or more F,

under conditions to obtain the compound of Formula II(a).

In an embodiment, the targeting vector targets cancer. In another embodiment, the cancer is prostate cancer or melanoma.

In an embodiment, L and A together have the structure:

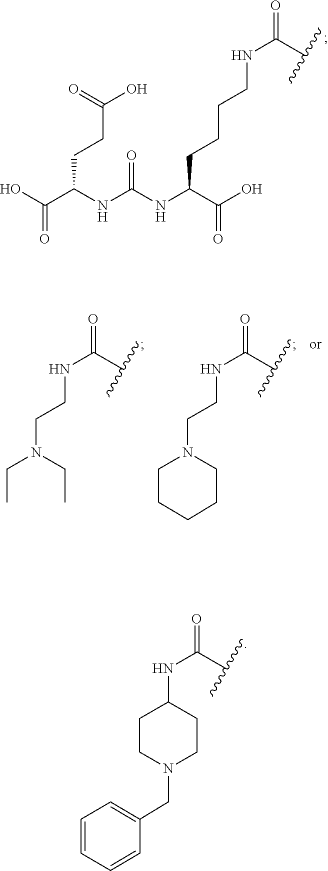 [SMILES: *C(=O)NCCCC[C@H](NC(=O)N[C@@H](CCC(=O)O)C(=O)O)C(=O)O ; *C(=O)NCCN(CC)CC ; *C(=O)NCCN1CCCCC1 ; *C(=O)NC1CCN(Cc2ccccc2)CC1]

In an embodiment, R¹ is selected from H, —CH₂NH₂, —CH₂NH-2,4-dinitrophenyl and phenyl. In another embodiment, R¹ is H. In a further embodiment, R¹ is —CH₂NH-2,4-dinitrophenyl.

In an embodiment, R² is —CH₂—. In another embodiment, R⁶, R⁷ and R⁸ are all n-Bu or are all (CH₂)₂(CF₂)₅CF₃. In a further embodiment, R⁹ is CH₃.

In an embodiment, the radiohalogenating agent is a radioiodinating agent. In another embodiment, the radioiodinating agent comprises I₂ or NaI, wherein I is a radioisotope of iodine. In a further embodiment, the radioisotope of iodine is ¹²³I, ¹²⁴I, ¹²⁵I or ¹³¹I.

The present application also includes a radiohalogenated compound of Formula I:

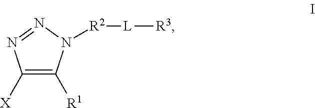

wherein

R¹ is selected from:
(i) H;
(ii) cyano;
(iii) OR⁴;
(iv) NR⁴R⁵;
(v) substituted or unsubstituted C₁₋₆alkyl;
(vi) substituted or unsubstituted C₂₋₆alkenyl;
(vii) substituted or unsubstituted C₃₋₈cycloalkyl;
(viii) substituted or unsubstituted C₃₋₈cycloalkenyl;
(ix) substituted or unsubstituted C₂₋₈heterocycloalkyl;
(x) substituted or unsubstituted C₆₋₁₄aryl; and
(xi) substituted or unsubstituted heteroaryl,
   wherein the substituents for C₁₋₆alkyl, C₂₋₆alkenyl, C₃₋₈cycloalkyl, C₃₋₈cycloalkenyl, C₂₋₈heterocycloalkyl, C₆₋₁₄aryl and heteroaryl are selected from F, Cl, Br, I, cyano, oxo, nitro, OR⁴, NR⁴R⁵, C(O)OR⁴, C(O)N⁴R⁵, C₁₋₄alkyl, C₃₋₈cycloalkyl, C₂₋₈heterocycloalkyl, C₆₋₁₀aryl and an immunogenic moiety;

R² is C₁₋₄alkylene;

R³ is H, C₁₋₄alkyl or a targeting vector;

R⁴ and R⁵ are each independently selected from H, PG, C₁₋₆alkyl, C₂₋₆alkenyl, C₃₋₈cycloalkyl, C₃₋₈cycloalkenyl, C₆₋₁₀aryl, a targeting vector, a fluorophore and an immunogenic moiety; or R⁴ and R⁵ together form PG;

L is an amide linkage or an ester linkage; and

X is a radioisotope of a halogen.

The present application further includes a compound of Formula II:

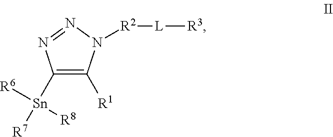

wherein

R¹ is selected from:
(i) H;
(ii) cyano;
(iii) OR⁴;
(iv) NR⁴R⁵;
(v) substituted or unsubstituted C₁₋₆alkyl;
(vi) substituted or unsubstituted C₂₋₆alkenyl;
(vii) substituted or unsubstituted C₃₋₈cycloalkyl;
(viii) substituted or unsubstituted C₃₋₈cycloalkenyl;
(ix) substituted or unsubstituted C₂₋₈heterocycloalkyl;
(x) substituted or unsubstituted C₆₋₁₄aryl; and
(xi) substituted or unsubstituted heteroaryl,
   wherein the substituents for C₁₋₆alkyl, C₂₋₆alkenyl, C₃₋₈cycloalkyl, C₃₋₈cycloalkenyl, C₂₋₈heterocycloalkyl, C₆₋₁₄aryl and heteroaryl are selected from F, Cl, Br, I, cyano, oxo, nitro, OR⁴, NR⁴R⁵, C(O)OR⁴, C(O)N⁴R⁵, C₃₋₈cycloalkyl, C₂₋₈heterocycloalkyl, C₆₋₁₀aryl and an immunogenic moiety;

R² is C₁₋₄alkylene;

R⁴ and R⁵ are each independently selected from H, PG, C₁₋₆alkyl, C₂₋₆alkenyl, C₃₋₈cycloalkyl, C₃₋₈cycloalkenyl, C₆₋₁₀aryl, a targeting vector, a fluorophore and an immunogenic moiety; or R⁴ and R⁵ together form PG;

L is an amide linkage or an ester linkage;

R⁶, R⁷ and R⁸ are each independently C₁₋₁₀alkyl or C₁₋₁₀alkyl substituted with one or more F; and

R³ is H, C₁₋₄alkyl or a targeting vector.

The present application also includes a composition comprising a compound of the application and a carrier.

The present application further includes a use of a compound of Formula II:

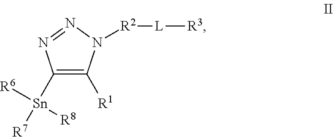

as defined herein for the preparation of a radiohalogenated compound.

Other features and advantages of the present application will become apparent from the following detailed description. It should be understood, however, that the detailed description and the specific examples while indicating embodiments of the application are given by way of illustration only, since various changes and modifications within the spirit and scope of the application will become apparent to those skilled in the art from this detailed description.

### BRIEF DESCRIPTION OF THE DRAWINGS

The present application will now be described in greater detail with reference to the drawings, in which:

FIG. 1 shows an ORTEP (Oak Ridge Thermal Ellipsoid Plot) representation (50% thermal probability ellipsoids) for ¹²⁷I-TAAG (2-(4-iodo-1H-1,2,3-triazol-1-yl)acetic acid).

FIG. 2 shows in vivo biodistribution of ¹²³I-TAAG in normal Balb/c mice. Mice were injected with ˜1.3 MBq of test article and sacrificed at various time points. Data expressed as % ID/g.

FIG. 3 shows an exemplary fused scintigraphic-CT image (23 h post-injection; 37 MBq of ¹²³I-TAAG-PSMA administered via the tail vein) of NCr nude mice containing an LNCap tumor.

FIG. 4 shows exemplary SPECT-CT images of LNCaP xenografts (indicated by arrows) administered ¹²³I-TAAG-PSMA (˜37 MBq). Mouse 1 was imaged at 2 (A) and 6 (C) hours post injection. Mouse 2 was imaged at 4 (B) and 23 (D) hours post injection. The tumor is highlighted by the arrows.

FIG. 5 shows biodistribution of ¹²³I-TAAG-PSMA in LNCaP xenograft mice. Mice were injected with ˜0.6 MBq of ¹²³I-TAAG-PSMA in the presence or absence of PMPA block (10 mg/kg) and sacrificed at various time points. Data expressed as % ID/g.

FIG. 6 shows a graph summarizing the results of ¹²⁵I-amino-TAAG-PSMA competition binding.

### DETAILED DESCRIPTION

### I. Definitions

Unless otherwise indicated, the definitions and embodiments described in this and other sections are intended to be applicable to all embodiments and aspects of the application herein described for which they are suitable as would be understood by a person skilled in the art.

The term “compound of the present application” or “compound of the application” as used herein refers to a compound of Formula I or II.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

The term “suitable” as used herein means that the selection of the particular compound or conditions would depend on the specific synthetic manipulation to be performed, and the identity of the species to be transformed, but the selection would be well within the skill of a person trained in the art. All method steps described herein are to be conducted under conditions sufficient to provide the desired product. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

In embodiments of the present application, the compounds described herein have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (e.g. less than 20%, suitably less than 10%, more suitably less than 5%) of compounds of the present application having alternate stereochemistry.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The expression “proceed to a sufficient extent” as used herein with reference to the reactions or method steps disclosed herein means that the reactions or method steps proceed to an extent that conversion of the starting material or substrate to product is maximized. Conversion may be maximized when greater than about 5, 10, 15, 20, 25, 30, 35, 40, 45, 50, 55, 60, 65, 70, 75, 80, 85, 90, 95 or 100% of the starting material or substrate is converted to product.

The term “acyl” as used herein means straight or branched chain, saturated acyl groups. The term C₁₋₆acyl means an acyl group having 1, 2, 3, 4, 5 or 6 carbon atoms.

The term “alkyl” as used herein means straight or branched chain, saturated alkyl groups. The term C₁₋₆alkyl means an alkyl group having 1, 2, 3, 4, 5 or 6 carbon atoms.

The term “alkylene” as used herein means straight or branched chain, saturated alkylene group, that is, a saturated carbon chain that contains substituents on two of its ends. The term C₁₋₄alkylene means an alkylene group having 1, 2, 3 or 4 carbon atoms.

The term “alkenyl” as used herein means straight or branched chain, unsaturated alkenyl groups. For example, the term C₂₋₆alkenyl means an alkenyl group having 2, 3, 4, 5 or 6 carbon atoms and at least one double bond, for example 1-3, 1-2 or 1 double bond.

The term “cycloalkyl” as used herein means saturated alkyl groups having at least one cyclic ring. For example, the term C₃₋₈cycloalkyl means a cycloalkyl group having 3, 4, 5, 6, 7 or 8 carbon atoms.

The term “cycloalkenyl” as used herein means cyclic, unsaturated alkenyl groups. For example, the term C₃₋₈cycloalkenyl means a cycloalkenyl group having 3, 4, 5, 6, 7 or 8 carbon atoms and at least one double bond.

The term “heterocycloalkyl” as used herein refers to a non-aromatic ring-containing group having one or more multivalent heteroatoms independently selected from the group consisting of N, O and S as a part of the ring structure. For example, the term C₂₋₈heterocycloalkyl means a heterocycloalkyl group having 2, 3, 4, 5, 6, 7 or 8 carbon atoms and at least one multivalent heteroatom selected from the group consisting of N, O and S as a part of the ring structure.

The term “aryl” as used herein refers to cyclic groups that contain at least one aromatic ring. In an embodiment of the application, the aryl group contains from 6, 9, 10 or 14 atoms, such as phenyl, naphthyl, indanyl or anthracenyl.

The term “heteroaryl” as used herein means a monocyclic ring or a polycyclic ring system containing 5, 6, 7, 8, 9, 10, 11, 12, 13, 14, 15, 16, 17, 18, 19 or 20 atoms, of which one or more, for example 1 to 8, 1 to 6, 1 to 5, or 1 to 4, of the atoms are a heteromoiety selected from O, S, NH and NC₁₋₆alkyl, with the remaining atoms being C, CH or CH₂, said ring system containing at least one aromatic ring. Examples of heteroaryl groups include, but are not limited to furanyl, thiophenyl, pyrrolyl, imidazolyl, pyrazolyl, triazolyl, oxazolyl, isoxazolyl, thiazolyl, isothiazolyl, oxadiazolyl, tetrazolyl, oxatriazolyl, isoxazinyl, pyridinyl, pyridazinyl, pyrimidinyl, pyrazinyl, triazinyl, benzofuranyl, isobenzofuranyl, benzothiophenyl, indolyl, isoindolyl, quinolinyl, isoquinolinyl, benzodiazinyl, pyridopyridinyl, acridinyl, xanthenyl and the like.

The term “oxo” as used herein refers to the group “═O”.

The term “nitro” as used herein refers to an NO₂ group.

The term “amino” as used herein refers to an NH₂ group or a protected form thereof.

The term “halo” as used herein refers to a halogen atom and includes F, Cl, Br, I and At.

The term “targeting vector” as used herein means a moiety which is taken up and retained in particular site of a subject such as a biological structure for example an organ or tissue or a pathological structure for example a tumor, with little or no accumulation and/or retention in non-target sites over a particular time period. In an embodiment, the targeting vector also is a moiety that is an inhibitor of a protein, for example a protein that is overexpressed in a disease, disorder or condition such as cancer. In another embodiment, the targeting vector is an antibody. Targeting vectors are known and the selection of a suitable targeting vector for a particular imaging or therapeutic use can be made by a person skilled in the art. Targeting vectors include, but are not limited to small molecules such as enzyme inhibitors or pharmaceutical-like compounds, peptides, proteins, nucleic acids or analogues or derivatives thereof, dendrimers, polymers and antibodies or fragments thereof.

The term “fluorophore” as used herein refers to a chemical moiety that is fluorescent and that can re-emit light upon light excitation. Fluorophores are known and the selection of a suitable fluorophore can be made by a person skilled in the art. Examples of fluorophores include, but are not limited to fluorescein and derivatives thereof, cyanine dyes, metal-based fluorophores, boron-dipyrromethene (BODIPY) dyes, sulforhodamine 101 acid chloride (Texas Red), Alexa Fluor™ dyes and rhodamine dyes.

The term “immunogenic moiety” as used herein refers to a moiety which induces an immune response in a subject. For example, the immunogenic moiety can be a moiety for which the subject has antibodies against. In an embodiment, the immunogenic moiety is a hapten such as a dinitrophenyl group.

The term “subject” as used herein includes all members of the animal kingdom including mammals, and suitably refers to humans.

The term “radiohalogenating agent” as used herein refers to a reagent that destannylates and radiohalogenates a compound comprising a tin-substituted triazole under conditions to obtain the corresponding radiohalogenated compound. In an embodiment of the present application, the radiohalogenating agent is a radioiodinating agent that destannylates and radioiodinates a compound comprising a tin-substituted triazole under conditions to obtain the corresponding radioiodinated compound. The selection of a suitable radiohalogenating agent such as a radioiodinating agent can be made by a person skilled in the art. In an embodiment, the radiohalogenating agent comprises X₂ or MX, wherein M is a cation such as an alkali metal cation or an organic cation such as a quaternary amine, for example [NMe₄]⁺ and X is a radioisotope of a halogen. In another embodiment, the radiohalogenating agent comprises NaX, wherein X is a radioisotope of a halogen. In a further embodiment, the radioiodinating agent comprises and iodide (I⁻) salt, wherein I⁻ is a radioisotope of iodine. In an embodiment the iodide salt is NaI.

The term “triazole derivative” as used herein refers to a compound or group which comprises a 1,2,3-triazole moiety.

The term “azide precursor” as used herein in reference to a triazole derivative refers to a compound comprising an azide functional group that can react with a suitable alkyne to prepare a triazole derivative. In an embodiment of the present application, the alkyne is a stannylalkyne. The reaction between an azide precursor and a stannylalkyne is useful, for example as it can be carried out in the absence of a catalyst and is regiospecific. The reaction between an azide precursor and an alkyne other than a stannylalkyne generally requires the presence of a catalyst such as a copper catalyst, for example copper(I) iodide.

The term “activating group” as used herein means a group that is obtained by reaction of a carboxylic acid with a suitable carboxylic acid activating reagent. Activating groups, along with the oxygen to which they are coupled can be displaced, for example upon reaction with a nucleophile such as a hydroxyl or NH group. Carboxylic acid activating reagents are well known in the art and include, for example, well known peptide coupling reagents such as dicyclohexylcarbodiimide (DCC), diisopropylcarbodiimide, hydroxybenzotriazole (HOBT), (Benzotriazol-1-yloxy)tris(dimethylamino)phosphonium hexafluorophosphate (BOP), [N,N,N′,N′-tetramethyl-O-(7-azabenzotriazol-1-yl)uronium hexafluorophosphate (HATU) and the like.

The term “leaving group” or “LG” as used herein refers to a group that is readily displaceable by a nucleophile, for example, under nucleophilic substitution reaction conditions. Examples of suitable leaving groups include, but are not limited to, halo, Ms, Ts, Ns, Tf, C₁₋₆acyl, and the like.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas). Examples of suitable protecting groups include, but are not limited to t-Boc, Ac, Ts, Ms, silyl ethers such as TMS, TBDMS, TBDPS, Tf, Ns, Bn, Fmoc, benzoyl, dimethoxytrityl, methoxyethoxymethyl ether, methoxymethyl ether, pivaloyl, p-methoxybenzyl ether, tetrahydropyranyl, trityl, ethoxyethyl ethers, carbobenzyloxy, benzoyl, phthalimide, t-butyl and the like.

t-Boc as used herein refers to the group t-butyloxycarbonyl.

Ac as used herein refers to the group acetyl.

Ts (tosyl) as used herein refers to the group p-toluenesulfonyl.

Ms as used herein refers to the group methanesulfonyl.

TMS as used herein refers to the group trimethylsilyl.

TBDMS as used herein refers to the group t-butyldimethylsilyl.

TBDPS as used herein refers to the group t-butyldiphenylsilyl.

Tf as used herein refers to the group trifluoromethanesulfonyl.

Ns as used herein refers to the group naphthalene sulphonyl.

Bn as used herein refers to the group benzyl.

Fmoc as used herein refers to the group fluorenylmethoxycarbonyl.

Cbz as used herein refers to the group carboxybenzyl.

TFA as used herein refers to trifluoroacetic acid.

DCM as used herein refers to dichloromethane.

ACN as used herein refers to acetonitrile.

AcOH as used herein refers to acetic acid.

DIPEA as used herein refers to N,N-diisopropylethylamine.

THF as used herein refers to tetrahydrofuran.

MeOH as used herein refers to methanol.

The term I-TAAG as used herein refers to a compound having the following structure:

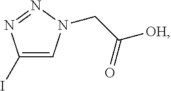

wherein a superscript before the I in the term I-TAAG indicates the isotope of iodine in the compound. For example, the term ¹²³I-TAAG refers to a compound having the above structure, wherein the I is ¹²³I.

The term I-TAAG-PSMA as used herein refers to a compound having the following structure:

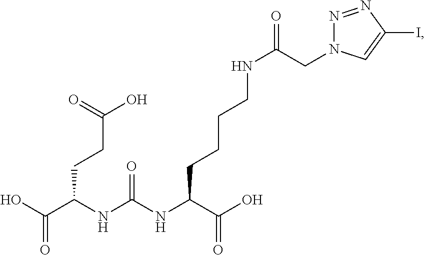 [SMILES: O=C(O)CC[C@H](NC(=O)N[C@@H](CCCCNC(=O)Cn1cc(I)nn1)C(=O)O)C(=O)O]

wherein a superscript before the I in the term I-TAAG-PSMA indicates the isotope of iodine in the compound. For example, the term ¹²³I-TAAG-PSMA refers to a compound having the above structure, wherein the I is ¹²³I.

The term I-amino-TAAG-PSMA as used herein refers to a compound having the following structure:

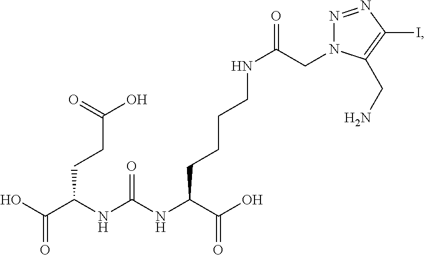 [SMILES: NCc1c(I)nnn1CC(=O)NCCCC[C@H](NC(=O)N[C@@H](CCC(=O)O)C(=O)O)C(=O)O]

wherein a superscript before the I in the term I-amino-TAAG-PSMA indicates the isotope of iodine in the compound. For example, the term ¹²⁵I-amino-TAAG-PSMA refers to a compound having the above structure, wherein the I is ¹²⁵I.

### II. Methods

A prosthetic group referred to herein as the triazole appending agent (TAAG) was studied for use in preparing targeted radioiodine-based molecular imaging and therapy agents. Compounds of Formula II wherein R¹ is H; R² is —CH₂—; L is —C(O)O—; R³ is CH₃; and R⁶, R⁷, R⁸ are all n-Bu or are all (CH₂)₂(CF₂)₅CF₃ were synthesized in high yield using click chemistry and the corresponding acids labeled in greater than 95% RCY with ¹²³I. A compound of Formula II(a) wherein R¹ is H; R² is —CH₂—; L is —C(O)NH—; R⁶, R⁷ and R⁸ are all (CH₂)₂(CF₂)₅CF₃ and A is an inhibitor of PSMA was prepared and radiolabeled with ¹²³I to obtain the corresponding compound of Formula I(a) in 85% yield where biodistribution studies in LNCap prostate cancer tumor models showed rapid clearance of the agent from non-target tissues and tumor accumulation of 20% ID·g⁻¹ at 1 hour. The TAAG group has also been derivatized with targeting vectors comprising tertiary amines capable of binding melanin such as the targeting vector N-(2-diethylaminoethyl)acetamide and a targeting vector that is an N-benzylamino piperadine derivative. Amino-TAAG derivatives have been prepared which provide a second site useful for biomolecule such as the immunogenic moiety 2,4-dinitrophenyl or fluorophore derivatization. TAAG derivatives functionalized with an aryl group have also been prepared. The results of the studies of the present application demonstrate that the TAAG group promotes minimal non-specific binding and that labeled conjugates can achieve high tumor uptake and useful target-to-non-target ratios.

Accordingly, the present application includes a method of preparing a radiohalogenated compound of Formula I:

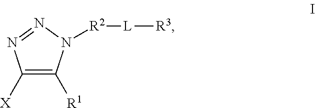

wherein

R¹ is selected from:
(i) H;
(ii) cyano;
(iii) OR⁴;
(iv) NR⁴R⁵;
(v) substituted or unsubstituted C₁₋₆alkyl;
(vi) substituted or unsubstituted C₂₋₆alkenyl;
(vii) substituted or unsubstituted C₃₋₈cycloalkyl;
(viii) substituted or unsubstituted C₃₋₈cycloalkenyl;
(ix) substituted or unsubstituted C₂₋₈heterocycloalkyl;
(x) substituted or unsubstituted C₆₋₁₄aryl; and
(xi) substituted or unsubstituted heteroaryl,
   wherein the substituents for C₁₋₆alkyl, C₂₋₆alkenyl, C₃₋₈cycloalkyl, C₃₋₈cycloalkenyl, C₂₋₈heterocycloalkyl, C₆₋₁₄aryl and heteroaryl are selected from F, Cl, Br, I, cyano, oxo, nitro, OR⁴, NR⁴R⁵, C(O)OR⁴, C(O)N⁴R⁵, C₁₋₄alkyl, C₃₋₈cycloalkyl, C₂₋₈heterocycloalkyl, C₆₋₁₀aryl and an immunogenic moiety;

R² is C₁₋₄alkylene;

R³ is H, C₁₋₄alkyl or a targeting vector;

R⁴ and R⁵ are each independently selected from H, PG, C₁₋₆alkyl, C₂₋₆alkenyl, C₃₋₈cycloalkyl, C₃₋₈cycloalkenyl, C₆₋₁₀aryl, a targeting vector, a fluorophore and an immunogenic moiety; or R⁴ and R⁵ together form PG;

L is an amide linkage or an ester linkage; and

X is a radioisotope of a halogen,

comprising reacting a compound of Formula II:

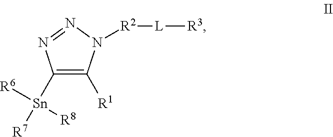

wherein

L, R¹, R² and R³ are as defined for the compound of Formula I; and

R⁶, R⁷ and R⁸ are each independently C₁₋₁₀alkyl or C₁₋₁₀alkyl substituted with one or more F,

with a radiohalogenating agent under conditions to obtain the compound of Formula I.

In embodiments wherein R³ in the compounds of Formula I and II is H or C₁₋₄alkyl, the method can further include reacting the compound of Formula I with a suitable targeting vector under conditions to prepare a radiohalogenated compound of Formula I(a):

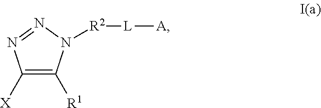

wherein

R¹, R², L and X are as defined for the compound of Formula I; and

A is a targeting vector.

In other embodiments of the present application, R³ in the compounds of Formula I and II is a targeting vector. A person skilled in the art would readily appreciate that such an embodiment is useful because, for example it limits the steps in a method of the present application wherein a radiohalogenated compound is present. This can be useful, for example from a regulatory standpoint. It will also be appreciated by a person skilled in the art that non-radioactive impurities can, for example block a site that is being imaged. Therefore limiting the steps in a method of the present application wherein a radiohalogenated compound is present is also useful, for example as impurities can be removed in steps prior to a step of radiohalogenation. Having the radiohalogenation as a final step in a method of the present application is also useful, for example because such a method can be considered to be a “shake and filter” formulation as opposed to a manufacturing process.

It will be appreciated by a person skilled in the art that certain moieties, for example, a thiol group are sensitive to conditions used for radiohalogenation. Accordingly, in embodiments having a compound comprising a thiol group, the thiol group is in protected form during the step of radiohalogenation and the method comprises a step of removing the protecting group from the thiol group subsequent to the step of radiohalogenation.

The present application also includes a method of preparing a radiohalogenated compound of Formula I(a):

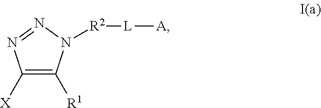

wherein

R¹, R², L and X are as defined for the compound of Formula I; and

A is a targeting vector,

comprising reacting a compound of Formula II(a):

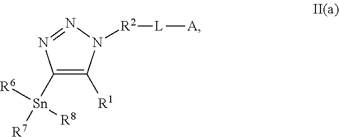

wherein

R¹, R², L and A are as defined for the compound of Formula I(a); and

R⁶, R⁷ and R⁸ are each independently C₁₋₁₀alkyl or C₁₋₁₀alkyl substituted with one or more F,

with a radiohalogenating agent under conditions to obtain the compound of Formula I(a).

The preparation of the compound of Formula II, for example the compound of Formula II(a) can vary and the selection of a suitable method for the preparation of a particular compound of Formula II, for example the compound of Formula II(a) can be made by a person skilled in the art.

For example, the selection of a suitable synthetic route to obtain the ester or amide linkage L in the compound of Formula II, for example the compound of Formula II(a) can be made by a person skilled in the art. A number of synthetic routes are known in the art, for example as described in Smith, M. B. and March J., “March's Advanced Organic Chemistry: Reactions, Mechanisms and Structure” 5th ed., John Wiley & Sons, Inc., 2001 (New York) at, for example, pages 482-486 and pages 506-510.

In an embodiment, L is an ester linkage. For example, a suitable triazole derivative or an azide precursor thereof is reacted with a suitable targeting vector under conditions to obtain an ester linkage. In an embodiment, the conditions to obtain the ester linkage comprise treating a triazole derivative or an azide precursor thereof having a carboxylic acid functional group, optionally under conditions to activate the carboxylic acid, for example by conversion to the acid chloride or by reaction with a carboxylic acid activating reagent, to provide a triazole derivative or an azide precursor thereof having a —C(O)OR^{a} functional group, wherein R^{a} is an activating group for a carboxylic acid, followed by nucleophilic displacement of the —OH, chloride or —OR^{a} group with an oxygen nucleophile (such as a hydroxyl group) on the targeting vector. Carboxylic acid activating reagents are well known in the art and include, for example, well known peptide coupling reagents such as dicyclohexylcarbodiimide (DCC), diisopropylcarbodiimide, hydroxybenzotriazole (HOBT), (Benzotriazol-1-yloxy)tris(dimethylamino)phosphonium hexafluorophosphate (BOP), [N,N,N′,N′-tetramethyl-O-(7-azabenzotriazol-1-yl)uronium hexafluorophosphate (HATU) and the like. A person skilled in the art would appreciate that an ester linkage is also available using the same reaction conditions, but, in the alternative, reacting a triazole derivative or an azide precursor thereof having an oxygen nucleophile such as a hydroxyl group with a targeting vector comprising a carboxylic acid, optionally under conditions to activate the carboxylic acid.

In another embodiment, L is an amide linkage. For example, a suitable triazole derivative or an azide precursor thereof is reacted with a suitable targeting vector under conditions to obtain an amide linkage. In an embodiment, the conditions to obtain the amide linkage comprise treating a triazole derivative or an azide precursor thereof having a carboxylic acid functional group, optionally under conditions to activate the carboxylic acid, for example by conversion to the acid chloride or by reaction with a carboxylic acid activating reagent, to provide a triazole derivative or an azide precursor thereof having a —C(O)OR^{a} functional group, wherein R^{a} is an activating group for a carboxylic acid, followed by nucleophilic displacement of the —OH, chloride or —OR^{a} group with a nitrogen nucleophile (such as an amino group) on the targeting vector. In another embodiment, the conditions to obtain the amide linkage comprise reacting a triazole derivative or an azide precursor thereof having an ester functional group such as —C(O)O—C₁₋₄alkyl, for example —C(O)O—CH₃ with a targeting vector having a nitrogen nucleophile (such as an amino group). A person skilled in the art would appreciate that an amide linkage is also available using the same reaction conditions, but, in the alternative, reacting a triazole derivative or an azide precursor thereof having a nitrogen nucleophile such as an amino group with a targeting vector comprising a carboxylic acid or an ester, optionally under conditions to activate the carboxylic acid.

In an embodiment, L is an ester linkage or an amide linkage obtained by the reaction of a suitable triazole derivative with a suitable targeting vector under conditions to obtain the ester or amide linkage. In another embodiment, L is —C(O)O— or —C(O)NH—. Accordingly, in an embodiment, the method of preparing a radiohalogenated compound of Formula I(a) further comprises preparing the compound of Formula II(a) by steps comprising:

(a) reacting a compound of Formula III:

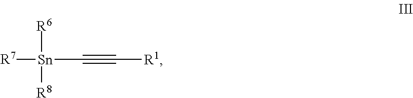

wherein

R¹ is as defined for the compound of Formula I; and

R⁶, R⁷ and R⁸ are each independently C₁₋₁₀alkyl or C₁₋₁₀alkyl substituted with one or more F,

with a compound of Formula IV:

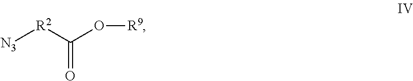

wherein

R² is C₁₋₄alkylene; and

R⁹ is H, C₁₋₄alkyl or an activating group,

under conditions to obtain a compound of Formula V:

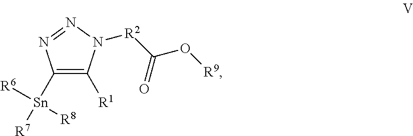

wherein

R¹ is as defined in the compound of Formula I;

R² is C₁₋₄alkylene;

R⁶, R⁷ and R⁸ are each independently C₁₋₁₀alkyl or C₁₋₁₀alkyl substituted with one or more F; and

R⁹ is H, C₁₋₄alkyl or an activating group; and

(b) reacting the compound of Formula V with a compound of Formula VI:

H—R¹⁰-A VI,

wherein

R¹⁰ is O or NH; and

A is a targeting vector,

under conditions to obtain the compound of Formula II(a).

Alternatively, in an embodiment, L is an ester linkage or an amide linkage obtained by the reaction of a suitable azide precursor to a triazole derivative with a suitable targeting vector under conditions to obtain the ester or amide linkage. In another embodiment, L is —C(O)O— or —C(O)NH—. It will be appreciated by a person skilled in the art that the intermediate formed from such a reaction can then be reacted with further reagents under conditions to obtain the desired triazole of Formula II(a). Accordingly, in another embodiment, the method of preparing a radiohalogenated compound of Formula I(a) further comprises preparing the compound of Formula II(a) by steps comprising:

(a) reacting a compound of Formula VI:

H—R¹⁰-A VI,

wherein

R¹⁰ is O or NH; and

A is a targeting vector,

with a compound of Formula IV:

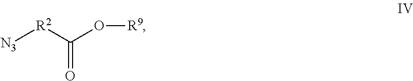

wherein

R² is C₁₋₄alkylene; and

R⁹ is H, C₁₋₄alkyl or an activating group,

under conditions to obtain a compound of Formula VII:

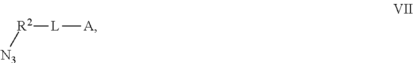

wherein

R² is C₁₋₄alkylene;

L is —C(O)O— or —C(O)NH—; and

A is a targeting vector; and

(b) reacting the compound of Formula VII with a compound of Formula III:

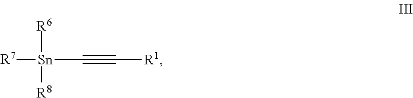

wherein

R¹ is as defined for the compound of Formula I; and

R⁶, R⁷ and R⁸ are each independently C₁₋₁₀alkyl or C₁₋₁₀alkyl substituted with one or more F,

under conditions to obtain the compound of Formula II(a).

In an embodiment, the targeting vector is selected from a substituted amine such as a tertiary amine capable of binding melanin, an enzyme inhibitor such as an inhibitor of prostate specific membrane antigen, a peptide, a protein, an antibody or a fragment thereof, a nucleic acid or an analogue or derivative thereof, a dendrimer and a polymer.

In an embodiment, the targeting vector targets cancer. In another embodiment, the targeting vector is an inhibitor of a protein that is overexpressed in cancer. In another embodiment, the targeting vector binds melanin. In a further embodiment, the cancer is prostate cancer or melanoma. It is an embodiment that the cancer is prostate cancer. In another embodiment of the present application, the cancer is melanoma.

In another embodiment, A is a targeting vector that is in protected form; i.e. it comprises one or more protecting groups. In an embodiment, the one or more protecting groups are t-butyl. It will be appreciated by a person skilled in the art that the one or more protecting groups can be removed at various points in the method. If a step in a method, for example a step which forms the group L and/or which forms a triazole derivative uses conditions under which one or more protecting groups are useful, and a targeting vector that is in protected form is present in the method during such a step, the one or more protecting groups on the targeting vector can be removed, for example, subsequent to such a step. Accordingly, in an embodiment, the one or more protecting groups on the targeting vector are removed subsequent to a step that uses conditions under which one or more protecting groups are useful, for example a step which forms the group L and/or a step which forms a triazole derivative. In another embodiment, the one or more protecting groups on the targeting vector are removed subsequent to the step of radiohalogenation. As detailed above, it is useful to have the step of radiohalogenation as the final step in the method. Accordingly, in an alternative embodiment, the one or more protecting groups on the targeting vector are removed prior to the step of radiohalogenation.

In an embodiment, A is a targeting vector that is in protected form which has the structure:

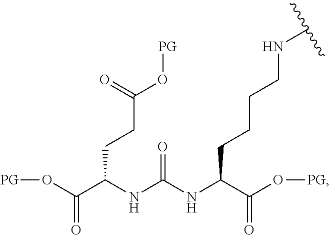

wherein PG is a protecting group for a carboxylic acid. In another embodiment, PG is t-butyl.

In another embodiment, L and A together have the structure:

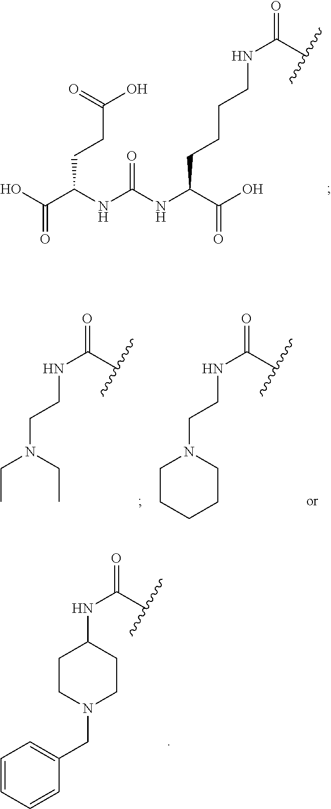 [SMILES: *C(=O)NCCCC[C@H](NC(=O)N[C@@H](CCC(=O)O)C(=O)O)C(=O)O ; *C(=O)NCCN(CC)CC ; *C(=O)NCCN1CCCCC1 ; *C(=O)NC1CCN(Cc2ccccc2)CC1]

It is an embodiment that L and A together have the structure:

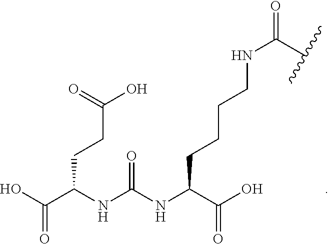 [SMILES: *C(=O)NCCCC[C@H](NC(=O)N[C@@H](CCC(=O)O)C(=O)O)C(=O)O]

In another embodiment, L and A together have the structure:

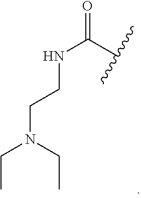 [SMILES: *C(=O)NCCN(CC)CC]

In a further embodiment, L and A together have the structure:

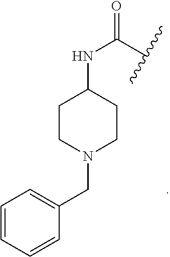 [SMILES: *C(=O)NC1CCN(Cc2ccccc2)CC1]

It is an embodiment that L and A together have the structure:

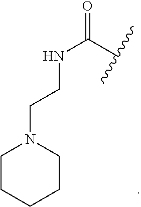 [SMILES: *C(=O)NCCN1CCCCC1]

In an embodiment R¹ is selected from:

(i) H;

(ii) substituted or unsubstituted C₁₋₆alkyl;

(iii) substituted or unsubstituted C₂₋₆alkenyl; and

(iv) substituted or unsubstituted C₆₋₁₄aryl,

wherein the substituents for C₁₋₆alkyl, C₂₋₆alkenyl and C₆₋₁₄aryl are selected from nitro and NR⁴R⁵.

In another embodiment, R¹ is selected from H, substituted C₁₋₄alkyl, C₂₋₆alkenyl and C₆₋₁₀aryl, wherein the substituents for C₁₋₄alkyl are selected from NH₂ and NHR⁴, wherein R⁴ is an immunogenic moiety. In a further embodiment, R¹ is selected from H, substituted C₁₋₄alkyl, C₂₋₆alkenyl and C₆₋₁₀aryl, wherein the substituents for C₁₋₄alkyl are selected from NH₂ and —NH-2,4-dinitrophenyl. In another embodiment, R¹ is selected from H, substituted C₁₋₄alkyl and C₆₋₁₀aryl, wherein the substituents for C₁₋₄alkyl are selected from NH₂ and —NH-2,4-dinitrophenyl. It is an embodiment that R¹ is selected from H, —CH₂NH₂, —CH₂NH-2,4-dinitrophenyl and phenyl. In another embodiment, R¹ is H. In a further embodiment, R¹ is —CH₂NH₂. It is an embodiment that R¹ is —CH₂NH-2,4-dinitrophenyl. In an embodiment, R¹ is phenyl.

In an embodiment, A is a targeting vector that targets cancer, for example, A is an inhibitor of a protein that is overexpressed in cancer and R¹ comprises an immunogenic moiety, for example, R¹ is C₁₋₄alkyl substituted by NHR⁴, wherein R⁴ is an immunogenic moiety. Radiohalogenated compounds comprising a targeting vector that targets cancer and an immunogenic moiety can be useful for both imaging and treatment of the cancer.

In an embodiment, R² is —CH₂—.

In an embodiment, R¹ is H and R² is —CH₂—. In another embodiment, R¹ is —CH₂NH₂ and R² is —CH₂—. In a further embodiment, R¹ is —CH₂NH-2,4-dinitrophenyl and R² is —CH₂—. In another embodiment of the present application, R¹ is phenyl and R² is —CH₂—.

In an embodiment, L is —C(O)O— or —C(O)NH—. In another embodiment, L is —OC(O)— or —NHC(O)—. In a further embodiment, L is —C(O)O— or —OC(O)—. It is an embodiment that L is —C(O)NH— or —NHC(O)—. In an embodiment, L is —C(O)O—. In another embodiment, L is —C(O)NH—. In a further embodiment, L is —OC(O)—. It is an embodiment that L is —NHC(O)—.

In an embodiment, R⁴ and R⁵ together form PG. In an embodiment, PG is phthalimide. It will be appreciated by a person skilled in the art that PG can be removed at various points in the method. As detailed above, it is useful to have the step of radiohalogenation as the final step in the method. Accordingly, in an embodiment, PG is removed prior to the step of radiohalogenation.

In an embodiment, R⁶, R⁷ and R⁸ are each independently C₁₋₆alkyl or C₁₋₈alkyl substituted with one or more F. In another embodiment, R⁶, R⁷ and R⁸ are all n-Bu or are all (CH₂)₂(CF₂)₅CF₃. In a further embodiment R⁶, R⁷ and R⁸ are all n-Bu. It is an embodiment that R⁶, R⁷ and R⁸ are all (CH₂)₂(CF₂)₅CF₃.

In an embodiment, R⁹ is H or C₁₋₄alkyl. In another embodiment, R⁹ is C₁₋₄alkyl. In a further embodiment, R⁹ is CH₃.

In an embodiment, R¹⁰ is O. In another embodiment, R¹⁰ is NH.

In an embodiment, the radiohalogenating agent comprises MX, wherein M is a cation such as an alkali metal cation or an organic cation such as a quaternary amine, for example [NMe₄]⁺ and X is a radioisotope of a halogen. In another embodiment, M is an alkali metal cation. In an embodiment, the radiohalogenating agent comprises NaX or X₂, wherein X is a radioisotope of a halogen. In another embodiment, the radiohalogenating agent comprises NaX, wherein X is a radioisotope of a halogen. In another embodiment, X is selected from a radioisotope of fluorine, bromine, iodine and astatine. In a further embodiment, X is a radioisotope of fluorine, bromine, iodine or astatine used for diagnosis and/or treatment. It is an embodiment that X is selected from ¹⁸F, ¹²³I, ¹²⁴I, ¹²⁵I, ¹³¹I and ²¹¹At. In an embodiment, the radiohalogenating agent is a radioiodinating agent. In an embodiment, the radioiodinating agent comprises I₂ or and iodide (I⁻) salt, such as NaI, wherein I⁻ is a radioisotope of iodine. In another embodiment, the radioiodinating agent comprises NaI, wherein I is a radioisotope of iodine. In an embodiment, the radioisotope of iodine is ¹²³I, ¹²⁴I, ¹²⁵I or ¹³¹I. In another embodiment, the radioisotope of iodine is useful for PET and/or SPECT imaging such as ¹²⁴I or a mixture of ¹³¹I and ¹²³I. It is an embodiment that the radioisotope of iodine is suitable for therapeutic uses such as ¹²⁵I or ¹³¹I. In an embodiment, the radioisotope of iodine is ¹²³I. In another embodiment, the radioisotope of iodine is ¹²⁴I. In a further embodiment, the radioisotope of iodine is ¹²⁵I. It is an embodiment that the radioisotope of iodine is ¹³¹I. In another embodiment, the radioisotope of iodine is a mixture of ¹²³I and ¹³¹I.

In an embodiment, the radioisotope of a halogen is ¹⁸F. It will be appreciated by a person skilled in the art that ¹⁸F is a positron-emissing radioisotope that is useful, for example in positron emission tomography (PET).

In another embodiment, the radioisotope of a halogen is ²¹¹At. It will be appreciated by a person skilled in the art that ²¹¹At decays by the emission of alpha-particles and therefore is useful, for example in targeted radiotherapy.

In an embodiment, the conditions to obtain the compound of Formula I, for example the compound of Formula I(a) comprise adding Na¹²³I to a mixture of the compound of Formula II, for example the compound of Formula II(a) in a suitable solvent such as methanol and a suitable oxidizing agent such as peracetic acid, for example 32 wt % peracetic acid in acetic acid or Iodogen (1,3,4,6-tetrachloro-3α,6α-diphenylglucoluril) and mixing, for example agitating for a time and at a temperature for the conversion of the compound of Formula II to the compound of Formula I to proceed to a sufficient extent, for example about 5 minutes to about 30 minutes, about 5 minutes to about 15 minutes or about 10 minutes at a temperature of about 0° C. to about 60° C., about 10° C. to about 30° C., about 20° C. to about 25° C. or room temperature, followed by a suitable work-up. For example, a mild reductant capable of reducing iodine such as a sulfur-based species, for example sodium metabisulfide or sodium thiosulfate, for example sodium thiosulfate at a concentration of about 0.05 M to about 0.5 M, about 0.05 M to about 0.15 M or about 0.1 M is added and the mixture mixed, for example agitated for a time of about 0.5 minutes to about 5 minutes or about 1 minute at a temperature of about 0° C. to about 60° C., about 10° C. to about 30° C., about 20° C. to about 25° C. or room temperature, followed, for example by concentration to dryness and purification, for example using HPLC.

In an embodiment, the conditions to obtain the compound of Formula V comprise reacting the compound of Formula III with the compound of Formula IV in a suitable solvent, for example a nonpolar solvent such as toluene for a time and at a temperature for the conversion of the compounds of Formula III and IV to the compound of Formula V to proceed to a sufficient extent, for example about 1 hour to about 48 hours, about 1 hour to about 24 hours, about 2 hours to about 10 hours or about 4 hours at refluxing temperatures, for example a temperature of greater than about 100° C., greater than about 100° C. to about 150° C., greater than about 100° C. to about 120° C. or about 111° C. In an embodiment, the conditions to obtain the compound of Formula V comprise reacting the compound of Formula III with the compound of Formula IV in refluxing toluene for a time for the conversion of the compounds of Formula III and IV to the compound of Formula V to proceed to a sufficient extent.

In another embodiment, the conditions to obtain the compound of Formula II(a) comprise reacting the compound of Formula V with a compound of Formula VI in a suitable solvent, for example methanol in the presence of a suitable base such as iPr₂NEt for a time and at a temperature for the conversion of the compounds of Formula V and VI to the compound of Formula II(a) to proceed to a sufficient extent, for example about 1 hour to about 48 hours, about 10 hours to about 36 hours, about 20 hours to about 28 hours or about 24 hours at a temperature of about 10° C. to about 110° C., about 40° C. to about 80° C. or about 60° C.

In an embodiment, the conditions to obtain the compound of Formula VII comprise reacting the compound of Formula VI with the compound of Formula IV in a suitable solvent such as methanol in the presence of a suitable base such as iPr₂NEt for a time and at a temperature for the conversion of the compounds of Formula VI and IV to the compound of Formula VII to proceed to a sufficient extent, for example about 1 hour to about 4 days, about 24 hours to about 3 days, about 36 hours to about 60 hours, or about 48 hours at a temperature of about 10° C. to about 110° C., about 40° C. to about 80° C. or about 60° C.

In another embodiment, the conditions to obtain the compound of Formula II(a) comprise reacting the compound of Formula VII with a compound of Formula III in a suitable solvent such as toluene for a time and at a temperature for the conversion of the compounds of Formula VII and III to the compound of Formula II(a) to proceed to a sufficient extent, for example about 1 hour to about 48 hours, about 4 hours to about 24 hours, about 12 hours to about 20 hours or about 16 hours at a temperature of about 30° C. to about 130° C., about 60° C. to about 100° C. or about 80° C.

Compounds of Formula III may be available from a commercial source or they may be prepared by a suitable method. The selection of a suitable method to prepare a desired compound of Formula III can be made by a person skilled in the art. For example, a compound of Formula III wherein R¹ is H and R⁶, R⁷ and R⁸ are all (CH₂)₂(CF₂)₅CF₃ can be prepared by a method comprising adding I₂ to a solution of (CF₃(CF₂)₅(CH₂)₂)₃SnPh in a suitable solvent such as THF, stirring for a time and at a temperature, for example about 1 minute to about 2 hours, about 5 minutes to about 30 minutes or about 10 minutes at a temperature of about 0° C. to about 60° C., about 10° C. to about 30° C., about 20° C. to about 25° C. or room temperature followed by the addition of ethynylmagnesium bromide, for example dropwise addition over about 30 seconds to about 10 minutes, about 1 minute to about 5 minutes or about 2 minutes at a temperature of about 0° C. to about 60° C., about 10° C. to about 30° C., about 20° C. to about 25° C. or room temperature followed by stirring for a time and at temperature for the conversion of the reactants to the compound of Formula III to proceed to a sufficient extent, for example about 10 minutes to about 24 hours, about 30 minutes to about 4 hours or about 1 hour at a temperature of about 0° C. to about 60° C., about 10° C. to about 30° C., about 20° C. to about 25° C. or room temperature.

For example, in an embodiment, the compound of Formula III wherein R¹ is C₁₋₆alkyl substituted with NR⁴R⁵, wherein R⁴ and R⁵ together form PG, for example wherein R¹ is

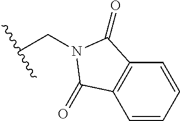 [SMILES: *CN1C(=O)c2ccccc2C1=O]

and R⁶, R⁷ and R⁸ are all n-Bu is prepared by a method comprising the steps of preparing (3-bromoprop-1-yn-1-yl)tributylstannane in accordance with the method reported by Kiyokawa et al.¹³ followed by treatment with potassium phthalimide in accordance with the method reported by Sheehan et al.¹⁴ to give the compound of Formula III. In another embodiment, the compound of Formula III wherein R¹ is

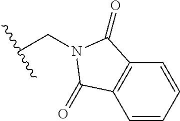 [SMILES: *CN1C(=O)c2ccccc2C1=O]

and R⁶, R⁷ and R⁸ are all n-Bu is prepared by adding Bu₃SnOMe, for example dropwise to a solution of ZnBr₂ and N-propargyl phthalimide in a suitable solvent such as THF followed by stirring for a time and at a temperature for the conversion of the reactants to the compound of Formula III to proceed to a sufficient extent, for example about 30 minutes to about 24 hours, about 1 hour to about 12 hours, about 2 hours to about 4 hours or about 3 hours at a temperature of about 10° C. to about 110° C., about 40° C. to about 80° C. or about 60° C., followed by quenching with a suitable reagent such as water.

In another embodiment, the compound of Formula I is a compound of Formula I(b):

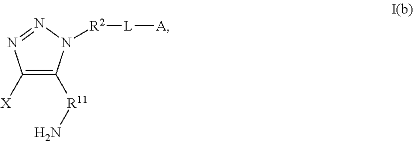

wherein

R² is C₁₋₄alkylene;

L is an amide linkage or an ester linkage;

R¹¹ is C₁₋₄alkylene;

A is a targeting vector; and

X is a radioisotope of a halogen,

and the method further comprises reacting the compound of Formula I(b) with a compound of Formula VIII:

LG-R¹² VIII,

wherein

LG is a leaving group; and

R¹² is an immunogenic moiety,

under conditions to obtain a compound of Formula IX:

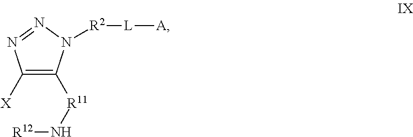

wherein

R² is C₁₋₄alkylene;

L is an amide linkage or an ester linkage;

R¹¹ is C₁₋₄alkylene;

R¹² is an immunogenic moiety;

A is a targeting vector; and

X is a radioisotope of a halogen.

In an embodiment, R¹¹ is —CH₂—.

In an embodiment, LG is halo. In another embodiment, LG is Cl.

In an embodiment, R¹² has the structure:

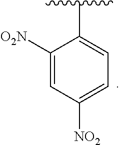 [SMILES: *c1ccc([N+](=O)[O-])cc1[N+](=O)[O-]]

In an embodiment, the compound of Formula VIII has the structure:

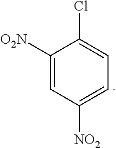 [SMILES: O=[N+]([O-])c1ccc(Cl)c([N+](=O)[O-])c1]

In an embodiment, the conditions to obtain the compound of Formula IX comprise reacting the compound of Formula I(b) and the compound of Formula XIII in a suitable solvent such as ethanol for a time and at a temperature for the conversion of the compounds of Formula I(b) and VIII to the compound of Formula IX to proceed to a sufficient extent, for example about 1 hour to about 48 hours, about 2 hours to about 24 hours, about 4 hours to about 12 hours or about 8 hours at a temperature of about 30° C. to about 130° C., about 60° C. to about 100° C. or about 78° C. In an embodiment, the conditions to obtain the compound of Formula IX comprise reacting the compound of Formula I(b) and the compound of Formula XIII in refluxing ethanol for a time for the conversion of the compounds of Formula I(b) and XIII to the compound of Formula IX to proceed to a sufficient extent.

In another embodiment, the compound of Formula IX is prepared by steps comprising reacting a compound of Formula II(b):

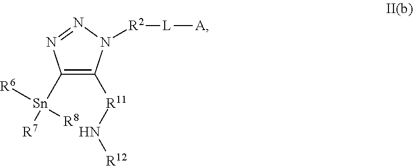

wherein

R² is C₁₋₄alkylene;

L is an amide linkage or an ester linkage;

R⁶, R⁷ and R⁸ are each independently C₁₋₁₀alkyl or C₁₋₁₀alkyl substituted with one or more F;

R¹¹ is C₁₋₄alkylene;

R¹² is an immunogenic moiety; and

A is a targeting vector,

with a radiohalogenating agent under conditions to obtain the compound of Formula IX.

### III. Compounds and Uses

The present application also includes a radiohalogenated compound of Formula I:

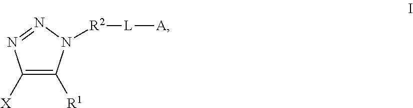

wherein

R¹ is selected from:
(i) H;
(ii) cyano;
(iii) OR⁴;
(iv) NR⁴R⁵;
(v) substituted or unsubstituted C₁₋₆alkyl;
(vi) substituted or unsubstituted C₂₋₆alkenyl;
(vii) substituted or unsubstituted C₃₋₈cycloalkyl;
(viii) substituted or unsubstituted C₃₋₈cycloalkenyl;
(ix) substituted or unsubstituted C₂₋₈heterocycloalkyl;
(x) substituted or unsubstituted C₆₋₁₄aryl; and
(xi) substituted or unsubstituted heteroaryl,
   wherein the substituents for C₁₋₆alkyl, C₂₋₆alkenyl, C₃₋₈cycloalkyl, C₃₋₈cycloalkenyl, C₂₋₈heterocycloalkyl, C₆₋₁₄aryl and heteroaryl are selected from F, Cl, Br, I, cyano, oxo, nitro, OR⁴, NR⁴R⁵, C(O)OR⁴, C(O)N⁴R⁵, C₁₋₄alkyl, C₃₋₈cycloalkyl, C₂₋₈heterocycloalkyl, C₆₋₁₀aryl and an immunogenic moiety;

R² is C₁₋₄alkylene;

R³ is H, C₁₋₄alkyl or a targeting vector;

R⁴ and R⁵ are each independently selected from H, PG, C₁₋₆alkyl, C₂₋₆alkenyl, C₃₋₈cycloalkyl, C₃₋₈cycloalkenyl, C₈₋₁₀aryl, a targeting vector, a fluorophore and an immunogenic moiety; or R⁴ and R⁵ together form PG;

L is an amide linkage or an ester linkage; and

X is a radioisotope of a halogen.

In an embodiment, the compound of Formula I is a compound selected from the following structures:

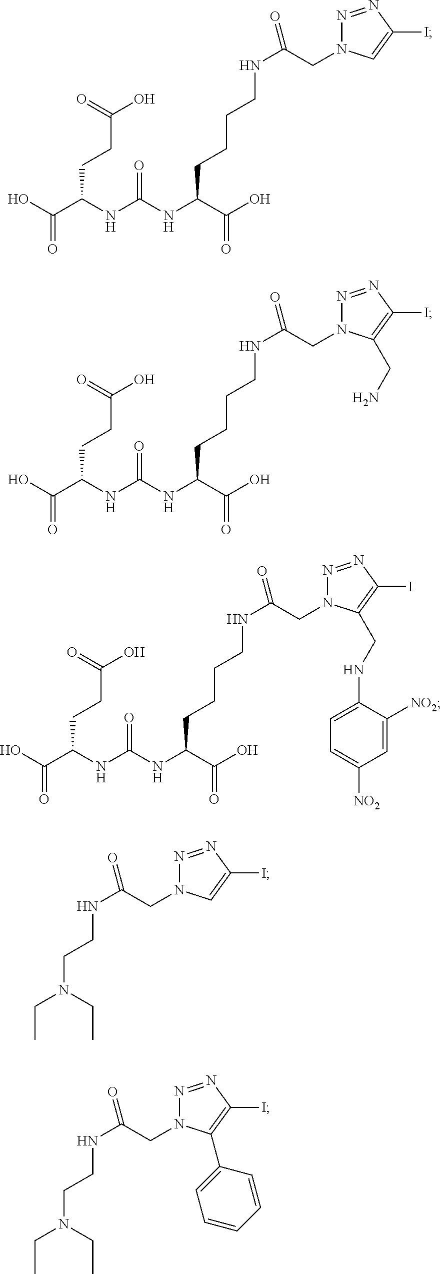

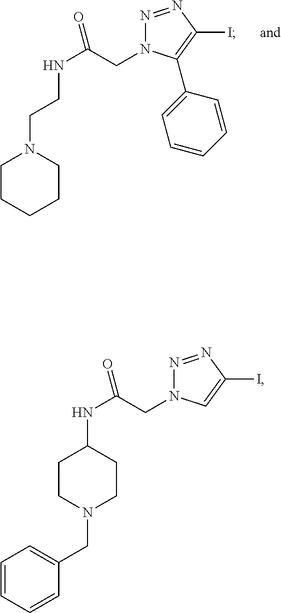

wherein I is a radioisotope of iodine.

In an embodiment, the compound of Formula I is a compound of the following structure:

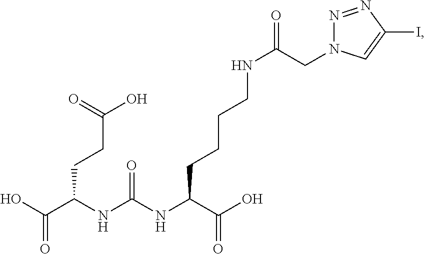

wherein I is a radioisotope of iodine.

In another embodiment, the compound of Formula I is a compound of the following structure:

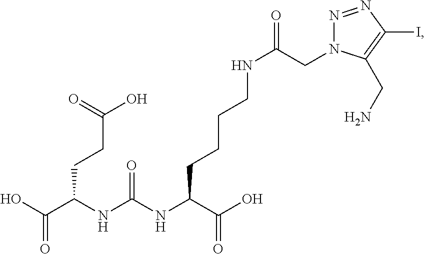

wherein I is a radioisotope of iodine.

In a further embodiment, the compound of Formula I is a compound of the following structure:

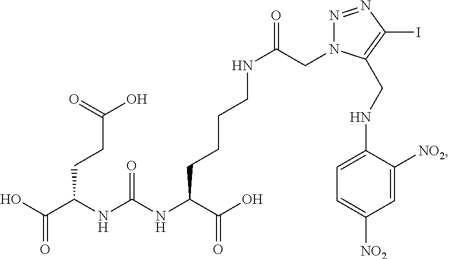

wherein I is a radioisotope of iodine.

It is an embodiment that the compound of Formula I is a compound of the following structure:

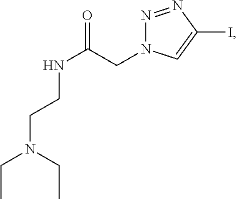

wherein I is a radioisotope of iodine.

In an embodiment, the compound of Formula I is a compound of the following structure:

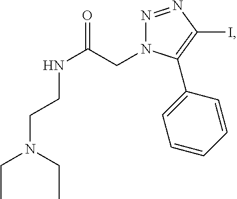

wherein I is a radioisotope of iodine.

In another embodiment, the compound of Formula I is a compound of the following structure:

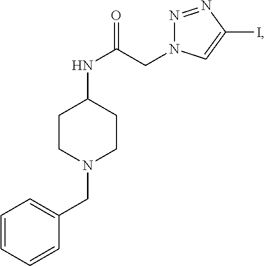

wherein I is a radioisotope of iodine.

In a further embodiment, the compound of Formula I is a compound of the following structure:

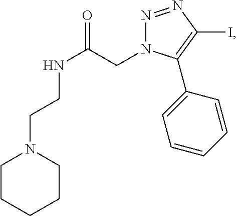

wherein I is a radioisotope of iodine.

The present application also includes a compound of Formula II:

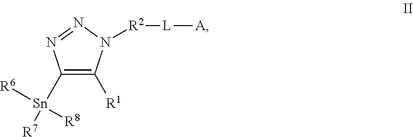

wherein

R¹ is as defined for the compound of Formula I

R² is C₁₋₄alkylene;

L is an amide linkage or an ester linkage;

R⁶, R⁷ and R⁸ are each independently C₁₋₁₀alkyl or C₁₋₁₀alkyl substituted with one or more F; and

R³ is H, C₁₋₄alkyl or a targeting vector.

In an embodiment, the compound of Formula II is a compound selected from the following structures:

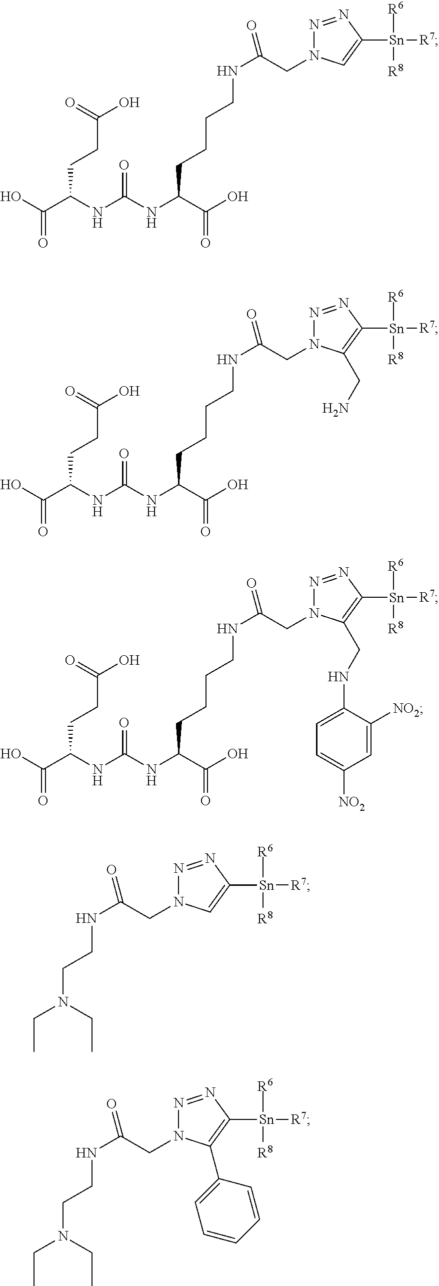

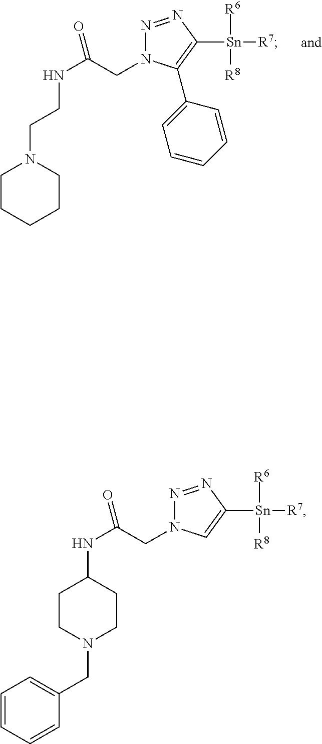

wherein R⁶, R⁷ and R⁸ are all n-Bu or are all (CH₂)₂(CF₂)₅CF₃.

In an embodiment, the compound of Formula II is a compound of the following structure:

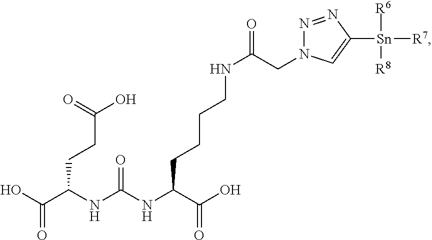

wherein R⁶, R⁷ and R⁸ are all n-Bu or are all (CH₂)₂(CF₂)₅CF₃. In another embodiment, R⁶, R⁷ and R⁸ are all n-Bu. In a further embodiment, R⁶, R⁷ and R⁸ are all (CH₂)₂(CF₂)₅CF₃.

In another embodiment, the compound of Formula II is a compound of the following structure:

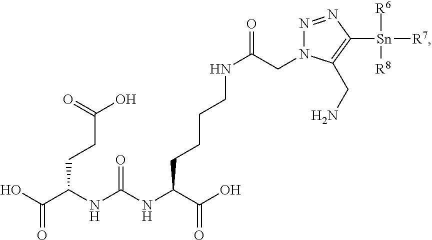

wherein R⁶, R⁷ and R⁸ are all n-Bu or are all (CH₂)₂(CF₂)₅CF₃. In another embodiment, R⁶, R⁷ and R⁸ are all n-Bu. In a further embodiment, R⁶, R⁷ and R⁸ are all (CH₂)₂(CF₂)₅CF₃.

In a further embodiment, the compound of Formula II is a compound of the following structure:

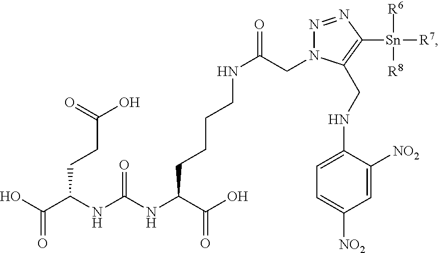

wherein R⁶, R⁷ and R⁸ are all n-Bu or are all (CH₂)₂(CF₂)₅CF₃. In another embodiment, R⁶, R⁷ and R⁸ are all n-Bu. In a further embodiment, R⁶, R⁷ and R⁸ are all (CH₂)₂(CF₂)₅CF₃.

It is an embodiment that the compound of Formula IIIs a compound of the following structure:

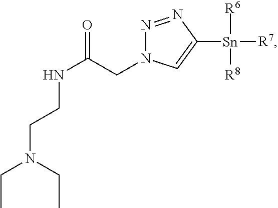

wherein R⁶, R⁷ and R⁸ are all n-Bu or are all (CH₂)₂(CF₂)₅CF₃. In another embodiment, R⁶, R⁷ and R⁸ are all n-Bu. In a further embodiment, R⁶, R⁷ and R⁸ are all (CH₂)₂(CF₂)₅CF₃.

In an embodiment, the compound of Formula II is a compound of the following structure:

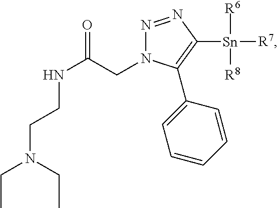

wherein R⁶, R⁷ and R⁸ are all n-Bu or are all (CH₂)₂(CF₂)₅CF₃. In another embodiment, R⁶, R⁷ and R⁸ are all n-Bu. In a further embodiment, R⁶, R⁷ and R⁸ are all (CH₂)₂(CF₂)₅CF₃.

In another embodiment, the compound of Formula II is a compound of the following structure:

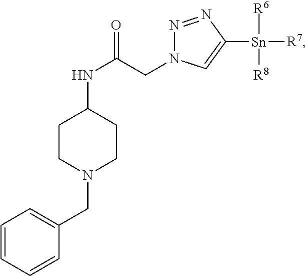

wherein R⁶, R⁷ and R⁸ are all n-Bu or are all (CH₂)₂(CF₂)₅CF₃. In another embodiment, R⁶, R⁷ and R⁸ are all n-Bu. In a further embodiment, R⁶, R⁷ and R⁸ are all (CH₂)₂(CF₂)₅CF₃.

In a further embodiment, the compound of Formula II is a compound of the following structure:

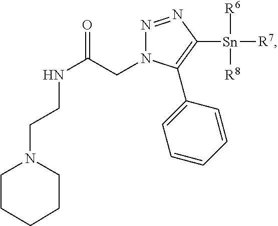

wherein R⁶, R⁷ and R⁸ are all n-Bu or are all (CH₂)₂(CF₂)₅CF₃. In another embodiment, R⁶, R⁷ and R⁸ are all n-Bu. In a further embodiment, R⁶, R⁷ and R⁸ are all (CH₂)₂(CF₂)₅CF₃.

In embodiments of the present application, the variables in the compounds of Formula I and Formula II can also be varied as discussed herein for the methods of the present application.

The compounds of the present application are suitably formulated into compositions. Accordingly, the present application also includes a composition comprising a compound of the present application and a carrier.

The compounds of Formula II of the present application are useful in the preparation of radiohalogenated compounds. Accordingly, the present application also includes a use of a compound of Formula II:

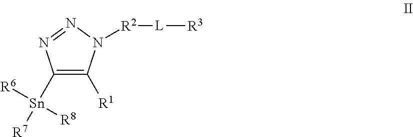

as defined herein for the preparation of a radiohalogenated compound.

The following non-limiting examples are illustrative of the present application:

### EXAMPLES

### Example 1

### Triazole Appending Agents (TAAG)

The feasibility of preparing bifunctional tin-triazoles⁴ that can be linked to targeting vectors such that the bioconjugates can be isolated and ultimately radiolabeled and purified in a single step has been explored herein. The targets included both butyl and fluorous tin derivatives wherein the latter is useful for single step labeling and chemoselective filtration to isolate the desired product in high effective specific activity.⁵

### General Methods

Reactions not involving radioactive material were carried out under argon using flame-dried glassware. ¹H and ¹³C NMR spectra were recorded on a Bruker AV 600 spectrometer. High Resolution Mass Spectra were recorded on a Waters Micromass™ QTOf Global mass spectrometer using electrospray ionization (ESI). IR spectra were obtained on a Biorad FTS-40 FTIR spectrometer. SiliaFlash™ P60 Silica gel from SiliCycle was used for silica gel chromatography. Toluene, dichloromethane and tetrahydrofuran were distilled using a Pure Solv™ distillation system. FC-72 (a mixture of perfluorinated hexanes) was purchased from 3M and (CF₃(CF₂)₅(CH₂)₂)₃SnPh was purchased from Fluorous Technologies Inc. All other reagents were purchased from Sigma-Aldrich and used without further purification. HPLC (analytical and semi-preparative) was performed on a Waters 1525 Binary HPLC system connected to a 2998 photodiode array detector (monitoring at 254 nm) and a Bioscan γ detector. The UV and γ detectors were connected in series. ¹²³I-TAAG was purified and analyzed using an analytical Phenomenex Synergi™ Polar-RP column (150 mm×4.6 mm×4 μm) with a binary solvent gradient (1 mL/min) of 90 to 10% eluent A over 20 min. Eluent A: water containing 0.1% formic acid; Eluent B: ACN containing 0.1% formic acid (method A). ¹²³I-TAAG-PSMA was purified using an Xbridge™ prep C18 column (100 mm×10 mm×5 μm) with a binary solvent gradient (4 mL/min) of 95 to 10% eluent C over 20 min. Eluent C: water containing 0.1% TFA; Eluent D: ACN containing 0.1% triflic acid (method B). ¹²³I-TAAG-PSMA was analyzed using an Xbridge C18 column (100 mm×4.6 mm×3.5 μm) with a binary solvent gradient (1 mL/min) of 95 to 10% eluent C over 20 min. Eluent C: water containing 0.1% TFA; Eluent D: ACN containing 0.1% TFA (method C).

### Synthesis of 1,2,3-Triazole Compounds

I. Exemplary Synthetic Protocols

### a) Preparation of methyl 2-(4-(tributylstannyl)-1H-1,2,3-triazol-1-yl)acetate

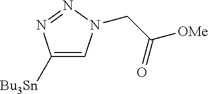

Tributyltinacetylene (2.90 g, 9.20 mmol) was dissolved in toluene (50 mL) followed by the addition of methyl 2-azidoacetate (1.56 g, 13.6 mmol). The reaction mixture was stirred at reflux for 4 h. The reaction was cooled to room temperature, concentrated and purified by silica gel (100 g) chromatography using hexane/ethyl acetate (3:1) as the eluent where the product was isolated as a colorless oil (3.21 g) in 81% yield.

¹H NMR (CDCl₃, 600 MHz) δ 7.57 (s, 1H, SnCCH), 5.18 (s, 2H, NCH₂C), 3.76 (s, 3H, OCH₃), 1.53 (m, 6H, SnCH₂), 1.30 (m, 6H, SnCH₂CH₂), 1.10 (m, 6H, SnCH₂CH₂CH₂), 0.85 (t, 9H, SnCH₂CH₂CH₃); ¹³C NMR (CDCl₃, 150 MHz) δ 167.1, 145.1, 131.2, 52.9, 50.1, 29.0, 27.2, 13.6, 9.9; IR (cm⁻¹) 3120, 2956, 2928, 2872, 2852, 1758; HRMS (ESI⁺) m/z calcd for C₁₇H₃₃N₃O₂Sn: 432.1676 [M+H]⁺, found 432.1666.

### b) Preparation of methyl 2-(4-(tris(1H,1H,2H,2H-perfluorooctyl)stannyl)-1H-1,2,3-triazol-1-yl)acetate

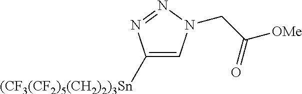

To a solution of (CF₃(CF₂)₅(CH₂)₂)₃SnPh (3.00 g, 2.43 mmol) in THF (20 mL), I₂ (0.735 g, 2.90 mmol) was added as a solid in one portion at room temperature. The reaction mixture was stirred at room temperature for 10 min and 0.5 M ethynylmagnesium bromide in THF (18.0 mL, 9.00 mmol) was then added dropwise at room temperature over 2 min. The reaction was stirred for 1 h at room temperature whereupon the mixture was extracted with FC-72 (3×25 mL). The FC-72 extracts were combined and concentrated by rotary evaporation to yield 2.72 g (94%) of (tris(1H,1H,2H,2H-perfluorooctyl)stannylacetylene) as a colorless viscous oil, which was used without further purification.

To a solution of methyl 2-azidoacetate (0.345 g, 3.00 mmol) in toluene (30 mL) was added (tris(1H,1H,2H,2H-perfluorooctyl)stannylacetylene) (2.40 g, 2.03 mmol) at room temperature and the mixture stirred at reflux for 4 h. Upon completion, the reaction mixture was concentrated by rotary evaporation and purified by silica gel chromatography (˜50 g SiO₂, eluent: hexanes/ethylacetate (2:1)). The fractions containing the desired compound were concentrated to dryness yielding 2.08 g (79%) of the title compound as a colorless oil.

¹H NMR (acetone-d₆, 600 MHz) δ 8.16 (s, 1H, SnCCH), 5.38 (s, 2H, NCH₂C), 3.73 (s, 3H, OCH₃), 2.60 (m, 6H, CF₂CH₂), 1.48 (m, 6H, CH₂Sn); ¹³C NMR (acetone-d₆, 150 MHz) δ 168.4, 142.3, 133.7, 52.9, 50.7, 28.4, 0.0 (C—F signals are not reported); IR (cm⁻¹) 3128, 2961, 2942, 2876, 2110, 1757; HRMS (ESI⁺) m/z calcd for C₂₉H₁₈N₃O₂F₃₉Sn: 1301.9883 [M+H]⁺, found 1301.9902.

### c) Preparation of 2-(4-(tributylstannyl)-1H-1,2,3-triazol-1-yl)acetic acid

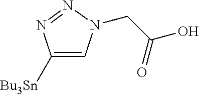

To a solution of methyl 2-(4-(tributylstannyl)-1H-1,2,3-triazol-1-yl)acetate (0.554 g, 1.29 mmol) in 1:1 v/v THF/H₂O (6 mL) was added LiOH (0.154 g, 6.45 mmol). The reaction was stirred at room temperature for 1 h then concentrated to dryness. The organic residue was washed with water (3×30 mL) and the solution decanted. The resulting residue was dried under high vacuum to provide the title compound as a waxy white solid (0.493 g) in 92% yield.

¹H NMR (CD₃OD, 600 MHz) δ 7.88 (s, 1H, SnCCH), 5.01 (s, 2H, NCH₂C), 1.60 (m, 6H, SnCH₂), 1.38 (m, 6H, SnCH₂CH₂), 1.17 (m, 6H, SnCH₂CH₂CH₂), 0.78 (t, 9H, SnCH₂CH₂CH₂CH₃); ¹³C NMR (CD₃OD, 150 MHz) δ 173.1, 144.6, 133.6, 54.0, 30.2, 28.3, 14.0, 10.7; IR (cm⁻¹) 3409 (br), 3112, 2956, 2926, 2871, 2853, 1634, 1401; HRMS (ESI⁺) m/z calcd for C₁₆H₃₁N₃O₂Sn: 418.1519 [M+H]⁺, found 418.1500.

### d) Preparation of 2-(4-(tris(1H,1H,2H,2H-perfluorooctyl)stannyl)-1H-1,2,3-triazol-1-yl)acetic acid

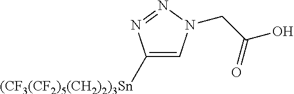

To a solution of methyl 2-(4-(tris(1H,1H,2H,2H-perfluorooctyl)stannyl)-1H-1,2,3-triazol-1-yl)acetate (0.393 g, 0.302 mmol) in 1:1 v/v THF/H₂O solution (6 mL) was added LiOH (0.036 g, 1.503 mmol). The reaction mixture was stirred at room temperature for 30 min. Following concentration by rotary evaporation, the residue, a white solid, was washed with water (3×25 mL) and decanted. The resulting solid was dried under high vacuum to provide the title compound as a white film (0.345 g) in 89% yield.

¹H NMR (CD₃OD, 600 MHz) δ 7.96 (s, 1H, SnCCH), 5.02 (s, 2H, NCH₂C), 2.46 (m, 6H, CF₂CH₂), 1.38 (m, 6H, CH₂Sn); ¹³C NMR (CD₃OD, 150 MHz) δ 172.8, 142.5, 134.0, 53.9, 28.5, 0.00; IR (cm⁻¹) 2945, 1677, 1610; HRMS (ESI⁺) m/z calcd for C₂₈H₁₆N₃O₂F₃₉Sn: 1287.9725 [M+H]⁺, found 1287.9755.

### e) Preparation of 2-(4-iodo-1H-1,2,3-triazol-1-yl)acetic acid

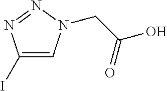

To a solution of 2-(4-(tributylstannyl)-1H-1,2,3-triazol-1-yl)acetic acid (0.224 g, 0.52 mmol) in THF (3 mL), I₂ (0.053 g, 0.21 mmol) was added. The reaction mixture was stirred at room temperature for 10 min, which was followed by evaporation of the solvent to give a white solid which was redissolved in H₂O/MeOH (1:1 v/v) and purified by flash chromatography (˜15 g of SiO₂, eluent: DCM/MeOH (1:1 v/v)). The title compound was isolated as a white solid (0.125 g, 95%).

¹H NMR (D₂O, 600 MHz) δ 7.98 (s, 1H, ICCH), 4.97 (s, 2H, NCH₂C); ¹³C NMR (D₂O, 150 MHz) δ 172.8, 132.2, 86.7, 53.2; IR (cm⁻¹) 3148, 3128, 1632, 1396; HRMS (ES⁺) m/z calcd for C₄H₄N₃O₂I: 253.9427 [M+H]⁺, found 253.9432.

### f) Preparation of (S)-di-tert-butyl 2-(3-((S)-1-tert-butoxy-1-oxo-6-(2-(4-(tributylstannyl)-1H-1,2,3-triazol-1-yl)acetamido)hexan-2-yl)ureido)pentanedioate

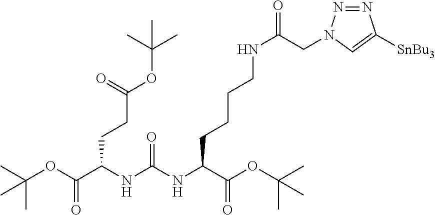

To a solution of methyl 2-(4-(tributylstannyl)-1H-1,2,3-triazol-1-yl)acetate (0.176 g, 0.409 mmol) in MeOH (4 mL) was added t-butyl-protected glutamate-urea-lysine (0.257 g, 0.527 mmol) followed by iPr₂NEt (0.21 mL, 1.21 mmol). The reaction was stirred at 60° C. for 24 h. The reaction mixture was evaporated to dryness and purified by silica gel chromatography using hexane/ethyl acetate as eluents (1:2 v/v) to provide the title compound as a colorless oil (0.264 g) in 73% yield.

¹H NMR (CDCl₃, 600 MHz) δ 7.64 (s, 1H), 6.86 (m, 1H), 5.62 (d, J=6.0 Hz, 1H), 5.31 (d, J=6.0 Hz, 1H), 5.15 (d, J=12 Hz, 1H), 5.04 (d, J=12 Hz, 1H), 4.31 (m, 1H), 4.24 (m, 1H), 3.33 (m, 1H), 3.10 (m, 1H), 2.31 (m, 2H), 2.07 (m, 1H), 1.82 (m, 3H), 1.71 (m, 1H), 1.48 (m, 7H), 1.43 (m, 27H), 1.32 (m, 8H), 1.11 (m, 6H), 0.85 (t, 9H); ¹³C NMR (CDCl₃, 150 MHz) δ 173.1, 172.4, 172.3, 165.9, 157.3, 145.3, 131.6, 82.1, 81.6, 80.6, 53.2, 53.1, 52.5, 38.9, 31.9, 31.7, 28.9, 28.2, 28.08, 28.07, 28.03, 28.02, 27.2, 21.9, 13.7, 10.0; IR (cm⁻¹) 3325, 3092, 2957, 2930, 2873, 2854, 1731, 1658, 1554; HRMS (ESI⁺) m/z calcd for C₄₀H₇₄N₆O₈Sn: 909.4497 [M+Na]⁺, found 909.4482.

### g) Preparation of (S)-di-tert-butyl 2-(3-((S)-1-tert-butoxy-1-oxo-6-(2-(4-(tris(1H,1H,2H,2Hperfluorooctyl)stannyl)-1H-1,2,3-triazol-1-yl)acetamido)hexan-2-yl)ureido)pentanedioate

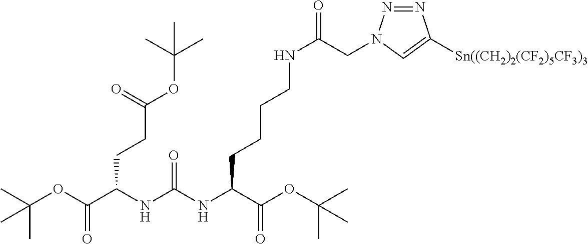

To a solution of methyl 2-(4-(tris(1H,1H,2H,2H-perfluorooctyl)stannyl)-1H-1,2,3-triazol-1-yl)acetate (0.250 g, 0.192 mmol) in MeOH (4 mL) was added t-butyl-protected glutamate-urea-lysine (0.121 g, 0.249 mmol) followed by iPr₂NEt (0.100 mL, 0.576 mmol). The reaction was stirred at 60° C. for 24 h. The reaction mixture was evaporated to dryness and purified by silica gel chromatography using hexane/ethyl acetate as eluents (1:1 v/v) to provide the title compound as a colorless oil (0.261 g) in 77% yield.

¹H NMR (Acetone-d₆, 600 MHz) δ 8.13 (s, 1H), 7.54 (s, 1H), 5.94 (d, J=12 Hz, 1H), 5.91 (d, J=6.0 Hz, 1H), 5.17 (s, 2H), 4.28 (dt, J=8.5 Hz, J=5.0 Hz, 1H), 4.20 (dt, J=8.2 Hz, J=5.0 Hz, 1H), 3.22 (dd, J=12.8 Hz, J=6.8 Hz, 2H), 2.62 (m, 6H), 2.29 (m, 2H), 2.07 (m, 1H), 1.80 (m, 1H), 1.73 (m, 1H), 1.57 (m, 4H), 1.49-1.41 (m, 34H); ¹³C NMR (Acetone-d₆, 150 MHz) δ 173.2, 173.1, 172.5, 166.2, 156.2, 142.0, 133.7, 81.7, 81.4, 80.5, 54.2, 53.8, 52.5, 39.9, 33.3, 32.1, 29.1, 28.4, 28.32, 28.27, 28.25, 23.5, 0.0; IR (cm⁻¹) 3325 (br), 3103, 2981, 2934, 2870, 1731, 1652, 1558; HRMS (ESI⁺) m/z calcd for C₅₂H₅₉F₃₉N₆O₈Sn: 1757.2885 [M+H]⁺, found 1757.2894.

### h) Preparation of (S)-di-tert-butyl 2-(3-((S)-1-tert-butoxy-6-(2-(4-iodo-1H-1,2,3-triazol-1-yl)acetamido)-1-oxohexan-2-yl)ureido)pentanedioate

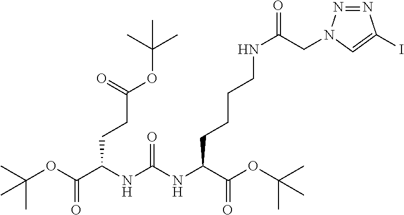 [SMILES: CC(C)(C)OC(=O)CC[C@H](NC(=O)N[C@@H](CCCCNC(=O)Cn1cc(I)nn1)C(=O)OC(C)(C)C)C(=O)OC(C)(C)C]

To a solution of (S)-di-tert-butyl 2-(3-((S)-1-tert-butoxy-1-oxo-6-(2-(4-(tributylstannyl)-1H-1,2,3-triazol-1-yl)acetamido)hexan-2-yl)ureido)pentanedioate (0.044 g, 0.050 mmol) in DCM (3 mL) was added I₂ (0.013 g, 0.052 mmol) at room temperature and the mixture was stirred for 15 min. The reaction was passed through a silica plug (2 g, 10 wt % KF mixed in silica) and concentrated under vacuum to dryness and purified by silica gel chromatography using hexane/ethyl acetate as eluents (1:2) to provide the title compound as a colorless oil (0.029 g) in 81% yield.

¹H NMR (Acetone-d₆, 600 MHz) δ 8.17 (s, 1H), 5.91 (dd, J=14.7 Hz, J=8.3 Hz, 2H), 5.19 (s, 2H), 4.26 (dt, J=8.5 Hz, J=5.1 Hz, 1H), 4.18 (dt, J=8.2 Hz, J=5.1 Hz, 1H), 3.22 (dt, J=6.9 Hz, J=2.3 Hz, 2H), 2.29 (m, 2H), 2.01 (m, 1H), 1.76 (m, 2H), 1.56 (m, 4H), 1.44-1.41 (m, 29H); ¹³C NMR (Acetone-d₆, 150 MHz) δ 173.1, 172.9, 172.4, 165.7, 158.1, 132.3, 87.1, 81.6, 81.3, 80.4, 54.1, 53.7, 53.0, 38.9, 33.2, 32.0, 29.0, 28.26 (tBu 3C), 28.24, 28.20 (tBu, 6C), 23.3; IR (cm⁻¹) 3333 (br), 3134, 3092, 2977, 2933, 2867, 1729, 1653, 1557, 1478, 1455; HRMS (ESI⁺) m/z calcd for C₂₈H₄₇IN₆O₈: 723.2578 [M+H]⁺, found 723.2544.

### i) Preparation of (S)-2-(3-((S)-1-carboxy-5-(2-(4-iodo-1H-1,2,3-triazol-1-yl)acetamido)pentyl)ureido)pentanedioic acid

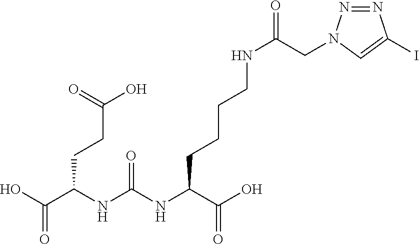 [SMILES: O=C(O)CC[C@H](NC(=O)N[C@@H](CCCCNC(=O)Cn1cc(I)nn1)C(=O)O)C(=O)O]

To a solution of (S)-di-tert-butyl 2-(3-((S)-1-tert-butoxy-1-oxo-6-(2-(4-(tributylstannyl)-1H-1,2,3-triazol-1-yl)acetamido)hexan-2-yl)ureido)pentanedioate (0.056 g, 0.064 mmol) in DCM (3 mL) was added I₂ (0.018 g, 0.071 mmol) at room temperature and the mixture was stirred for 15 min. The reaction was passed through a silica plug (2 g, 10 wt % KF mixed in silica) and concentrated under vacuum. The organic residue was redissolved in DCM (3 mL) and treated with TFA (3 mL) and stirred at room temperature for 18 h. The reaction mixture was concentrated and was purified by semi-preparative HPLC (method B) followed by evaporation. Purified material was dissolved in water and lyophilised to provide the title compound as a white paste (0.025 g) in 71% yield.

¹H NMR (D₂O, 600 MHz) δ 8.03 (s, 1H), 5.14 (s, 2H), 4.13 (dd, J=9.1 Hz, J=5.0 Hz, 1H), 4.06 (dd, J=8.9, J=4.9 Hz, 1H), 3.13 (t, J=6.8 Hz, 2H), 2.39 (t, J=7.3 Hz, 2H), 2.05 (m, 1H), 1.86 (m, 1H), 1.71 (m, 1H), 1.59 (m, 1H), 1.43 (m, 2H), 1.27 (m, 2H); ¹³C NMR (D₂O, 150 MHz) δ 177.3, 177.2, 176.4, 167.3, 159.3, 132.6, 87.0, 53.2, 52.7, 52.2, 39.3, 30.5, 30.1, 27.6, 26.3, 22.2; IR (cm⁻¹) 3304, 3091, 2939, 2865, 1723, 1632, 1564; HRMS (ESI⁺) m/z calcd for C₁₆H₂₃IN₆O₈: 555.0701 [M+H]⁺, found 555.0688.

II. Radiolabeling

### a) Radiolabeling 2-(4-(tris(1H,1H,2H,2H-perfluorooctyl)stannyl)-1H-1,2,3-triazol-1-yl)acetic acid with Na¹²³I

2-(4-(tris(1H,1H,2H,2H-perfluorooctyl)stannyl)-1H-1,2,3-triazol-1-yl)acetic acid (100 μL, 5 mg/mL in MeOH) and 5 μL of peracetic acid (32 wt % in AcOH) were added to a 1.5 mL eppendorf tube. To the reaction mixture was added [Na¹²³I] (10 μL, 2 mCi) and the mixture was shaken periodically for 10 min. To this was added 0.1 M sodium thiosulfate (100 μL) and the mixture was agitated again for 1 min followed by concentration to dryness using a Biotage V10 evaporator. The desired product was purified by HPLC using method A. ¹²³I-TAAG was obtained in 95% radiochemical yield and >99% radiochemical purity.

### b) Radiolabeling (S)-di-tert-butyl 2-(3-((S)-1-tert-butoxy-1-oxo-6-(2-(4-(tris(1H,1H,2H,2Hperfluorooctyl)stannyl)-1H-1,2,3-triazol-1-yl)acetamido)hexan-2-yl)ureido)pentanedioate with Na¹²³I

(S)-d i-tert-butyl 2-(3-((S)-1-tert-butoxy-1-oxo-6-(2-(4-(tris(1H,1H,2H,2Hperfluorooctyl)stannyl)-1H-1,2,3-triazol-1-yl)acetamido)hexan-2-yl)ureido)pentanedioate (100 μL, 5 mg/mL in MeOH) and 5 μL of peracetic acid (32 wt % in AcOH) were added to a 1.5 mL eppendorf tube. To the reaction mixture was added [Na¹²³I] (10 μL, 2 mCi) and the mixture was shaken for 10 min. To this was added 0.1 M sodium thiosulfate (100 μL) and the mixture was agitated for 1 min. The reaction mixture was transferred to a scintillation vial (20 mL) and concentrated using a V10 Biotage evaporator. To the vial were added 400 μL of ACN and 2 mL of TFA, followed by stirring for 1 h at room temperature. The reaction mixture was concentrated using a Biotage V10 evaporator, diluted with 500 μL of H₂O and purified by HPLC using method B. ¹²³I-TAAG-PSMA was obtained in 85% radiochemical yield and >99% radiochemical purity.

III. Formulation for Biodistribution Studies

### a) Formulation for Biodistribution Study of ¹²³I-TAAG

The HPLC fraction containing ¹²³I-TAAG (˜1 mL) was concentrated using a Biotage V10 evaporator, diluted with 1 mL of deionized H₂O and then loaded onto an anion-exchange Sep-Pak® (Waters Accell™ Plus QMA Cartridge). The cartridge was washed with 5 mL of H₂O then ¹²³I-TAAG was eluted using PBS (1.06 mM K phosphate monobasic, 155.17 mM NaCl, 2.97 mM Na phosphate dibasic). The formulation was analyzed for purity by HPLC using method A.

### b) Formulation for Biodistribution and Imaging Studies of ¹²³I-TAAG-PSMA

The HPLC fraction containing ¹²³I-TAAG-PSMA (˜1 mL) was concentrated using a Biotage V10 evaporator, diluted with 1 mL of deionized H₂O and then loaded onto an anion-exchange SepPak® (Waters Accell™ Plus QMA Cartridge). The cartridge was washed with 5 mL of H₂O then ¹²³I-TAAG-PSMA was eluted using PBS (1.06 mM K phosphate monobasic, 155.17 mM NaCl, 2.97 mM Na phosphate dibasic). The formulation was analyzed for purity by HPLC using method C.

IV. Log D_{pH 7.4} Measurement

PBS (10 μL) containing ¹²³I-TAAG or ¹²³I-TAAG-PSMA (5 μCi) was added to an equal volume mixture of n-octanol and PBS (pH 7.4). The samples were vortexed for 20 min and centrifuged at 6000 rpm for 5 min. Aliquots (0.1 mL) were subsequently removed from both the aqueous and the organic n-octanol layers and counted separately in a γ counter (Wallac Wizard 1470 automatic γ counter). Measurements represent three separate experiments with four extractions in each set for a total of twelve data points. The partition coefficients were then calculated using the equation D=(activity concentration in n-octanol)/(activity concentration in aqueous layer). The log D_{pH 7.4} values were determined to be −2.58±0.01 for ¹²³I-TAAG and −3.23±0.05 for ¹²³I-TAAG-PSMA.

V. Biodistribution Studies

### a) Biodistribution Study of ¹²³I-TAAG

Biodistribution of ¹²³I-TAAG was performed using 7-8 week old female Balb/c mice ordered from Charles River Laboratories (Senneville, QC, Canada), (n=5 per time point at t=0.5, 1.0, 2, 18 h). The mice were administered ˜1.30 MBq of ¹²³I-TAAG (100 μL in PBS) via tail vein injection. Animals were anesthetized with 3% isoflurane and euthanized by cervical dislocation. Blood, heart, lungs, liver, spleen, kidneys, adrenals, stomach (with contents), large intestine and caecum (with contents) and small intestines (with contents), bladder and urine, thyroid/trachea, bone, skeletal muscle and tail were collected, weighed and counted in a Perkin Elmer Wizard 1470 Automatic Gamma Counter. Decay correction was used to normalize organ activity measurements to time of dose preparation for data calculations with respect to injected dose (i.e. % ID/g).

### b) Biodistribution Study of ¹²³I-TAAG-PSMA

LNCaP cells derived from lymph node metastases of human prostate carcinoma were purchased from ATCC. Cells were propagated using RPMI 1640 media supplemented with 10% Fetal Bovine Serum, 4 mM L-glutamine, 1 mM Sodium Pyruvate, 10 mM Hepes, 1% Penicillin Streptomycin (Invitrogen, Mississauga ON) and 0.25% D-glucose (Sigma, Oakville ON) and grown at 37° C. and 5% CO₂. NCr nude homo male mice ordered from Taconic (Germantown, N.Y., USA) were injected with 2.0×10⁶ LNCaP cells in 100 μL Matrigel/DPBS (1:1) subcutaneously into the right flank at 7 to 8 weeks of age. Biodistribution of ¹²³I-TAAG-PSMA was performed on mice at 5 weeks post inoculation (n=5 for t=1 h, n=3 for t=1 h+block and n=4 for t=24 h). Two groups (t=1 h and 1=24 h) were injected with 50 μL of saline immediately followed with an second injection containing approximately 0.63 MBq of ¹²³I-TAAG-PSMA in PBS via the tail vein. A third cohort received 50 μL of 5 mg/mL PMPA (a specific PSMA inhibitor; Cedarlane Laboratories, Burlington, ON) in saline (equivalent to a 10 mg/kg dose) immediately followed by a second injection containing approximately 0.63 MBq of ¹²³I-TAAG-PSMA (t=1 h+block). Animals were anesthetized with 3% isoflurane and euthanized by cervical dislocation. Blood, bone, liver, gall bladder, spleen, small intestine (with contents), large intestine and caecum (with contents), bladder, urine, skeletal muscle, brain, LNCaP tumour, heart, lung, salivary glands, kidneys, stomach (with contents), prostate, thyroid/trachea, adrenals, esophagus, tail, adipose and testes were collected, weighed and counted in a Perkin Elmer Wizard 1470 Automatic Gamma Counter. Decay correction was used to normalize organ activity measurements to time of dose preparation for data calculations with respect to injected dose (i.e. % ID/g).

The tissue distribution of ¹²³I-TAAG-PSMA in NCr nude mice bearing LNCaP xenografts is shown in Table 1. Tumor to tissue ratios for ¹²³I-TAAG-PSMA in NCr nude mice bearing LNCaP xenografts are shown in Table 2.

VI. Imaging Study

Imaging of ¹²³I-TAAG-PSMA was completed using two 13-14 week old male NCr mice bearing 6 week old LNCaP tumours. Both mice were administered 200 μl of PBS containing ¹²³I-TAAG-PSMA (˜37.0 MBq) via tail vein injection. Prior to imaging, mice were anaesthetized with 2.5% isoflurane and maintained under same conditions for the length of the SPECT and CT scans. Imaging was conducted on the first mouse at 2 and 6 hours post injection and on the second mouse at 4 and 23 hours post injection. At each time point, multiple SPECT acquisitions were completed for 32 frames at 10 sec/frame for the 2 hour time point, 20 sec/frame for the 4 and 6 hour time point or 30 sec/frame for the 23 hour time point on a GammaMedica Ideas X-SPECT system (North Ridge, Calif.). CT acquisitions consisted of 512 projections acquired over 360° with 75 Kvp, 205 mA cone beam CT system. Cobra Exxim software (Feldkamp filtered backprojection cone beam reconstruction software) was used to reconstruct the images at a voxel size of 155 microns and a matrix size of 512³. An OS-EM interactive reconstructed method (2 iterations/8 subsets) was used to reconstruct the SPECT data which was fused to the CT data using in-house software. AMIDE software was used to analyze the images.

### III. Discussion

A versatile and stable class of compounds referred to herein as the triazole appending agents (TAAG) that can be used to develop probes that clear rapidly from non-target tissues and/or that are able to penetrate tumour cells when appended to an appropriate vector are reported.

Tributyltinacetylene is commercially available and readily undergoes a catalyst-free cycloaddition reaction with methyl 2-azidoacetate. Heating tributyltinacetylene and the azide methyl 2-azidoacetate in toluene at reflux afforded the ester methyl 2-(4-(tributylstannyl)-1H-1,2,3-triazol-1-yl)acetate in 81% yield where, following chromatographic purification only a single isomer was isolated. The product is stable for several months when stored in the freezer and protected from light. The fluorous acetylene analogue was prepared by treating commercially available tris(1H,1H,2H,2H-perfluorooctyl)phenyltin with iodine followed by ethynyl magnesium bromide. The product (tris(1H,1H,2H,2H-perfluorooctyl)stannylacetylene) can be isolated or immediately combined with the appropriate azide where in the case of methyl 2-azidoacetate, methyl 2-(4-(tris(1H,1H,2H,2H-perfluorooctyl)stannyl)-1H-1,2,3-triazol-1-yl)acetate was obtained in 79% yield. There was no notable reactivity difference between the fluorous and alkyl tin derivatives.

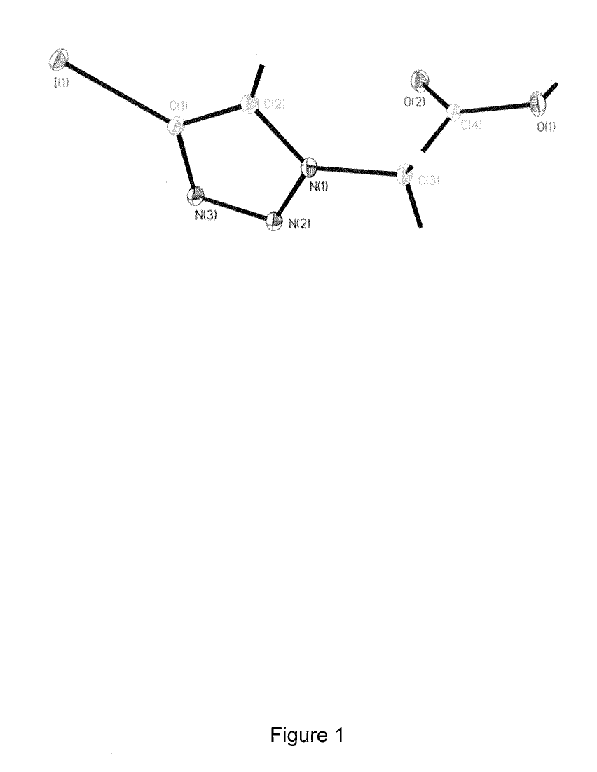

The methyl esters methyl 2-(4-(tributylstannyl)-1H-1,2,3-triazol-1-yl)acetate and methyl 2-(4-(tris(1H,1H,2H,2H-perfluorooctyl)stannyl)-1H-1,2,3-triazol-1-yl)acetate were hydrolysed using aqueous LiOH in greater than 90% yield to give the corresponding acids 2-(4-(tributylstannyl)-1H-1,2,3-triazol-1-yl)acetic acid and 2-(4-(tris(1H,1H,2H,2H-perfluorooctyl)stannyl)-1H-1,2,3-triazol-1-yl)acetic acid, respectively. Iodination of 2-(4-(tributylstannyl)-1H-1,2,3-triazol-1-yl)acetic acid produced the reference standard (2-(4-iodo-1H-1,2,3-triazol-1-yl)acetic acid) used for the radiochemical reactions in 95% yield. X-ray quality crystals were obtained and the structure (FIG. 1), which was consistent with the ¹H and ¹³C NMR data, showed that a single isomer was isolated. The length of the C—I bond in the triazole was 2.0617(18) Å compared to 2.097(9) Å in p-iodobenzoic acid.⁶ All bond lengths and angles were comparable to other reported triazoles.⁷ Table 3 summarizes the crystal data and structure refinement, Table 4 provides details on the bond lengths and angles, and Table 5 provides details on the torsion angles for ¹²⁷I-TAAG (2-(4-iodo-1H-1,2,3-triazol-1-yl)acetic acid).

To test the reactivity and stability of the I-TAAG construct, 2-(4-(tributylstannyl)-1H-1,2,3-triazol-1-yl)acetic acid was treated with Na¹²³I in the presence of peracetic acid. Using 500 μg of precursor, the desired product ¹²³I-TAAG was obtained in greater than 95% radiochemical yield and greater than 99% radiochemical purity. Reactions were complete within 10 min and the product was isolated by solid-phase extraction or semi-preparative HPLC.

The log D of ¹²³I-TAAG was determined to be −2.58±0.01 (at pH 7.4) which is more hydrophilic than p-iodobenzoic acid (log D of 0.07 at pH 7.4). ¹²³I-TAAG was stable over 48 hours in solution with no signs of deiodination.

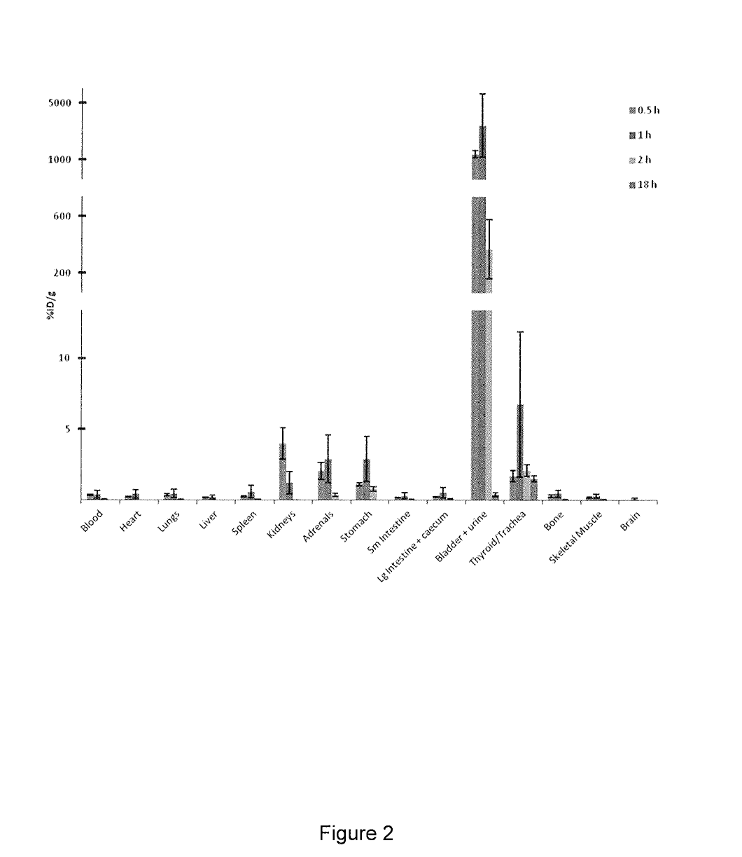

A biodistribution study was performed to assess the extent to which the agent de-iodinates in vivo and binds non-specifically to key tissues such as the kidneys and liver. ¹²³I-TAAG clears all major organs quickly and collects in the bladder within 30 minutes (FIG. 2). This is a useful feature of a prosthetic group being used to develop targeted imaging and therapy agents.⁸

As a model targeting vector, a glutamate-urea-lysine analogue was chosen which is an inhibitor of prostate-specific membrane antigen (PSMA); a protein that is overexpressed in prostate cancer.⁹ The glu-urea-lys construct has been derivatized with different radioisotopes including iodoaryl compounds and was therefore a useful agent for assessing the iodotriazole synthon.¹⁰

Methyl ester methyl 2-(4-(tributylstannyl)-1H-1,2,3-triazol-1-yl)acetate was coupled to tBu protected glu-urea-lys at 60° C. for 24 hours and the product (S)-d i-tert-butyl 2-(3-((S)-1-tert-butoxy-1-oxo-6-(2-(4-(tributylstannyl)-1H-1,2,3-triazol-1-yl)acetamido)hexan-2-yl)ureido)pentanedioate isolated in 73% yield. The fluorous analogue (S)-di-tert-butyl 2-(3-((S)-1-tert-butoxy-1-oxo-6-(2-(4-(tris(1H,1H,2H,2Hperfluorooctyl)stannyl)-1H-1,2,3-triazol-1-yl)acetamido)hexan-2-yl)ureido)pentanedioate was prepared using a similar method from the ester methyl 2-(4-(tris(1H,1H,2H,2H-perfluorooctyl)stannyl)-1H-1,2,3-triazol-1-yl)acetate in 77% yield. The iodine standard (S)-2-(3-((S)-1-carboxy-5-(2-(4-iodo-1H-1,2,3-triazol-1-yl)acetamido)pentyl)ureido)pentanedioic acid was prepared by treating (S)-di-tert-butyl 2-(3-((S)-1-tert-butoxy-1-oxo-6-(2-(4-(tributylstannyl)-1H-1,2,3-triazol-1-yl)acetamido)hexan-2-yl)ureido)pentanedioate with I₂ followed by deprotection using TFA where the product was isolated in 71% yield.

The PSMA-TAAG-tin ligand (S)-di-tert-butyl 2-(3-((S)-1-tert-butoxy-1-oxo-6-(2-(4-(tris(1H,1H,2H,2Hperfluorooctyl)stannyl)-1H-1,2,3-triazol-1-yl)acetamido)hexan-2-yl)ureido)pentanedioate was radiolabeled using the same oxidant system as for the free ligand and the product ¹²³I-TAAG-PSMA was obtained in 85% yield and greater than 99% radiochemical purity. The log D of the product was determined to be −3.23±0.05 (at pH 7.4).

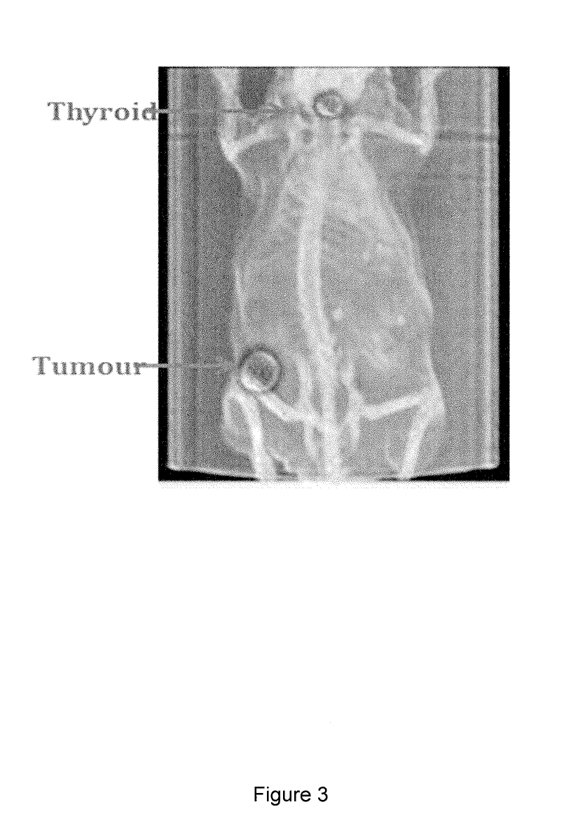

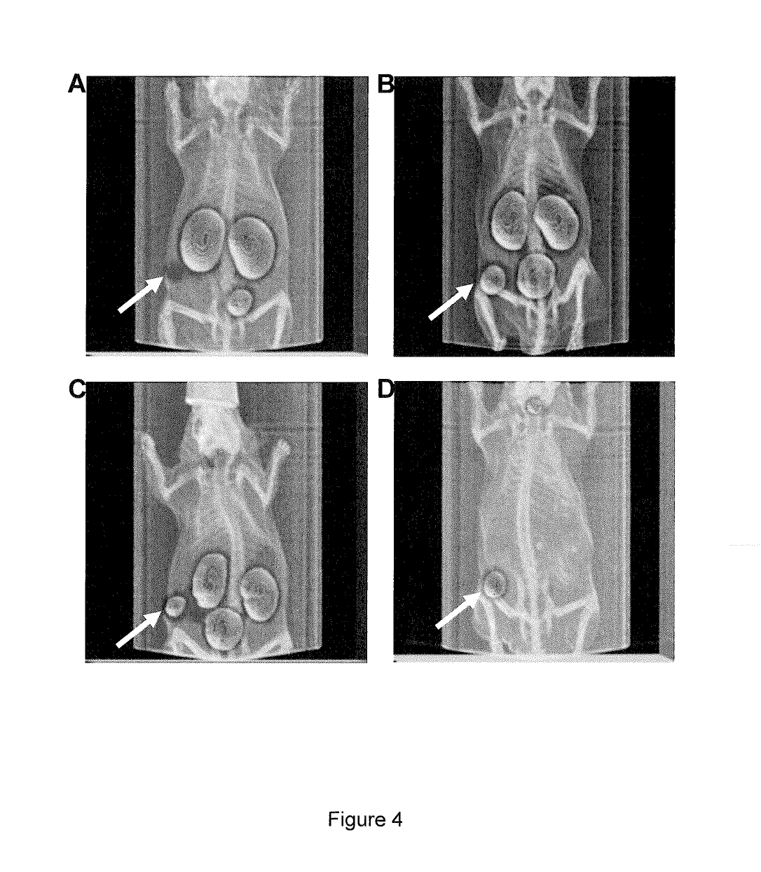

The compound ¹²³I-TAAG-PSMA was administered to NCr mice containing LNCap tumours which are known to highly express PSMA. See FIGS. 3-5. At two hours post injection, the images showed uptake of the agent in the kidneys, bladder and a small amount in the tumour. At 23-24 hours the activity was found only in the tumour and in the thyroid (FIGS. 3 and 4D). The thyroid uptake, which was higher than for ¹²³I-TAAG, is, while not wishing to be limited by theory, likely due to catabolism of the agent after prolonged retention in vivo.¹¹

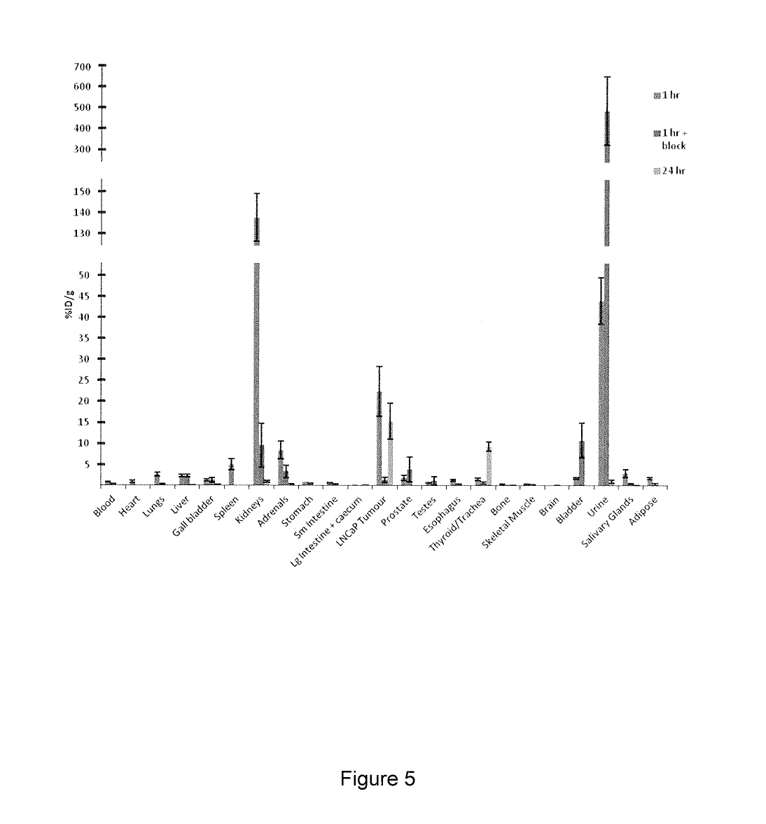

Quantitative biodistribution studies showed that tumour uptake was over 20% ID/g at 1 hour which could be blocked to less than 2% ID/g by administering a known PSMA blocking agent (phosphonomethyl) pentanedioic acid (PMPA). At 23 hours, the agent still retained 15% ID/g in the tumour and the remaining activity was in the thyroid (about 10% ID/g). See FIG. 5.

Further to this, the TAAG ester core has also been derivatized with targeting vectors comprising tertiary amines capable of binding melanin such as the N-(2-diethylaminoethyl)acetamide (TAAG-DEED) derivative and an N-benzylamino piperadine TAAG derivative, for targeting melanoma.

The TAAG synthon is versatile in that it can be prepared by taking a tin-alkyne and combining it with a suitable azide providing a scope of use that is analogous to ¹⁸F-alkynes used to develop PET agents using click-type chemistry.¹² TAAG conjugates can be, for example readily isolated, fully characterized and/or labeled using robust single step iodination methods and the products purified by HPLC or SPE. The results of the present studies demonstrate that the TAAG group promotes minimal non-specific binding and that labeled conjugates can achieve high tumor uptake and produce useful target-to-non-target ratios. The use of TAAG and functionalized analogues provides an alternative to conventional benzene-derived prosthetic groups which are useful for the development of radioiodine-based theranostics.

### Example 2

### Amino-Triazole Appending Agents (Amino-TAAG)

Amino-TAAG derivatives have been prepared which provide a second site useful for biomolecule or fluorophore derivatization.

### General Methods

All chemicals and reagents for synthesis were purchased from Sigma-Aldrich. Compounds 2-(3-(tributylstannyl)prop-2-ynyl)isoindoline-1,3-dione (method B), (3-bromoprop-1-yn-1-yl)tributylstannane and t-butyl-protected glutamate-urea-lysine were prepared according to literature procedures.^{13,14,15} Solvents were purchased from Caledon and dried using a Pure-Solv drying apparatus (Innovative Technology). ¹H, ¹³C and ¹¹⁹Sn NMR spectra were measured on a Bruker Avance AV-600 spectrometer (¹H=600.13 MHz, ¹³C=150.90 MHz, ¹¹⁹Sn=225 MHz). ¹H NMR, ¹¹⁹Sn NMR and ¹³C NMR chemical shifts are expressed in parts per million (ppm, δ units), and coupling constants are expressed in Hertz (Hz). IR spectra were recorded on a Nicolet 6700 FT-IR spectrometer (KBr disc). Low-resolution mass spectra were obtained on an Agilent 630 ion trap electron spray ionization (ESI) instrument, using a 1200 series LC system and an eluent of H₂O:MeOH (1:1). High-resolution mass spectra (HRMS) were obtained using a Waters Micromass Global Ultima Q-TOF in ESI mode. HPLC (analytical and semi-preparative) was performed on a Waters 1525 Binary HPLC system connected to a Bioscan γ-detector and a 2998 photodiode array detector monitoring at 254 nm. For analysis of compounds, a Phenomenex Gemini column (5 μm, 4.6×250 mm, C18) was used, eluting at a flow rate of 1.0 mL/min. For semi-preparative HPLC, a Phenomenex Luna column (5 μm, 10.0×250 mm, C18) was used, eluting at a flow rate of 4.0 mL/min. HPLC protocols were as follows: Analytical and semi-preparative HPLC: Solvent A=0.1% triethylamine (TEA) in water; Solvent B=0.1% TEA in acetonitrile: gradient elution, 2% B (0-8 min), 39% B (8-20 min), 2% B (21-22 min).

### Synthesis

### Preparation of 2-(3-(tributylstannyl)prop-2-ynyl)isoindoline-1,3-dione

I. Method A

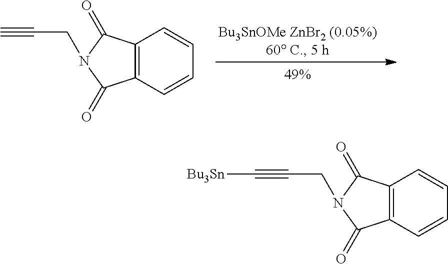

Bu₃SnOMe (345 μL, 1.2 mmol) was added dropwise to a solution of ZnBr₂ (11 mg, 0.05 mmol) and N-propargyl phthalimide (185 mg, 1 mmol) in THF (0.5 mL). The mixture was stirred for 3 h at 60° C., and then quenched with water (10 mL). The mixture was extracted with diethyl ether (3×10 mL). The combined organic extracts were dried over MgSO₄ and the solvent removed in vacuo to give the crude product. The crude product was diluted with ethyl acetate (30 mL) and washed with NH₄F_{(aq)} (10%, 20 mL). The white precipitate obtained was filtered off, and the filtrate dried over MgSO₄. The solvent was removed in vacuo to give an oily white product. Purification of the crude product by preparative TLC eluting with dichloromethane gave 2-(3-(tributylstannyl)prop-2-ynyl)isoindoline-1,3-dione (R_{f}=0.71) as a colorless oil (235 mg, 49% yield).

II. Method B

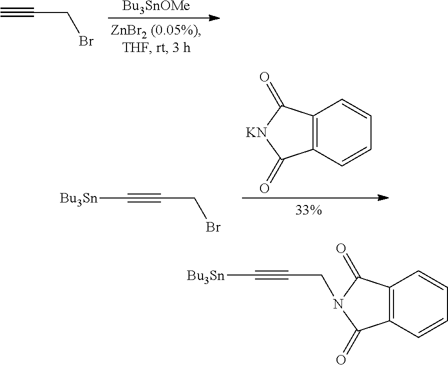

(3-Bromoprop-1-yn-1-yl)tributylstannane was prepared according to the literature¹³ then treated with potassium phthalimide as described previously¹⁴ to give 2-(3-(tributylstannyl)prop-2-ynyl)isoindoline-1,3-dione in a 33% yield.

¹H NMR: (600 MHz, CDCl₃) 7.89 (m, 2H), 7.74 (m, 2H) 4.48 (s, 2H), 1.53 (m, 6H), 1.31 (m, 6H), 1.31 (m, 6H) 0.97 (m, 6H), 0.87 (m, 9H); ¹³C NMR: (150 MHz, CDCl₃) 167.1, 134.0, 132.2, 123.4, 103.1, 86.7, 28.8, 28.2, 26.9, 13.6, 11.0; ¹¹⁹Sn NMR: (225 MHz, CDCl₃) −64.5; IR: (neat) 2955, 2159 (C≡C), 1728 cm⁻¹; HRMS (ESI⁺) m/z for C₂₃H₃₃O₂NSnH: calculated 476.1616, observed 474.1614 [M+H]⁺.

### Preparation of (S)-di-tert-butyl 2-(3-((S)-6-(2-azidoacetamido)-1-tert-butoxy-1-oxohexan-2-yl)ureido)pentanedioate

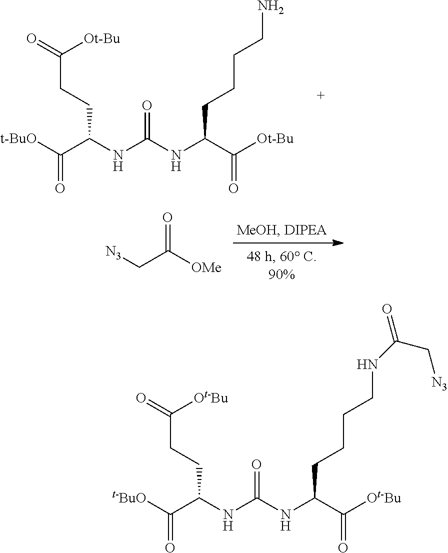 [SMILES: CC(C)(C)OC(=O)CC[C@H](NC(=O)N[C@@H](CCCCN)C(=O)OC(C)(C)C)C(=O)OC(C)(C)C ; COC(=O)CN=[N+]=[N-] ; CC(C)(C)OC(=O)CC[C@H](NC(=O)N[C@@H](CCCCNC(=O)CN=[N+]=[N-])C(=O)OC(C)(C)C)C(=O)OC(C)(C)C]

To a solution of methyl 2-azidoacetate (0.11 mL, 1.13 mmol) in MeOH (4 mL) was added t-butyl-protected glutamate-urea-lysine¹⁵ (0.650 g, 1.33 mmol) followed by DIPEA (0.200 mL, 1.15 mmol). The reaction was stirred at 60° C. for 48 h. The reaction mixture was then evaporated to dryness and purified by silica gel chromatography eluting with ethyl acetate/hexane (2:1) to provide (S)-di-tert-butyl-2-(3-((S)-6-(2-azidoacetamido)-1-tert-butoxy-1-oxohexan-2-yl)ureido)pentanedioate as a white solid (0.684 g, 90%, R_{f}=0.56).

¹H NMR: (600 MHz, CDCl₃) 7.12 (m, 1H, NH), 5.77 (d, J=6 Hz, 1H, NH), 5.51 (d, J=6 Hz, 1H, NH), 4.26 (m, H, CH), 4.13 (m, H, CH), 3.86 (2, 2H, CH₂), 3.26 (m, H, CH), 3.13 (m, H, CH), 2.24 (m, 2H, CH₂), 1.98 (m, H, CH), 1.77 (m, 1H, CH), 1.65 (m, 1H, CH), 1.38 (m, 2H, CH₂), 1.35 (s, 27H, 9CH₃), 1.25 (m, 2H, CH₂); ¹³C NMR: (150 MHz, CDCl₃) 173.3, 172.4, 172.2, 167.3, 157.4, 82.3, 81.5, 80.6, 64.2, 123.4, 53.5, 52.9, 52.3, 39.1, 32.4, 28.6, 28.1, 27.9, 25.3, 22.9; IR: (neat) 3315, 2976, 2925, 1733 cm⁻¹; HRMS (ESI⁺) m/z for C₂₆H₄₆O₈N₆H: calculated 571.3455, observed 571.3459 [M+H]⁺.

### Preparation of (S)-di-tert-butyl 2-(3-((S)-1-tert-butoxy-6-(2-(5-((1,3-dioxoisoindolin-2-yl)methyl)-4-(tributylstannyl)-1H-1,2,3-triazol-1-yl)acetamido)-1-oxohexan-2-yl)ureido)pentanedioate

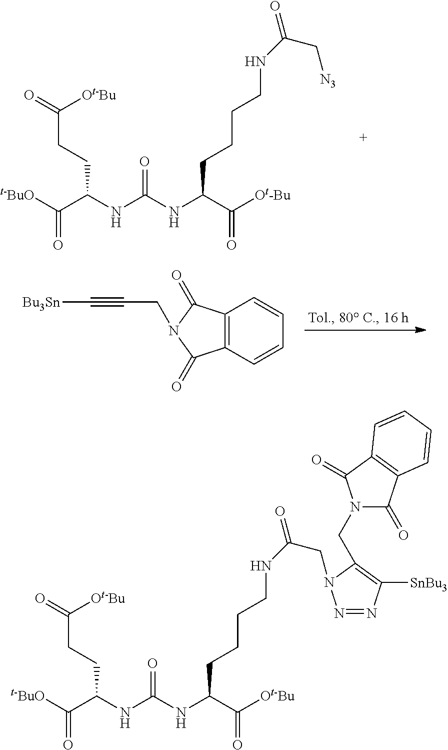

(3-Bromoprop-1-yn-1-yl)tributylstannane (0.362 g, 0.76 mmol) was dissolved in toluene (5 mL) followed by the addition of (S)-di-tert-butyl-2-(3-((S)-6-(2-azidoacetamido)-1-tert-butoxy-1-oxohexan-2-yl)ureido)pentanedioate (0.300 g, 0.53 mmol). The reaction mixture was stirred at 80° C. for 16 h. The reaction was then cooled to room temperature, concentrated and purified twice by prep-TLC using ethyl acetate/hexane (2:1) as eluent (R_{f}=0.50) where the product was isolated as a colorless oil (0.275 g) in 50% yield.

¹H NMR: (600 MHz, CDCl₃) 7.83 (m, 2H, 2CH_{Arom.}), 7.72 (m, 2H, 2CH_{Arom.}), 6.99 (t, J=6 Hz, 1H, NH), 5.87 (d, J=12 Hz, 1H, NH), 5.56 (d, J=6 Hz, 1H, NH), 5.35 (s, 2H, CH₂), 5.05 (dd, J=6 Hz, 2H, CH₂), 4.36 (m, H, CH), 4.31 (m, H, CH), 2.34 (m, 2H, CH₂), 2.07 (m, H, CH), 1.84 (m, H, CH), 1.74 (m, 1H, CH), 1.55-1.41 (m, 29H, 9CH₃+CH₂), 1.31 (m, 6H, 3CH₂), 1.24 (m, 4H, 2CH₂), 0.87 (t, J=6 Hz, 9H, 3CH₃); ¹³C NMR: (150 MHz, CD₃CN) 172.1, 171.9, 171.8, 171.6, 167.5, 166.9, 165.4, 157.0, 145.9, 139.2, 134.1, 131.6, 122.8, 80.9, 80.6, 79.7, 53.2, 52.5, 51.3, 49.8, 38.3, 38.2, 31.5, 31.4, 30.9, 30.6, 28.6, 28.5, 28.4, 27.9, 27.5, 26.9, 22.1, 21.9, 12.7, 9.5; ¹¹⁹Sn NMR: (225 MHz, CD₃CN) −58.8; IR: (neat) 3331, 2923, 1700, 1154 cm⁻¹; HRMS (ESI⁺) m/z for C₄₉H₇₉O₁₀N₇SnH: calculated 1046.5001, observed 1046.5026 [M+H]⁺.

### Preparation of di-tert-butyl 2-(3-(6-(2-(5-(aminomethyl)-4-(tributylstannyl)-1H-1,2,3-triazol-1-yl)acetamido)-1-tert-butoxy-1-oxohexan-2-yl)ureido)pentanedioate

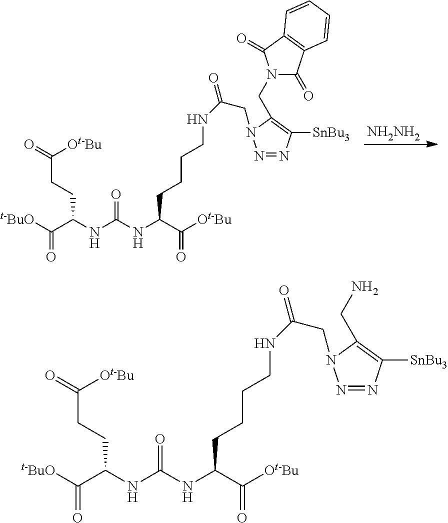

Hydrazine hydrate (70 mg, 1.46 mmol) was added to a stirred solution of the phthalimide (148 mg, 0.143 mmol), and the solution was heated to reflux for five hours. It was then cooled to room temperature, and the solvent was removed under reduced pressure. The residue was dissolved in NaOH (3 mL, 20%), and the resulting two layers were then extracted with CH₂Cl₂ (3×5 mL). The combined organic solution was then dried over MgSO₄. The filtrate was evaporated to leave the product amine as a colorless oil (70 mg) in 54% yield.

¹H NMR: (600 MHz, CDCl₃) 7.17 (t, J=6 Hz, 1H, NH), 5.83 (d, J=12 Hz, 1H, NH), 5.48 (d, J=6 Hz, 1H, NH), 5.18 (dd, 2H, CH₂), 4.33 (m, 1H, CH), 4.23 (m, 1H, CH), 3.92 (s, 2H, CH₂), 3.29 (m, 1H, CH), 3.10 (m, 1H, CH), 2.31 (m, 2H, CH₂), 2.04 (m, 1H, CH), 1.83 (m, 2H), 1.71 (m, 2H), 1.51 (m, 6H), 1.44 (m, 1H), 1.40 (m, 12H), 1.39 (m, 11H), 1.29 (m, 6H), 1.12 (m, 5H), 0.86 (t, J=6 Hz, 9H, 3CH₃); ¹³C NMR: (150 MHz, CDCl₃) 173.2, 172.4, 172.3, 167.4, 166.4, 157.7, 144.8, 144.3, 82.2, 81.5, 80.6, 53.3, 53.2, 52.4, 51.0, 0.8, 39.0, 35.5, 31.8, 29.0, 28.4, 28.1, 28.0, 27.3, 22.1, 27.9, 27.5, 26.9, 22.1, 21.9, 13.7, 10.1; ¹¹⁹Sn NMR: (225 MHz, CDCl₃) −59.7; IR: (neat) 3314, 2956, 1732, 1155 cm⁻¹; HRMS (ESI⁺) m/z for C₄₁H₇₇O₈N₇SnH: calculated 916.4943, observed 916.4943 [M+H]⁺.

### Preparation of 1-Amino-TAAG-PSMA (cold standard)

### Preparation of (R)-di-tert-butyl 2-(3-((R)-1-tert-butoxy-6-(2-(5-((1,3-dioxoisoindolin-2-yl)methyl)-4-iodo-1H-1,2,3-triazol-1-yl)acetamido)-1-oxohexan-2-yl)ureido)pentanedioate

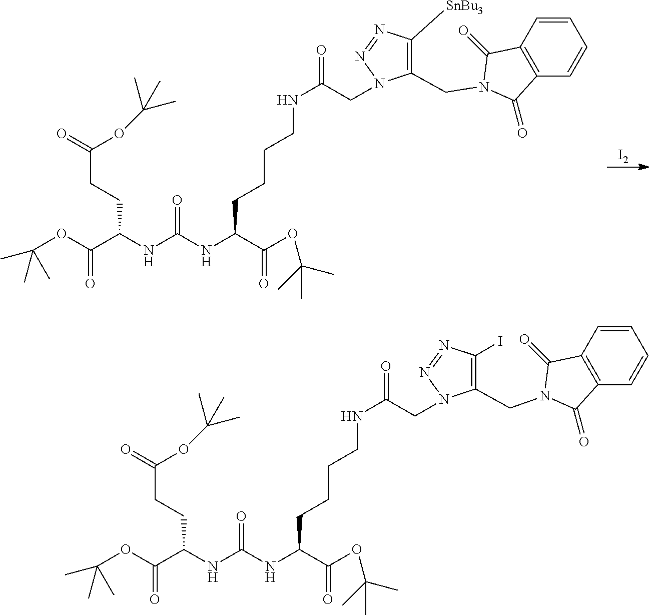

To a solution of (S)-di-tert-butyl 2-(3-((S)-1-tert-butoxy-6-(2-(5-((1,3-dioxoisoindolin-2-yl)methyl)-4-(tributylstannyl)-1H-1,2,3-triazol-1-yl)acetamido)-1-oxohexan-2-yl)ureido)pentanedioate (240 mg, 2.23 mmol) in DCM (5 mL), I₂ (64 mg, 0.256 mmol) was added at room temperature and the mixture was stirred for 15 min. The reaction was passed through a silica plug (2 g, 10 wt % KF mixed in silica) and concentrated under vacuum to dryness and purified by silica gel chromatography using hexane/ethyl acetate as eluents (1:2) to provide the iodinated product as a colorless oil (180 mg) in 89% yield.

¹H NMR: (600 MHz, CDCl₃) 7.85 (m, 2H, 2CH_{Arom.}), 7.74 (m, 2H, 2CH_{Arom.}), 5.79 (m, 1H, NH), 5.47 (m, 3H, NH, CH₂), 5.02 (t, J=12 Hz, 2H, CH₂), 4.37 (m, 1H, CH), 4.30 (m, H, CH), 3.29 (m, H, CH), 3.08 (m, 1H, CH), 2.34 (m, 2H, CH₂), 2.08 (m, 1H, CH), 1.89 (m, 2H, CH₂), 1.75 (m, 1H, CH), 1.56 (m, 2H, CH₂), 1.47 (s, 9H, 3CH₃), 1.43 (s, 9H, 3CH₃), 1.41 (s, 9H, 3CH₃), 1.32 (m, 2H, CH₂); ¹³C NMR: (150 MHz, CDCl₃) 174.0, 172.5, 172.3, 167.8, 165.4, 157.6, 135.9, 134.5, 131.9, 123.9, 91.6, 82.4, 81.8, 80.8, 53.6, 53.4, 53.2, 52.6, 52.0, 39.4, 32.6, 32.1, 31.9, 30.8, 28.8, 28.2, 28.0, 22.7, 22.2; IR: (neat) 3344, 2975, 2928, 1712 cm⁻¹; HRMS (ESI⁺) m/z for C₃₇H₅₂IO₁₀N₇H: calculated 882.2899, observed 882.2899 [M+H]⁺.

### Preparation of (R)-di-tert-butyl 2-(3-((R)-6-(2-(5-(aminomethyl)-4-iodo-1H-1,2,3-triazol-1-yl)acetamido)-1-tert-butoxy-1-oxohexan-2-yl)ureido)pentanedioate

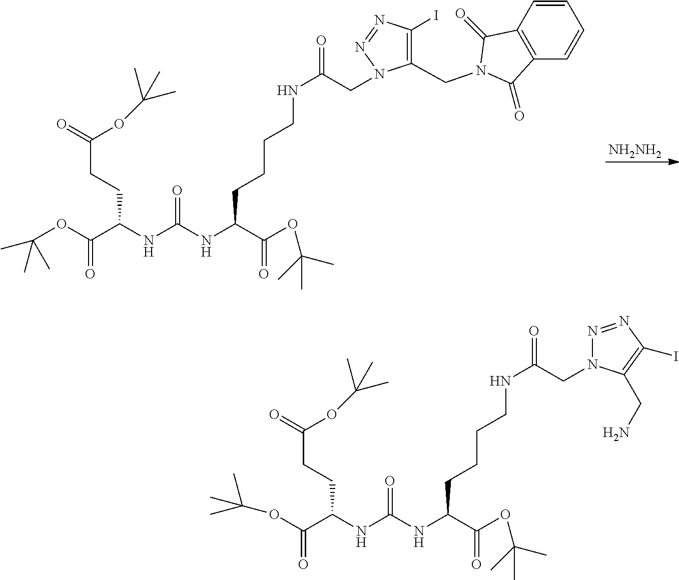 [SMILES: CC(C)(C)OC(=O)CC[C@H](NC(=O)N[C@@H](CCCCNC(=O)Cn1nnc(I)c1CN1C(=O)c2ccccc2C1=O)C(=O)OC(C)(C)C)C(=O)OC(C)(C)C ; "NH2NH2" ; CC(C)(C)OC(=O)CC[C@H](NC(=O)N[C@@H](CCCCNC(=O)Cn1nnc(I)c1CN)C(=O)OC(C)(C)C)C(=O)OC(C)(C)C]

Hydrazine hydrate (103 mg, 2.08 mmol) was added to a stirred solution of the phthalimide (180 mg, 0.204 mmol), and the solution was heated to reflux for five hours. It was then cooled to room temperature, and the solvent was removed under reduced pressure. The residue was dissolved in NaOH (3 mL, 20%), and the resulting two layers were then extracted with CH₂Cl₂ (3×5 mL). The combined organic solution was then dried over MgSO₄. The filtrate was evaporated to leave the product amine as a colorless oil (92 mg) in 60% yield.

¹H NMR: (600 MHz, CDCl₃) 7.65 (t, J=6 Hz, 1H, NH), 5.94 (d, J=6 Hz, 1H, NH), 5.61 (d, J=6 Hz, 1H, NH), 5.28 (dd, 2H, CH₂), 4.35 (m, 1H, CH), 4.27 (m, 1H, CH), 3.94 (s, 2H, CH₂), 3.35 (m, 1H, CH), 3.19 (m, 1H, CH), 2.35 (m, 2H, CH₂), 2.07-2.03 (m, 5H), 1.88 (m, 1H), 1.75 (m, 1H), 1.58 (m, 3H), 1.47 (s, 9H, 3CH₃), 1.45 (s, 9H, 3CH₃), 1.43 (s, 9H, 3CH₃), 1.35 (m, 2H); ¹³C NMR: (150 MHz, CDCl₃) 173.5, 172.5, 172.3, 165.6, 157.6, 141.0, 89.2, 82.4, 81.7, 80.7, 53.4, 53.3, 51.6, 50.6, 39.3, 35.2, 32.3, 31.7, 28.3, 28.1, 27.9, 22.5; IR: (neat) 3289, 2925, 1735, 1615 cm⁻¹, HRMS (ESI⁺) m/z for C₂₉H₅₀IO₈N₇H: calculated 752.2844, observed 752.2842 [M+H]⁺.

### Preparation of (R)-2-(3-((R)-5-(2-(5-(aminomethyl)-4-iodo-1H-1,2,3-triazol-1-yl)acetamido)-1-carboxypentyl)ureido)pentanedioic acid

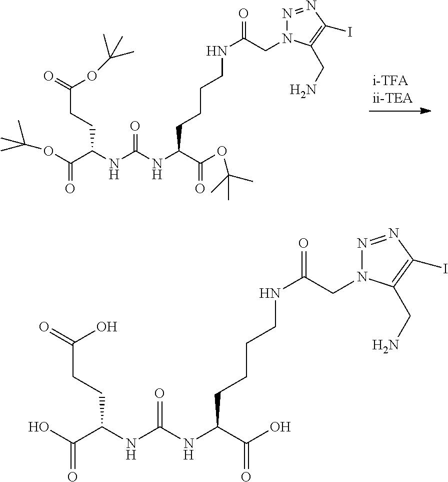 [SMILES: CC(C)(C)OC(=O)CC[C@H](NC(=O)N[C@@H](CCCCNC(=O)Cn1nnc(I)c1CN)C(=O)OC(C)(C)C)C(=O)OC(C)(C)C ; NCc1c(I)nnn1CC(=O)NCCCC[C@H](NC(=O)N[C@@H](CCC(=O)O)C(=O)O)C(=O)O]

(R)-di-tert-butyl 2-(3-((R)-6-(2-(5-(aminomethyl)-4-iodo-1H-1,2,3-triazol-1-yl)acetamido)-1-tert-butoxy-1-oxohexan-2-yl)ureido)pentanedioate (30 mg, 3.99 mmol) was dissolved in 1:1 (v/v) TFA:DCM (3 mL) and stirred at room temperature for 24 h. Evaporation of volatiles gave the crude product as a TFA salt which was dissolved in water, lyophilised and analyzed by NMR (white solid, 17 mg, 74% yield). Free amine was obtained by dissolving the TFA salt in MeOH (1 mL) and treating it with TEA (triethanolamine) (100 μL) at room temperature for 1 hour. The reaction mixture was concentrated, purified by semi-preparative HPLC and lyophilised to provide the product as a white paste (tᵣ=6.05 min).

¹H NMR: (600 MHz, DMSO-d₆) 12.48 (bs, 3H, 3COOH), 8.81 (s, 1H, NH), 8.28 (bs, 3H, NH₃), 6.37-6.32 (m, 2H, 2NH), 5.34 (s, 2H, CH₂), 4.20 (s, 2H, CH₂), 4.13 (m, 2H, 2CH), 3.12 (m, 2H, CH₂), 2.25 (m, 2H, CH₂), 1.93-1.88 (m, 1H), 1.75-1.65 (m, 2H), 1.56-1.44 (m, 3H), 1.33-1.24 (m, 2H); ¹³C NMR: (150 MHz, DMSO-d₆) 174.5, 174.2, 173.7, 165.7, 157.4, 134.8, 95.4, 52.2, 51.7, 51.2, 31.7, 30.8, 29.9, 28.3, 28.0, 27.4, 22.6; IR: (neat) 3314, 2959, 1684, 1135 cm⁻¹; HRMS (ESI⁺) m/z for C₁₇H₂₅IO₈N₇: calculated 582.0809, observed 582.0809 [M+H]⁻.

### di-tert-butyl 2-(3-(1-tert-butoxy-6-(2-(5-((2,4-dinitrophenylamino)methyl)-4-iodo-1H-1,2,3-triazol-1-yl)acetamido)-1-oxohexan-2-yl)ureido)pentanedioate

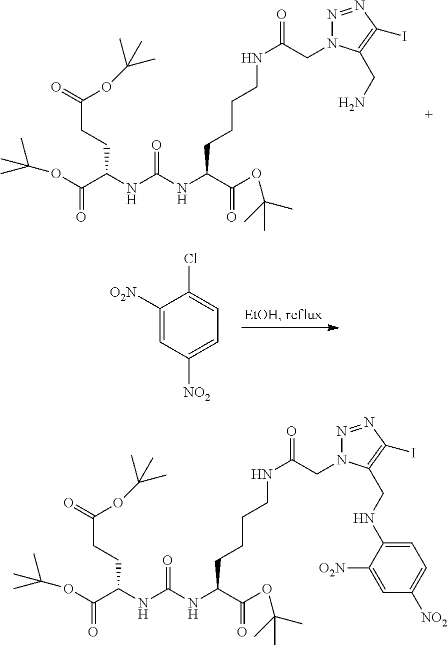 [SMILES: CC(C)(C)OC(=O)CC[C@H](NC(=O)N[C@@H](CCCCNC(=O)Cn1nnc(I)c1CN)C(=O)OC(C)(C)C)C(=O)OC(C)(C)C ; O=[N+]([O-])c1ccc(Cl)c([N+](=O)[O-])c1 ; CC(C)(C)OC(=O)CC[C@H](NC(=O)N[C@@H](CCCCNC(=O)Cn1nnc(I)c1CNc1ccc([N+](=O)[O-])cc1[N+](=O)[O-])C(=O)OC(C)(C)C)C(=O)OC(C)(C)C]

Into a flask were added 1-chloro-2,4-dinitrobenzene (10 mg, 4.95 mmol), (R)-di-tert-butyl 2-(3-((R)-6-(2-(5-(aminomethyl)-4-iodo-1H-1,2,3-triazol-1-yl)acetamido)-1-tert-butoxy-1-oxohexan-2-yl)ureido)pentanedioate (30 mg, 3.99 mmol), and ethanol (2 mL). The mixture was heated to reflux (8 h, changing to yellow) and concentrated under reduced pressure. Purification of the crude by preparative TLC using 9:1 DCM/MeOH (R_{f}=0.62) gave the title compound as a yellow oil (14 mg, 38% yield).

¹H NMR: (600 MHz, CDCl₃) 9.13 (bs, 1H, CH_{Arom}), 8.93 (bs, 1H, NH), 8.32 (bs, 1H, CH_{Arom}), 7.77 (bs, 1H, NH), 7.05 (bs, 1H, CH_{Arom}), 5.84 (bs, 1H, NH), 5.47 (s, 1H, NH), 5.33 (m, 1H, CH), 5.15 (m, 1H, CH), 4.89 (d, J=12, 2H, CH₂), 4.35 (bs, 1H, CH), 4.22 (bs, 1H, CH), 3.49 (s, 2H, CH₂), 3.30 (bs, 1H, CH), 3.17 (bs, 1H, CH), 2.34 (s, 2H, CH₂), 2.07 (bs, 1H, CH), 2.01 (bs, 1H, CH), 1.87 (m, 1H, CH), 1.77 (m, 2H, CH₂), 1.53-1.39 (m, 29H, CH₂+9CH₃); ¹³C NMR: (150 MHz, CDCl₃) 173.9, 172.3, 172.2, 164.9, 157.7, 147.5, 137.1, 134.9, 134.8, 131.5, 130.5, 124.0, 114.5, 90.9, 82.9, 81.9, 80.9, 53.5, 53.4, 51.8, 50.8, 39.4, 37.4, 32.4, 31.6, 29.6, 28.0, 27.9, 27.8, 27.6, 22.7; HRMS (ESI⁺) m/z for C₃₅H₅₂IO₁₂N₉H: calculated 918.2858, observed 918.2833 [M+H]⁺. IR: (neat) 3315, 3100, 2979, 1732, 1157 cm⁻¹.

### (R)-2-(3-((R)-1-carboxy-5-(2-(5-((2,4-dinitrophenylamino)methyl)-4-iodo-1H-1,2,3-triazol-1-yl)acetamido)pentyl)ureido)pentanedioic acid

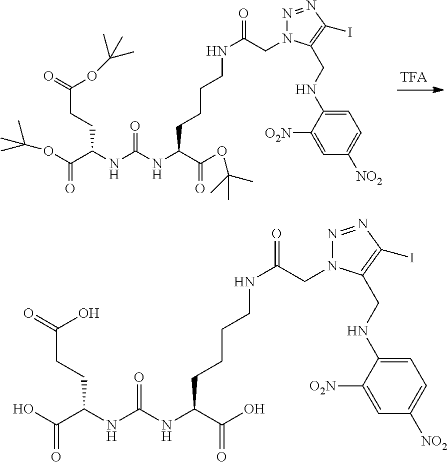 [SMILES: CC(C)(C)OC(=O)CC[C@H](NC(=O)N[C@@H](CCCCNC(=O)Cn1nnc(I)c1CNc1ccc([N+](=O)[O-])cc1[N+](=O)[O-])C(=O)OC(C)(C)C)C(=O)OC(C)(C)C ; O=C(O)CC[C@H](NC(=O)N[C@@H](CCCCNC(=O)Cn1nnc(I)c1CNc1ccc([N+](=O)[O-])cc1[N+](=O)[O-])C(=O)O)C(=O)O]

Di-tert-butyl 2-(3-(1-tert-butoxy-6-(2-(5-((2,4-dinitrophenylamino)methyl)-4-iodo-1H-1,2,3-triazol-1-yl)acetamido)-1-oxohexan-2-yl)ureido)pentanedioate (10 mg, 1.09 mmol) was dissolved in 1:1 (v/v) TFA:DCM (1 mL) and stirred at room temperature for 10 h. Evaporation of volatiles gave the crude product as a dark yellow waxy solid which was dissolved in water, and lyophilised to provide the product as a pale brown solid (7 mg, 87% yield).

¹H NMR: (600 MHz, DMSO-d₆) 12.32 (bs, 3H, 3COOH), 9.07 (t, J=6 Hz, 1H, CH_{Arom}), 8.82 (d, J=6 Hz, 1H, CH_{Arom}), 8.33 (t, J=6 Hz, 1H, NH), 8.20 (d, J=6 Hz, 1H, CH_{Arom}), 7.12 (d, J=6 Hz, 1H, NH), 6.25 (t, J=6 Hz, 2H, 2NH), 5.21 (s, 2H, CH₂), 4.78 (d, J=6, 2H, CH₂), 4.03 (m, 2H, 2CH), 2.97 (bs, 2H, CH₂), 2.19 (bs, 2H, CH₂), 1.65 (bs, 1H, CH), 1.57-1.44 (m, 1H, CH), 1.33 (s, 1H, CH), 1.22 (bs, 2H, CH₂), 1.17 (bs, 2H, CH₂), 1.10 (m, 1H, CH); ¹³C NMR: (150 MHz, DMSO-d₆) 174.6, 174.3, 173.8, 164.9, 157.3, 147.6, 147.3, 135.6, 135.5, 131.0, 130.7, 129.8, 123.3, 115.6, 91.0, 69.9, 52.2, 51.7, 50.7, 37.3, 36.6, 31.8, 29.9, 29.0, 28.4, 28.2, 27.6, 22.6; IR: (neat) 3430, 3272, 2253, 1653, 1027 cm⁻¹; HRMS (ESI⁺) m/z for C₂₃H₂₇IO₁₂N₉: calculated 748.0824, observed 748.0788 [M−H]⁻.

### PSMA Binding Competition Screen for ¹²⁵I-amino-TAAG-PSMA

These are the results of 4 consecutive screens using LNCaP cells to assess binding of the ¹²⁵I-amino-TAAG-PSMA.

The screens were completed with 2 lots of ¹²⁵I-TAAG-PSMA, one was labelled 16 Jul. 2012 and purified by HPLC 17 Jul. 2012, the other was labelled 22 Aug. 2012 and purified by HPLC 23 Aug. 2012. ¹²⁵I-amino-TAAG-PSMA was dissolved in 58% DMSO/water to a stock concentration of 5 mM, aliquoted and stored at −20° C.

Table 6 summarizes the individual screen results.

Table 7 contains the analysis results of all data points combined from all experiments and analyzed as one single data set (GraphPad) and the analysis of the results from individual experiments which were then combined and averaged (Excel). The latter demonstrates variability between experiments.

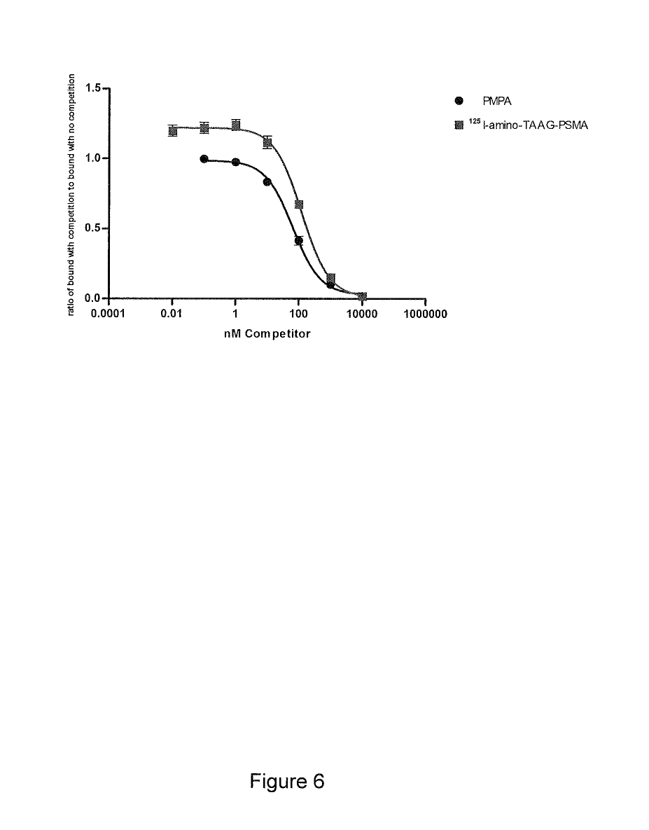

As a reference, the combined aggregate data for PMPA for these experiments resulted in an IC50 of 65.2 nM (95% Cl: 53.2, 79.8; R²: 0.9726; std error of curve fit: 1.11). FIG. 6 is a graph of all the data from all experiments combined (4 assays, n=4 each) and analyzed together (GraphPad).

### Example 3

### N-(2-diethylaminoethyl)acetamide Derivitized Triazole Appending Agents (TAAG-DEED)

### Preparation of N-(2-(diethylamino)ethyl)-2-(4-(4,4,5,5,6,6,7,7,8,8,9,9,9-tridecafluorononyl)-1H-1,2,3-triazol-1-yl)acetamide

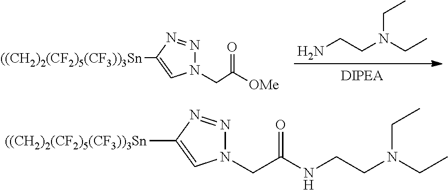

To a solution of the methyl ester (56 mg, 0.043 mmol) in methanol (8 mL), N,N′-diethylethylenediamine (8 μL, 0.06 mmol) and diisopropylethylamine (22 μL, 0.13 mmol) were added at room temperature. The reaction was stirred at 60° C. for 4 h, concentrated by rotary evaporation and purified using silica gel chromatography (30 g SiO₂, dichloromethane/methanol (10:1)) and the fractions containing only the desired product were combined and concentrated by rotary evaporation to yield the product amide (52 mg, 88%). Melting point, ¹H NMR, ¹³C NMR, IR and HRMS have been completed for the above compound and the results confirm the structure of the desired compound.

### Preparation of (4-tributylstannanyl-[1,2,3]triazol-1-yl)-acetic acid methyl ester

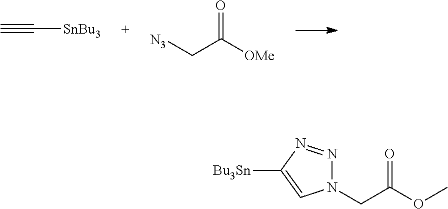

To ethynyltributylstannane (1.2 g, 3.8 mmol) in dry toluene (30 mL) was added methyl 2-azidoacetate (0.43 g, 3.8 mmol). The mixture was heated at 120° C. overnight. The reaction mixture was then concentrated by rotary evaporation and purified using silica gel chromatography (50 g SiO₂, hexanes/ethyl acetate (3:1)) and the fractions containing only the desired products concentrated by rotary evaporation to yield the triazole (1.00 g, 61%). Melting point, ¹H NMR, ¹³C NMR, IR and HRMS have been completed for the above compound and the results confirm the structure of the desired compound.

### Preparation of N-(2-Diethylamino-ethyl)-2-(4-tributylstannanyl-[1,2,3]triazol-1-yl)acetamide

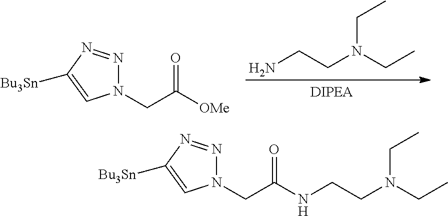

To a solution of the methyl ester (0.89 g, 1.9 mmol) in methanol (3 mL), N,N′-diethylethylenediamine (0.82 mL, 5.8 mmol) and diisopropylethylamine (1.0 mL, 5.8 mmol) were added. The mixture was heated at 60° C. overnight. The reaction mixture was then concentrated by rotary evaporation and purified by silica gel chromatography (25 g SiO₂, dichloromethane/methanol (6:1)). The fractions containing only the desired product were concentrated by rotary evaporation to yield the amide (0.89 g, 89%). Melting point, ¹H NMR, ¹³C NMR, IR and HRMS have been completed for the above compound and the results confirm the structure of the desired compound.

### Preparation of N-(2-Diethylamino-ethyl)-2-(4-iodo-[1,2,3]triazol-1-yl)-acetamide

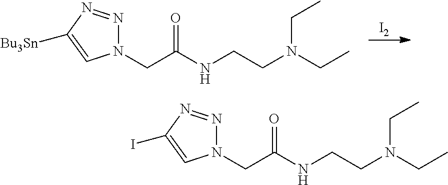

To a solution of the tributyltin triazole (71 mg, 0.14 mmol) in dry THF (3 mL) at room temperature, I₂ (45 mg, 0.18 mmol) was added. The solution turned dark brown/purple and was left to stir at room temperature for 10 min. The solution was then concentrated by rotary evaporation, and purified by silica gel chromatography (40 g SiO₂, dichloromethane/methanol (4:1)). The fractions containing only the desired product were concentrated by rotary evaporation to yield the iodo compound (20 mg, 41%). Melting point, ¹H NMR, ¹³C NMR, IR and HRMS have been completed for the above compound and the results confirm the structure of the desired compound.

### Preparation of ¹²⁵I labelled N-(2-Diethylamino-ethyl)-2-(4-iodo-[1,2,3]triazol-1-yl)-acetamide

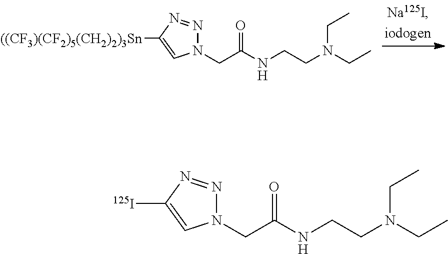

To a solution of the fluorostannylated triazole (100 μg, 0.0721 μmol) in methanol (100 μL), iodogen (25 μg), glacial acetic acid (20 μL), and Na¹²⁵I (224 μCi) were added. The reaction was left for 10 minutes with occasional shaking. To the reaction mixture, sodium metabisulphate (100 μL, 10 μmol) was added and the solution diluted to 1.5 mL with distilled water. The reaction mixture was loaded on a fluoroflash solid phase extraction cartridge, which had been activated with DMF (1 mL), followed by 80/20 methanol water (5 mL). The cartridge was washed with water (3 mL), 80/20 water:methanol (11 mL), 50/50 water:methanol (3 mL), 80/20 of methanol:water (7 mL), methanol (8 mL), and acetonitrile (1% trifluoroacetic acid, 13 mL). The radiochemical yield of the reaction was 32%. HPLC Rt=6.05 min.

### Preparation of ¹²³I labelled N-(2-Diethylamino-ethyl)-2-(4-iodo-[1,2,3]triazol-1-yl)-acetamide

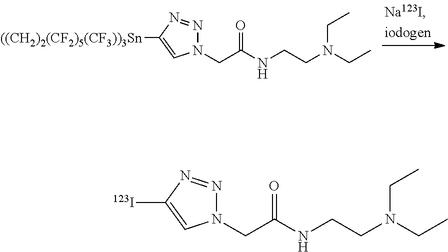

To a solution of the fluorostannylated triazole (200 μg, 0.144 μmol) in methanol (100 μL), iodogen (25 μg), glacial acetic acid (26 μL), and Na¹²³I (7.31 mCi) were added. The reaction was left for 10 min with occasional shaking to yield the ¹²³I-labelled triazole. To the reaction mixture sodium thiosulfate (100 μL, 10 μmol) was added, the crude reaction mixture was diluted to 1.5 mL with water. The reaction mixture was loaded on a fluoroflash solid phase extraction cartridge, which had been activated with DMF (1 mL) followed by 80/20 methanol water (5 mL). The cartridge was washed with water (12 mL), 80/20 water:methanol (22 mL), 50/50 water:methanol (12 mL), 80/20 of methanol:water (14 mL), methanol (16 mL), and acetonitrile (1% trifluoroacetic acid, 10 mL). The radiochemical yield of the reaction was 18%. HPLC Rt=6.05 min.

### Example 4

### Phenyl-Triazole Appending Agents

### Preparation of phenylethynyl-tris-(3,3,4,4,5,5,6,6,7,7,8,8,8-tridecafluoro-octyl)-stannane

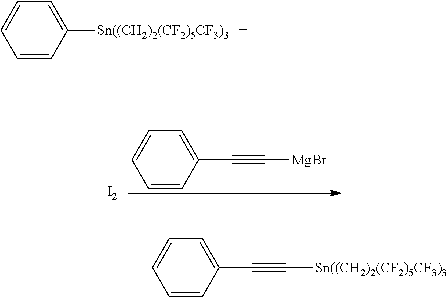

To a solution of tris(1H,1H,2H,2H-perfluorooctyl)phenyltin (1.2 g, 0.99 mmol) in dry THF, I₂ (0.30 g, 1.2 mmol) was added and the reaction was allowed to stir at room temperature for 30 min, at which point phenylethynylmagnesium bromide (5.4 mL, 5.4 mmol) was added dropwise over one minute. The reaction was stirred at room temperature for six hours at which point it was extracted with FC-72 (3×25 mL). The FC-72 layer was combined and dried over sodium sulfate and concentrated by rotary evaporation to yield the alkyne, which was used in the next step without further purification. Melting point, ¹H NMR, ¹³C NMR, IR and HRMS have been completed for the above compound, and the results confirm the structure of the desired compound.

### Preparation of {5-Phenyl-4-[tris-(3,3,4,4,5,5,6,6,7,7,8,8,8-tridecafluoro-octyl)-stannanyl]-[1,2,3]triazol-1-yl}-acetic acid methyl ester

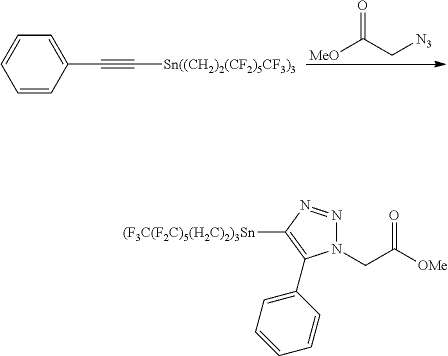

To the alkyne in dry toluene (30 mL) was added methyl 2-azidoacetate (0.17 g, 1.5 mmol), and the mixture was stirred at 120° C. overnight. The reaction was concentrated by rotary evaporation and purified using silica gel chromatography (40 g SiO₂, hexanes/ethyl acetate (4:1)). The fractions containing only the desired product were concentrated by rotary evaporation to yield the phenyl substituted triazole (0.21 g, 13%). Melting point, ¹H NMR, ¹³C NMR, IR and HRMS have been completed for the above compound, and the results confirm the structure of the desired compound.

### Preparation of N-(2-Diethylamino-ethyl)-2-{5-phenyl-4-[tris-(3,3,4,4,5,5,6,6,7,7,8,8,8-tridecafluoro-octyl)-stannanyl]-[1,2,3]triazol-1-yl}-acetamide

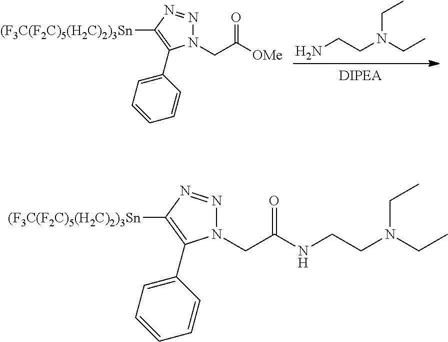

To a solution of the ester (90 mg, 0.064 mmol) in 3 mL of methanol, N,N′-diethylethylenediamine (0.30 mL, 2.2 mmol) and diisopropylethylamine (0.38 mL, 2.2 mmol) were added. The mixture was stirred at 60° C. overnight. The reaction mixture was then concentrated by rotary evaporation and purified by silica gel chromatography (40 g SiO₂, dichloromethane/methanol (8:1)). The fractions containing only the desired product were concentrated by rotary evaporation to yield the amide. Melting point, ¹H NMR, ¹³C NMR, IR and HRMS have been completed for the above compound, and the results confirm the structure of the desired compound.

### Preparation of (5-Phenyl-4-tributylstannanyl-[1,2,3]triazol-1-yl)-acetic acid methyl ester

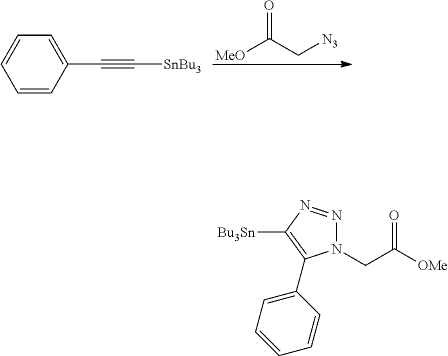

To tributyl(phenylethynyl)stannane (2.0 g, 5.2 mmol) in dry toluene (30 mL), was added methyl 2-azidoacetate (0.78 g, 6.8 mmol), and the mixture was heated at 120° C. overnight. The reaction mixture was then concentrated by rotary evaporation and purified using silica gel chromatography (40 g SiO₂, hexanes/ethyl acetate (2:1)). The fractions containing only the desired product were concentrated by rotary evaporation to yield the triazole (0.20 g, 8%). Melting point, ¹H NMR, ¹³C NMR, IR and HRMS have been completed for the above compound, and the results confirm the structure of the desired compound.

### Preparation of N-(2-Diethylamino-ethyl)-2-(5-phenyl-4-tributylstannanyl-[1,2,3]triazol-1-yl) acetamide

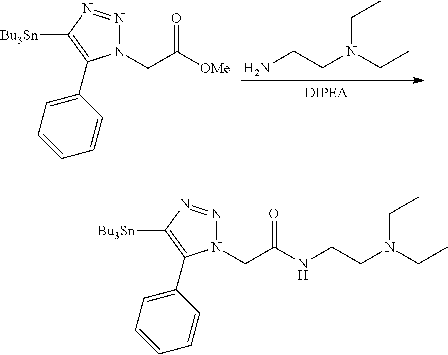

To a solution of the methyl ester (0.20 g, 0.40 mmol) in methanol (3 mL), N,N′-diethylethylenediamine (0.17 mL, 1.2 mmol) and diisopropylethylamine (0.21 mL, 1.2 mmol) were added. The mixture was heated at 60° C. overnight. The reaction mixture was then concentrated by rotary evaporation and purified by silica gel chromatography (40 g SiO₂, dichloromethane/methanol (5:1)). The fractions containing only the desired product were concentrated by rotary evaporation to yield the amide (0.20 g, 83%). Melting point, ¹H NMR, ¹³C NMR, IR and HRMS have been completed for the above compound, and the results confirm the structure of the desired compound.

### Preparation of N-(2-Diethylamino-ethyl)-2-(4-iodo-[1,2,3]triazol-1-yl)-acetamide

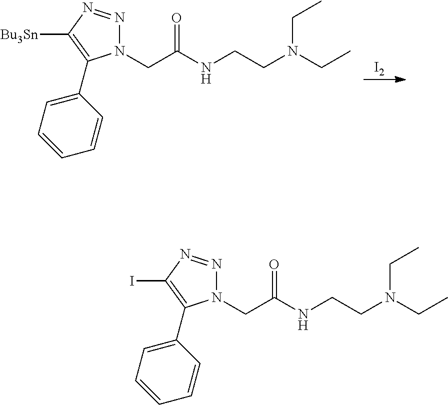

To a solution of the tributyltin triazole (0.10 g, 0.17 mmol) in dry THF (3 mL) was added I₂ (51 mg, 0.20 mmol), and the reaction was allowed to stir for 10 min at which point it was concentrated by rotary evaporation and purified by silica gel chromatography (40 g SiO₂, dichloromethane/methanol (8:1)). The iodotriazole was isolated as a clear oil (10 mg, 14%). Melting point, ¹H NMR, ¹³C NMR, IR and HRMS have been completed for the above compound, and the results confirm the structure of the desired compound.

### Preparation of ¹²⁵I labelled N-(2-Diethylamino-ethyl)-2-(4-iodo-[1,2,3]triazol-1-yl)-acetamide

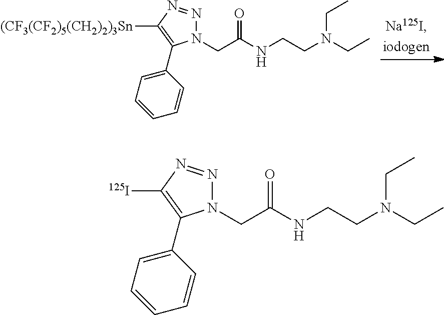

To a solution of the fluorostannylated triazole (100 μg, 0.234 μmol) in methanol (100 μL), iodogen (25.0 μg), glacial acetic acid (5 μL), and Na¹²⁵I (647 μCi) were added. The reaction was left for 10 min with occasional shaking to yield the ¹²⁵I-labelled triazole. To this was added 0.1 M sodium metabisulphate (100 μL) and the mixture was agitated for 1 min. The reaction mixture was purified by HPLC using a gradient method of H₂O+0.1% trifluoroacetic acid, and ACN+0.1% trifluoroacetic acid. RCY 17%, RCP>99%. HPLC Rt=9.85 min.

### Example 5

### Piperidinyl Derivatized Triazole Appending Agents

### Preparation of N-(1-benzylpiperidin-4-yl)-2-(4-(tributylstannyl)-1H-1,2,3-triazol-1-yl)acetamide

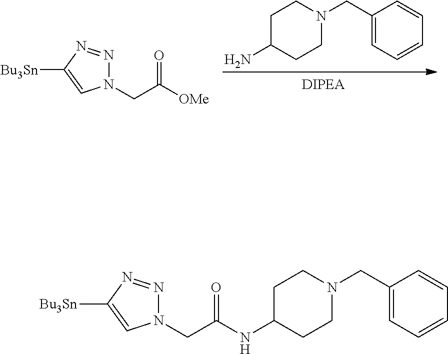

To a solution of the methyl ester (0.12 g, 0.28 mmol) in methanol (1 mL), 4-amino-1-benzylpiperidine (0.16 g, 0.84 mmol) and diisopropylethylamine (0.14 mL, 0.84 mmol) were added. The reaction was heated at 60° C. for 60 h. The reaction mixture was then concentrated by rotary evaporation and purified by silica gel chromatography (50 g SiO₂, dichloromethane/methanol (10:1)). The fractions containing only the desired product were concentrated by rotary evaporation to yield the amide (0.17 g, 64%) as a yellow oil. Melting point, ¹H NMR, ¹³C NMR, IR and HRMS have been completed for the above compound, and the results confirm the structure of the desired compound.

### Preparation of N-(1-benzylpiperidin-4-yl)-2-(4-iodo-1H-1,2,3-triazol-1-yl)acetamide (23)

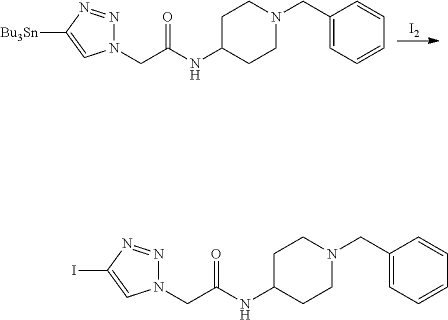

To a solution of the tributyltin triazole (30 mg, 0.05 mmol) in dry THF (3 mL) was added I₂ (15 mg, 0.06 mmol) and the reaction was stirred for 10 minutes at room temperature. Upon completion, the reaction mixture was concentrated by rotary evaporation and purified by silica gel chromatography (˜30 g SiO₂, eluent: dichloromethane/methanol (10:1)). The fractions containing the desired compound were concentrated to yield the iodotriazole (10 mg, 45%) as a white powder. Melting point, ¹H NMR, ¹³C NMR, IR and HRMS have been completed for the above compound, and the results confirm the structure of the desired compound.

### Preparation of N-(2-(piperidin-1-yl)ethyl)-2-(4-(tributylstannyl)-1H-1,2,3-triazol-1-yl)acetamide

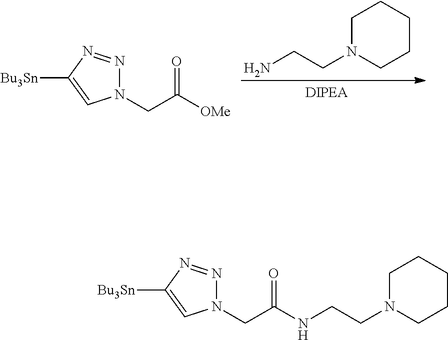

To a solution of the methyl ester (0.28 g, 0.65 mmol) in methanol (1 mL), 2-aminoethylpiperidine (84 mg, 0.65 mmol) and diisopropylethylamine (0.32 mL, 1.9 mmol) were added. The reaction was left to stir at 60° C. overnight. The reaction mixture was then concentrated by rotary evaporation and purified by silica gel chromatography (40 g SiO₂, dichloromethane/methanol (10:1)). The fractions containing only the desired product were concentrated by rotary evaporation to yield the title compound as a yellow oil (0.26 g, 77%). Melting point, ¹H NMR, ¹³C NMR, IR and HRMS have been completed for the above compound, and the results confirm the structure of the desired compound.

### Preparation of 2-(4-iodo-1H-1,2,3-triazol-1-yl)-N-(2-(piperidin-1-yl)ethyl)acetamide

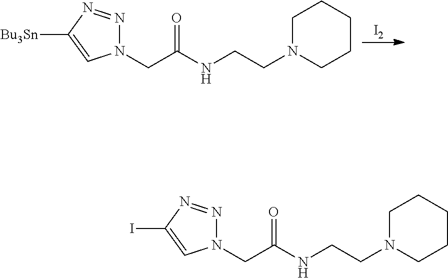

To a solution of the tributyltin triazole (29 mg, 0.055 mmol) in dry THF (3 mL) was added I₂ (15 mg, 0.06 mmol) and the reaction was stirred for 10 min at room temperature. Upon completion, the reaction mixture was concentrated by rotary evaporation and purified by silica gel chromatography (˜30 g SiO₂, eluent: dichloromethane/methanol (10:1)). The fractions containing the desired compound were concentrated to yield 1 mg (5%) of the iodotriazole as a white powder. Melting point, ¹H NMR, ¹³C NMR, IR and HRMS have been completed for the above compound, and the results confirm the structure of the desired compound.

### Preparation of ¹²³I labelled 2-(4-iodo-1H-1,2,3-triazol-1-yl)-N-(2-(piperidin-1-yl)ethyl)acetamide

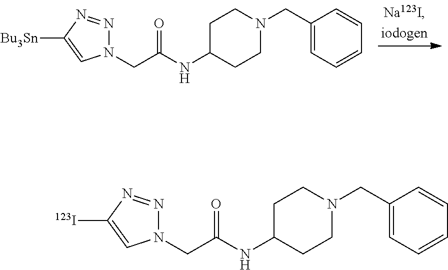

A solution of the tributylstannylated triazole (100 μg) in CHCl₃ (100 μL) and AcOH (5 μL) was added to a 1.5 mL Eppendorf tube. To the reaction mixture was added iodogen (25 μg in 25 μL of CHCl₃) and [Na¹²³I] (7 μL, 2.18 mCi) and the mixture shaken for 10 min. To this was added 0.1 M sodium metabisulphate (100 μL) and the mixture was agitated for 1 min. The aqueous layer was purified by HPLC using a gradient method of H₂O+0.1% trifluoroacetic acid, and ACN+0.1% trifluoroacetic acid. RCY 46%, RCP>99%.

### Preparation of ¹²⁵I labelled 2-(4-iodo-1H-1,2,3-triazol-1-yl)-N-(2-(piperidin-1-yl)ethyl)acetamide

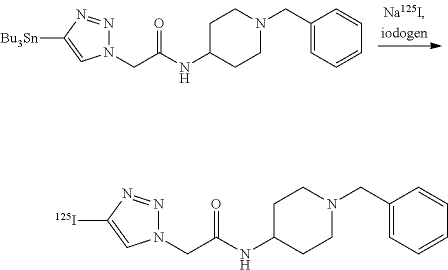

A solution of the tributylstannylated triazole (100 μg) in CHCl₃ (100 μL) and AcOH (5 μL) was added to a 1.5 mL Eppendorf tube. To the reaction mixture was added iodogen (25 μg in 25 μL of CHCl₃) and [Na¹²⁵I] (10 μL, 321 μCi) and the mixture shaken for 10 min. To this was added 0.1 M sodium metabisulphate (100 μL) and the mixture was agitated for 1 min. The aqueous layer was purified by HPLC using a gradient method of H₂O+0.1% trifluoroacetic acid, and ACN+0.1% trifluoroacetic acid. RCY 51%, RCP>99%.

### Formulation for Biodistribution Studies

The HPLC fraction of ¹²⁵I radiolabelled 2-(4-iodo-1H-1,2,3-triazol-1-yl)-N-(2-(piperidin-1-yl)ethyl)acetamide (˜1 mL) was concentrated and reconstituted in 20 nM phosphate buffer solution (1.8 mL, pH 7.4).

### Log P Value Determination

100 μL of phosphate buffer solution containing ¹²⁵I radiolabelled 2-(4-iodo-1H-1,2,3-triazol-1-yl)-N-(2-(piperidin-1-yl)ethyl)acetamide (7 μCi) was added to an equal volume mixture of n-octanol and 20 nM phosphate buffer (pH 7.4). The samples were vortexed for 20 min and centrifuged at 6000 rpm for 30 min, and then aliquots were removed from both the aqueous phosphate buffer and the organic n-octanol layers and counted separately in a γ counter (Wallac Wizard 1470 automatic γ counter). The extractions were done in three sets that contained three extractions in each set for a total number of nine data points. The partition coefficients were then calculated using the equation P=(activity concentration in n-octanol)/(activity concentration in aqueous layer). The log P value was determined to be 0.70±0.19.

Mouse Biodistribution of ¹²⁵I radiolabelled 2-(4-iodo-1H-1,2,3-triazol-1-yl)-N-(2-(piperidin-1-yl)ethyl)acetamide

Biodistribution of ¹²⁵I radiolabelled 2-(4-iodo-1H-1,2,3-triazol-1-yl)-N-(2-(piperidin-1-yl)ethyl)acetamide was performed using female C57Bl/6 mice (n=3 per time point at t=4, 24 h). The mice were administered 0.22 mBq of ¹²⁵1-TAAG-ABP (100 μL in PBS) via tail vein injection. Animals were anesthetized with 3% Isoflurane and euthanized by cervical dislocation. Blood, heart, lungs, liver, gall bladder, spleen, kidneys, adrenals, stomach, small intestine, large intestine and caecum, tumour, adipose, thyroid/trachea, eyes, bone, skeletal muscle, brain, urine and bladder, and tail were weighed and counted in an automated γ counter.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE SPECIFICATION

¹ a) Rösch, F.; Baum, R. P. Generator-based PET radiopharmaceuticals for molecular imaging of tumours: on the way to THERANOSTICS. Dalton Trans. 2011, 40, 6104-6111. b) Agdeppa, E. D.; Spilker, M. E. A review of imaging agent development. The AAPS Journal 2009, 11, 286-299. c) Lammers, T.; Aime, S.; Hennink, W. E.; Storm, G.; Kiessling, F. Theranostic Nanomedicines. Acc. Chem. Res. 2011, 44, 1029-1038. d) Lopci, E.; Chiti, A.; Castellani, M. R.; Pepe, G.; Antunovic, L.; Fanti, S.; Bombardieri, E. Matched pairs dosimetry: ¹²⁴I/¹³¹I metaiodobenzylguanidine and ¹²⁴I/¹³¹I and ⁸⁶Y/⁹⁰Y antibodies. Eur. J. Nucl. Med. Mol. Imaging. 2011, 38 (Suppl 1), S28-S40.
² a) Garg, P. K.; Slade, S. K.; Harrison, C. L.; Zalutsky, M. R. Labeling proteins using aryl iodide acylation agents: influence of meta vs para substitution on in vivo stability. Nucl. Med. Biol. 1989, 16, 669-673. b) Shankar, S.; Vaidyanathan, G.; Affleck, D.; Welsh, P. C.; Zalutsky, M. R. N-Succinimidyl 3-[¹³¹I]Iodo-4-phosphonomethylbenzoate ([¹³¹I]SIPMB), a Negatively Charged Substituent-Bearing Acylation Agent for the Radioiodination of Peptides and mAbs. Bioconjugate Chem. 2003, 14, 331-341. c) Vaidyanathan, G.; Zalutsky, M. R.; DeGrado, T. R.; Iodopyridine-for-Iodobenzene Substitution for Use with Low Molecular Weight Radiopharmaceuticals: Application to m-Iodobenzylguanidine. Bioconjugate Chem. 1998, 9, 758-764. d) Garg, S.; Garg, P. K.; Zalutsky, M. R.; N-Succinimidyl 5-(trialkylstannyl)-3-pyridinecarboxylates: a new class of reagents for protein radioiodination. Bioconjugate Chem. 1991, 2, 50-56. e) Foulon, C. F.; Alston, K. L.; Zalutsky, M. R.; Synthesis and Preliminary Biological Evaluation of (3-Iodobenzoyl)norbiotinamide and ((5-Iodo-3-pyridinyl)carbonyl)norbiotinamide: Two Radioiodinated Biotin Conjugates with Improved Stability. Bioconjugate Chem. 1997, 8, 179-186. f) Reist, C. J.; Garg, P. K.; Alston, K. L.; Bigner, D. D.; Zalutsky, M. R. Radioiodination of internalizing monoclonal antibodies using N-succinimidyl 5-iodo-3-pyridinecarboxylate. Cancer Res. 1996, 56, 4970-4977.
³ Yan, R.; El-Emir, E.; Rajkumar, V.; Robson, M.; Jathoul, A. P.; Pedley, R. B.; Årstad, E. One-Pot Synthesis of an ¹²⁵I-Labeled Trifunctional Reagent for Multiscale Imaging with Optical and Nuclear Techniques. Angew. Chem. Int. Ed. 2011, 50, 6793-6795; Angew. Chem. 2011, 123, 6925-6927.
⁴ a) Hanamoto, T.; Hakoshima, Y.; Egashira, M. Tributyl(3,3,3-trifluoro-1-propynyl)stannane as an efficient reagent for the preparation of various trifluoromethylated heterocyclic compounds. Tetrahedron Lett. 2004, 45, 7573-7576. b) Ito, S.; Hirata, Y.; Nagatomi, Y.; Satoh, A.; Suzuki, G.; Kimura, T.; Satow, A.; Maehara, S.; Hikichi, H.; Hata, M.; Ohta, H.; Kawamoto, H. Discovery and biological profile of isoindolinone derivatives as novel metabotropic glutamate receptor 1 antagonists: A potential treatment for psychotic disorders. Bioorg. Med. Chem. Lett. 2009, 19, 5310-5313. c) Sakamoto, T.; Uchiyama, D.; Kondo, Y.; Yamanaka, H. Synthesis and reaction of 1-phenyl-4-(trimethylstannyl)-1,2,3-triazoles. Heterocycles 1993, 35, 1273-1278. d) Ito, S.; Satoh, A.; Nagatomi, Y.; Hirata, Y.; Suzuki, G.; Kimura, T.; Satow, A.; Maehara, S.; Hikichi, H.; Hata, M.; Kawamoto, H.; Ohta, H. Discovery and biological profile of 4-(1-aryltriazol-4-yl)-tetrahydropyridines as an orally active new class of metabotropic glutamate receptor 1 antagonist. Bioorg. Med. Chem. 2008, 16, 9817-9829. e) Ali, H.; vanLier, J. E. Synthesis of radiopharmaceuticals via organotin intermediates. Synthesis 1996, 4, 423-438.
⁵ a) Donovan, A.; Forbes, J.; Dorff, P.; Schaffer, P.; Babich, J.; Valliant, J. F. A New Strategy for Preparing Molecular Imaging and Therapy Agents Using Fluorine-Rich (Fluorous) Soluble Supports. J. Am. Chem. Soc. 2006, 128, 3536-3537. b) Bejot, R.; Fowler, T.; Carroll, L.; Boldon, S.; Moore, J. E.; Declerck, J.; Gouverneur, V. Fluorous synthesis of ¹⁸F radiotracers with the [¹⁸F]fluoride ion: nucleophilic fluorination as the detagging process. Angew. Chem. Int. Ed. 2009, 48, 586-589; Angew. Chem. 2009, 121, 594-597.
⁶ Nygren, C. L.; Wilson, C. C.; Turner, J. F. C. On the Solid State Structure of 4-Iodobenzoic Acid. J. Phys. Chem. A 2005, 109, 2586-2593.
⁷ a) Li, Y-C.; Qi, C.; Li, S-H.; Zhan, H-J.; Sun, C-H.; Yu, Y-Z.; Pang, S-P. 1,1′-Azobis-1,2,3-triazole: A High-Nitrogen Compound with Stable N8 Structure and Photochromism. J. Am. Chem. Soc., 2010, 132, 12172-12173. b) Domnin, I. N.; Remizova, L. A.; Starova, G. L.; Rominger, F. Synthesis and properties of 5-alkynyl-1,2,3-triazoles. Russ. J. Org. Chem. 2009, 45, 1678-1682.
⁸ Maresca, K. P.; Marquis, J. C.; Hillier, S. M.; Lu, G.; Femia, F. J.; Zimmerman, C. N.; Eckelman, W. C.; Joyal, J. L.; Babich, J. W. Novel Polar Single Amino Acid Chelates for Technetium-99m Tricarbonyl-Based Radiopharmaceuticals with Enhanced Renal Clearance: Application to Octreotide. Bioconjugate Chem. 2010, 21, 1032-1042.
⁹ a) Nan, F.; Bzdega, T.; Pshenichkin, S.; Wroblewski, J. T.; Wroblewska, B.; Neale, J. H.; Kozikowski, A. P. Dual Function Glutamate-Related Ligands: Discovery of a Novel, Potent Inhibitor of Glutamate Carboxypeptidase II Possessing mGluR3Agonist Activity. J. Med. Chem. 2000, 43, 772-774. b) Kozikowski, A. P.; Nan, F.; Conti, P.; Zhang, J. H.; Ramadan, E.; Bzdega, T.; Wroblewska, B.; Neale, J. H.; Pshenichkin, S.; Wroblewski, J. T. Design of Remarkably Simple, Yet Potent Urea-Based Inhibitors of Glutamate Carboxypeptidase II (NAALADase). J. Med. Chem. 2001, 44, 298-301.
¹⁰ a) Maresca, K. P.; Hillier, S. M.; Femia, F. J.; Keith, D.; Barone, C.; Joyal, J. L.; Zimmerman, C. N.; Kozikowski, A. P.; Barrett, J. A.; Eckelman, W. C.; Babich, J. W. A series of halogenated heterodimeric inhibitors of prostate specific membrane antigen (PSMA) as radiolabeled probes for targeting prostate cancer. J. Med. Chem. 2009, 52, 347-357. b) Chen, Y.; Foss, C. A.; Byun, Y.; Nimmagadda, S.; Pullambhatla, M.; Fox, J. J.; Castanares, M.; Lupoid, S. E.; Babich, J. W.; Mease, R. C.; Pomper, M. G. Radiohalogenated Prostate-Specific Membrane Antigen (PSMA)-Based Ureas as Imaging Agents for Prostate Cancer. J. Med. Chem. 2008, 51, 7933-7943. c) Foss, C. A.; Mease, R. C.; Fan, H.; Wang, Y.; Ravert, H. T.; Dannals, R. F.; Olszewski, R. T.; Heston, W. D.; Kozikowski, A. P.; Pomper, M. G. Radiolabeled Small-Molecule Ligands for Prostate-Specific Membrane Antigen: In vivo Imaging in Experimental Models of Prostate Cancer. Clin. Cancer Res. 2005, 11, 4022-4028. d) Hillier, S. M.; Maresca, K. P.; Femia, F. J.; Marquis, J. C.; Foss, C. A.; Nguyen, N.; Zimmerman, C. N.; Barret, J. A.; Eckelman, W. C.; Pomper, M. G.; Joyal, J. L.; Babich, J. W. Preclinical Evaluation of Novel Glutamate-Urea-Lysine Analogues That Target Prostate-Specific Membrane Antigen as Molecular Imaging Pharmaceuticals for Prostate Cancer. Cancer Res. 2009, 69, 6932-6940; e) Hillier, S. M.; Kern, A. M.; Maresca, K. P.; Marquis, J. C.; Eckelman, W. C.; Joyal, J. L.; Babich, J. W. ¹²³I-MIP-1072, a small-molecule inhibitor of prostate-specific membrane antigen, is effective at monitoring tumor response to taxane therapy. J. Nucl. Med. 2011, 52, 1087-1093.
¹¹ a) Stanbury, J. B. Deiodination of the iodinated amino acids. Ann. N.Y. Acad. Sci. 1960, 86 II, 417-439. b) Won, C. M. Kinetics of degradation of levothyroxine in aqueous solution and in solid state. Pharm. Res. 1992, 9, 131-137. c) Dumont, F.; Slegers, G. Synthesis and in vivo evaluation of 7-chloro-5-[¹²³I]iodo-4-oxo-1,4-dihydroquinoline-2-carboxylic acid. Appl. Radiat. Isot. 1997, 48, 1173-1177. d) Al Hussainy, R.; Verbeek, J.; van der Born, D.; Braker, A. H.; Leysen, J. E.; Knol, R. J.; Booij, J.; Herscheid, J. D. M. Design, Synthesis, Radiolabeling, and in Vitro and in Vivo Evaluation of Bridgehead Iodinated Analogues of N-{2-[4-(2-Methoxyphenyl)piperazin-1-yl]ethyl}-N-(pyridin-2-yl)cyclohexanecarboxamide (WAY-100635) as Potential SPECT Ligands for the 5-HT_{1A} Receptor. J. Med. Chem. 2011, 54, 3480-3491. e) Choi, T. H.; Ahn, S. H.; Kwon, H. C.; Choi, C. W.; Awh, O. D.; Lim, S. M. In vivo comparison of IVDU and IVFRU in HSV1-TK gene expressing tumor bearing rats. Appl. Radiat. Isot. 2004, 60, 15-21.
¹² a) Marik, J.; Sutcliffe, J. L. Click for PET: rapid preparation of [¹⁸F]fluoropeptides using CuI catalyzed 1,3-dipolar cycloaddition. Tetrahedron Lett. 2006, 47, 6681-6684. b) Li, Z-B.; Wu, Z.; Chen, K.; Chin, F. T.; Chen, X. Click Chemistry for ¹⁸F-Labeling of RGD Peptides and microPET Imaging of Tumor Integrin αvβ3 Expression. Bioconjugate Chem. 2007, 18, 1987-1994. c) Glaser, M.; Årstad, E. “Click Labeling” with 2-[¹⁸F]Fluoroethylazide for Positron Emission Tomography. Bioconjugate Chem. 2007, 18, 989-993.
¹³ Kiyokawa, K.; Tachikake, N.; Yasuda, M.; Baba, A. Angew. Chem. Int. Ed. 2011, 50, 10393-10396.
¹⁴ Sheehan, J. C.; Bolhofer, W. A. J. Am. Chem. Soc., 1950, 72 (6), 2786-2788.
¹⁵ Postma, R.; Schroder, F. H. Eur. J. Cancer, 2005, 41, 825-833.

TABLE 1
Tissue distribution of ¹²³I-TAAG-PSMA in
NCr nude mice bearing LNCaP xenografts

| | Time and % ID/g post injection | | |
|---|---|---|---|
| Organ | 1 hr | 1 hr + block | 24 h |
| Blood | 0.81 ± 0.07 | 0.32 ± 0.06 | 0.01 ± 0.00 |
| Heart | 0.85 ± 0.29 | 0.15 ± 0.04 | 0.01 ± 0.00 |
| Lung | 2.58 ± 0.46 | 0.36 ± 0.08 | 0.01 ± 0.00 |
| Liver | 2.32 ± 0.28 | 2.25 ± 0.29 | 0.03 ± 0.01 |
| Gall Bladder | 1.28 ± 0.26 | 1.32 ± 0.53 | 0.19 ± 0.08 |
| Spleen | 4.98 ± 1.31 | 0.13 ± 0.03 | 0.03 ± 0.00 |
| Kidneys | 137.54 ± 11.43 | 9.57 ± 5.17 | 0.99 ± 0.27 |
| Adrenals | 8.45 ± 2.13 | 3.34 ± 1.49 | 0.33 ± 0.07 |
| Stomach | 0.90 ± 0.07 | 0.49 ± 0.07 | 0.07 ± 0.03 |
| Small Intestine | 0.64 ± 0.13 | 0.30 ± 0.09 | 0.02 ± 0.01 |
| Large Intestine + | 0.27 ± 0.06 | 0.12 ± 0.03 | 0.04 ± 0.01 |
| Caecum | | | |
| LNCap Tumour | 22.44 ± 5.89 | 1.31 ± 0.64 | 15.31 ± 4.23 |
| Prostate | 1.89 ± 0.62 | 3.85 ± 2.91 | 0.09 ± 0.03 |
| Testes | 0.69 ± 0.10 | 1.16 ± 1.05 | 0.01 ± 0.00 |
| Esophagus | 1.3 ± 0.26 | 0.32 ± 0.04 | 0.07 ± 0.01 |
| Thyroid/Trachea | 1.46 ± 0.3 | 0.62 ± 0.24 | 9.36 ± 1.05 |
| Bone | 0.41 ± 0.07 | 0.26 ± 0.02 | 0.04 ± 0.01 |
| Skeletal Muscle | 0.33 ± 0.04 | 0.19 ± 0.03 | 0.01 ± 0.00 |
| Brain | 0.06 ± 0.01 | 0.05 ± 0.02 | 0.03 ± 0.00 |
| Bladder | 1.78 ± 0.23 | 10.78 ± 4.16 | 0.08 ± 0.02 |
| Urine | 43.99 ± 5.54 | 486.70 ± 163.60 | 1.04 ± 0.39 |
| Salivary Glands | 2.97 ± 0.95 | 0.52 ± 0.12 | 0.05 ± 0.01 |
| Adipose | 1.73 ± 0.26 | 0.35 ± 0.21 | 0.02 ± 0.01 |

Note:
Data are % ID/g, expressed as mean ± SEM.

TABLE 2
Tumor to tissue ratios for ¹²³I-TAAG-PSMA
in NCr nude mice bearing LNCaP xenografts.

| Ratio/Time (h) | 1 | 1 + block | 24 |
|---|---|---|---|
| Tumour/blood | 28.22 ± 7.12 | 4.35 ± 1.99 | 1867 ± 499.72 |
| Tumour/skeletal muscle | 78.06 ± 30.62 | 8.26 ± 5.11 | 1531 ± 422.74 |

Note:
Data are ratios based on % ID/g and expressed as mean ± SEM.

TABLE 3
Crystal data and structure refinement for ¹²⁷I-TAAG

| Identification code | itaag2_0m | |
|---|---|---|
| Empirical formula | C₄H₄IN₃O₂ | |
| Formula weight | 253.00 | |
| Temperature | 100(2) K | |
| Wavelength | 0.71073 Å | |
| Crystal system | Monoclinic | |
| Space group | P2(1)/n | |
| Unit cell dimensions | a = 12.7128(13) Å | α = 90° |
| | b = 4.4805(5) Å | β = 111.252(2)° |
| | c = 13.5180(13) Å | γ = 90° |

| | |
|---|---|
| Volume | 717.62(13) Å³ |
| Z | 4 |
| Density (calculated) | 2.342 Mg/m³ |
| Absorption coefficient | 4.405 mm⁻¹ |
| F(000) | 472 |
| Crystal size | 0.32 × 0.169 × 0.108 mm³ |
| θ range for data collection | 1.88 to 30.52° |
| Index ranges | −17 ≦ h ≦ 18, −6 ≦ k ≦ 3, −19 ≦ l ≦ 19 |
| Reflections collected | 12388 |
| Independent reflections | 2195 [R(int) = 0.0196] |
| Completeness to θ = 30.52° | 99.5% |
| Absorption correction | Numerical |
| Max. and min. transmission | 0.6789 and 0.4131 |
| Refinement method | Full-matrix least-squares on F² |
| Data/restraints/parameters | 2195/0/107 |
| Goodness-of-fit on F² | 1.045 |
| Final R indices [I > 2σ(I)] | R1 = 0.0187, wR2 = 0.0432 |
| R indices (all data) | R1 = 0.0222, wR2 = 0.0447 |
| Largest diff. peak and hole | 0.642 and −0.801 e.Å⁻³ |

TABLE 4
Bond lengths [Å] and angles [°] for ¹²⁷I-TAAG

| | | | |
|---|---|---|---|
| I(1)—C(1) | 2.0617(18) | C(2)—N(1)—C(3) | 128.15(17) |
| O(1)—C(4) | 1.312(2) | N(3)—C(1)—C(2) | 108.55(16) |
| O(1)—H(1) | 0.82(4) | N(3)—C(1)—I(1) | 123.35(13) |
| N(1)—N(2) | 1.337(2) | C(2)—C(1)—I(1) | 128.07(15) |
| N(1)—C(2) | 1.346(2) | N(3)—N(2)—N(1) | 106.50(16) |
| N(1)—C(3) | 1.448(2) | N(1)—C(2)—C(1) | 104.15(17) |
| C(1)—N(3) | 1.352(2) | N(1)—C(2)—H(2A) | 121.1(16) |
| C(1)—C(2) | 1.372(3) | C(1)—C(2)—H(2A) | 134.7(16) |
| O(2)—C(4) | 1.212(2) | N(2)—N(3)—C(1) | 109.05(15) |
| N(2)—N(3) | 1.323(2) | N(1)—C(3)—C(4) | 111.00(16) |
| C(2)—H(2A) | 0.88(3) | N(1)—C(3)—H(3B) | 108.2(17) |
| C(3)—C(4) | 1.520(3) | C(4)—C(3)—H(3B) | 109.0(18) |
| C(3)—H(3B) | 0.94(3) | N(1)—C(3)—H(3A) | 108.2(15) |
| C(3)—H(3A) | 0.97(3) | C(4)—C(3)—H(3A) | 108.6(16) |
| | | H(3B)—C(3)—H(3A) | 112(2) |
| C(4)—O(1)—H(1) | 108(3) | O(2)—C(4)—O(1) | 125.54(18) |
| N(2)—N(1)—C(2) | 111.74(15) | O(2)—C(4)—C(3) | 123.30(16) |
| N(2)—N(1)—C(3) | 119.84(16) | O(1)—C(4)—C(3) | 111.13(17) |

TABLE 5
Torsion angles [°] for ¹²⁷I-TAAG

| | | | |
|---|---|---|---|
| C(2)—N(1)—N(2)—N(3) | −0.3(2) | C(2)—C(1)—N(3)—N(2) | 0.0(2) |
| C(3)—N(1)—N(2)—N(3) | −174.83(17) | I(1)—C(1)—N(3)—N(2) | 177.96(14) |
| N(2)—N(1)—C(2)—C(1) | 0.2(2) | N(2)—N(1)—C(3)—C(4) | 95.7(2) |
| C(3)—N(1)—C(2)—C(1) | 174.25(18) | C(2)—N(1)—C(3)—C(4) | −77.9(2) |
| N(3)—C(1)—C(2)—N(1) | −0.1(2) | N(1)—C(3)—C(4)—O(2) | −14.4(3) |
| I(1)—C(1)—C(2)—N(1) | −178.00(14) | N(1)—C(3)—C(4)—O(1) | 167.34(17) |
| N(1)—N(2)—N(3)—C(1) | 0.2(2) | | |

TABLE 6

| | August 2^{[1]} | August 23 | August 24 | September 17 |
|---|---|---|---|---|
| IC50 (nM) | 124.1 | 174.5 | 94.7 | 115.6 |
| 95% CI | 94.8-162.6 | 118.7-256.4 | 64.6-138.8 | 82.4-162.3 |
| Std Error of | 1.14 | 1.20 | 1.20 | 1.18 |
| curve fit | | | | |
| R² | 0.9885 | 0.9779 | 0.9772 | 0.9819 |

^{[1]}completed with first batch of ¹²⁵I-amino-TAAG-PSMA.

TABLE 7

| | Combined Aggregate | Combined Experimental |
|---|---|---|
| | Data (GraphPad) | Averaged data (Excel) |
| IC50 (nM) | 122.5 | 127.2 |
| 95% CI | 95.7-156.8 | 90.1-180 |
| Std Error | 1.13 (of curve fit) | 33.8 (std dev between experiments) |
| R² | 0.9534 | — |

1. A method of preparing a radiohalogenated compound of Formula I: 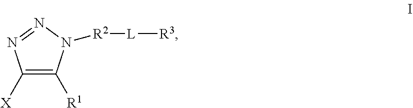
wherein
R¹ is selected from:
(i) H;
(ii) cyano:
(iii) OR⁴;
(iv) NR⁴R⁵;
(v) substituted or unsubstituted C₁₋₆alkyl;
(vi) substituted or unsubstituted C₂₋₆alkenyl;
(vii) substituted or unsubstituted C₃₋₈cycloalkyl;
(viii) substituted or unsubstituted C₃₋₈cycloalkenyl;
(ix) substituted or unsubstituted C₂₋₈heterocycloalkyl;
(x) substituted or unsubstituted C₆₋₁₄aryl; and
(xi) substituted or unsubstituted heteroaryl,
wherein the substituents for C₁₋₆alkyl, C₂₋₆alkenyl, C₃₋₈cycloalkyl, C₃₋₈cycloalkenyl, C₂₋₈heterocycloalkyl, C₆₋₁₄aryl and heteroaryl are selected from F, Cl, Br, I, cyano, oxo, nitro, OR⁴, NR⁴R⁵, C(O)OR⁴, C(O)N⁴R⁵, C₃₋₈cycloalkyl, C₂₋₈heterocycloalkyl, C₆₋₁₀aryl and an immunogenic moiety;
R² is C₁₋₄alkylene;
R₃ is H, C₁₋₄alkyl or a targeting vector;
R⁴ and R⁵ are each independently selected from H, PG, C₁₋₈alkyl, C₂₋₆alkenyl, C₃₋₈cycloalkyl, C₃₋₈cycloalkenyl, C₆₋₁₀aryl, a targeting vector, a fluorophore and an immunogenic moiety; or R⁴ and R⁵ together form PG;
L is an amide linkage or an ester linkage; and
X is a radioisotope of a halogen,
comprising reacting a compound of Formula II: 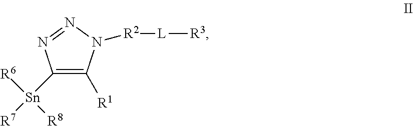
wherein
L, R¹, R² and R³ are as defined for the compound of Formula I; and
R⁶, R⁷ and R⁸ are each independently C₁₋₁₀alkyl or C₁₋₁₀alkyl substituted with one or more F;
with a radiohalogenating agent under conditions to obtain the compound of Formula I.

2. The method of claim 1, wherein the radiohalogenated compound of Formula I is a radiohalogenated compound of Formula I(a): 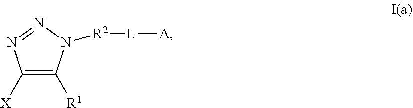
wherein
R¹, R², L and X are as defined for the compound of Formula I of claim 1; and
A is a targeting vector,
and the method comprises reacting a compound of Formula II(a): 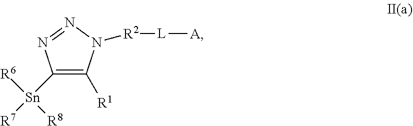
wherein
R¹, R², L and A are as defined for the compound of Formula I(a); and
R⁶, R⁷ and R⁸ are each independently C₁₋₁₀alkyl or C₁₋₁₀alkyl substituted with one or more F,
with a radiohalogenating agent under conditions to obtain the compound of Formula I(a).

3. The method of claim 2, wherein the method further comprises preparing the compound of Formula II(a) by steps comprising:
(a) reacting a compound of Formula III: 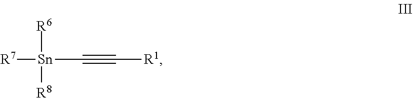
wherein
R¹ is as defined for the compound of Formula I in claim 1; and
R⁶, R⁷ and R⁸ are each independently C₁₋₁₀alkyl or C₁₋₁₀alkyl substituted with one or more F,
with a compound of Formula IV: 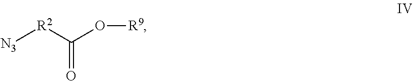
wherein
R² is C₁₋₄alkylene; and
R⁹ is H, C₁₋₄alkyl or an activating group,
under conditions to obtain a compound of Formula V: 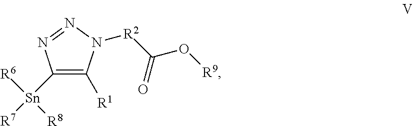
wherein
R¹ is as defined in the compound of Formula I of claim 1;
R² is C₁₋₄alkylene;
R⁶, R⁷ and R⁸ are each independently C₁₋₁₀alkyl or C₁₋₁₀alkyl substituted with one or more F; and
R⁹ is H, C₁₋₄alkyl or an activating group; and
(b) reacting the compound of Formula V with a compound of Formula VI:
H—R¹⁰-A VI,
wherein
R¹⁰ is O or NH; and
A is a targeting vector,
under conditions to obtain the compound of Formula II(a).

4. The method of claim 2, wherein the method further comprises preparing the compound of Formula II(a) by steps comprising:
(a) reacting a compound of Formula VI:
H—R¹⁰-A VI,
wherein
R¹⁰ is O or NH; and
A is a targeting vector,
with a compound of Formula IV: 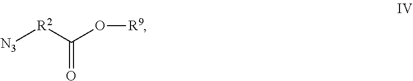
wherein
R² is C₁₋₄alkylene; and
R⁹ is H, C₁₋₄alkyl or an activating group,
under conditions to obtain a compound of Formula VII: 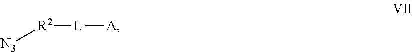
wherein
R² is C₁₋₄alkylene;
L is —C(O)O— or —C(O)NH—; and
A is a targeting vector; and
(b) reacting the compound of Formula VII with a compound of Formula III: 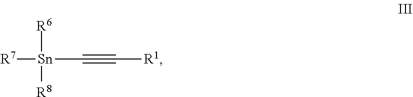
wherein
R¹ is as defined for the compound of Formula I of claim 1; and
R⁶, R⁷ and R⁸ are each independently C₁₋₁₀alkyl or C₁₋₁₀alkyl substituted with one or more F,
under conditions to obtain the compound of Formula II(a).

5. The method of claim 1, wherein the targeting vector targets cancer.

6. The method of claim 5, wherein the cancer is prostate cancer or melanoma.

7. The method of claim 1, wherein L and A together have the structure:

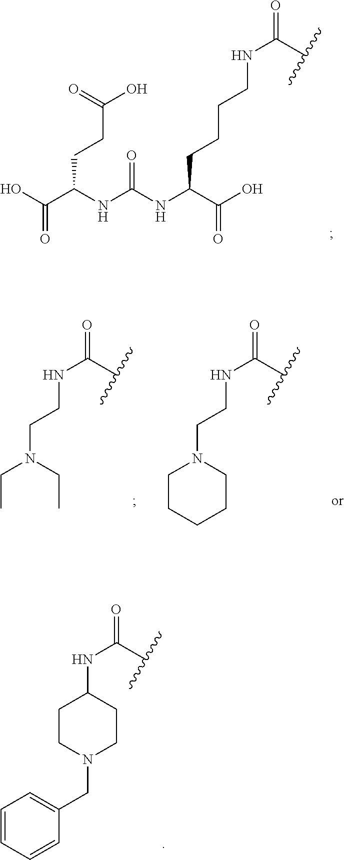 [SMILES: *C(=O)NCCCC[C@H](NC(=O)N[C@@H](CCC(=O)O)C(=O)O)C(=O)O ; *C(=O)NCCN(CC)CC ; *C(=O)NCCN1CCCCC1 ; *C(=O)NC1CCN(Cc2ccccc2)CC1]

8. The method of claim 1, wherein R¹ is selected from H, —CH₂NH₂, —CH₂NH-2,4-dinitrophenyl and phenyl.

9. The method of claim 1, wherein R¹ is H.

10. The method of claim 1, wherein R¹ is —CH₂NH-2,4-dinitrophenyl.

11. The method of claim 1, wherein R² is —CH₂—.

12. The method of claim 1, wherein R⁶, R⁷ and R⁸ are all n-Bu or are all (CH₂)₂(CF₂)₅CF₃.

13. The method of claim 3 or 4, wherein R⁹ is CH₃.

14. The method of claim 1, wherein the radiohalogenating agent is a radioiodinating agent.

15. The method of claim 14, wherein the radioiodinating agent comprises I₂ or NaI, wherein I is a radioisotope of iodine.

16. The method of claim 15, wherein the radioisotope of iodine is ¹²³I, ¹²⁴I, ¹²⁵I or ¹³¹I.

17. A radiohalogenated compound of Formula I: 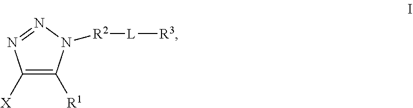
wherein
R¹ is selected from:
(i) H;
(ii) cyano;
(iii) OR⁴;
(iv) NR⁴R⁵;
(v) substituted or unsubstituted C₁₋₆alkyl;
(vi) substituted or unsubstituted C₂₋₆alkenyl;
(vii) substituted or unsubstituted C₃₋₈cycloalkyl;
(viii) substituted or unsubstituted C₃₋₈cycloalkenyl;
(ix) substituted or unsubstituted C₂₋₈heterocycloalkyl;
(x) substituted or unsubstituted C₆₋₁₄aryl; and
(xi) substituted or unsubstituted heteroaryl,
wherein the substituents for C₁₋₆alkyl, C₂₋₆alkenyl, C₃₋₈cycloalkyl, C₃₋₈cycloalkenyl, C₂₋₈heterocycloalkyl, C₆₋₁₄aryl and heteroaryl are selected from F, Cl, Br, I, cyano, oxo, nitro, OR⁴, NR⁴R⁵, C(O)OR⁴, C(O)N⁴R⁵, C₃₋₈cycloalkyl, C₂₋₈heterocycloalkyl, C₆₋₁₀aryl and an immunogenic moiety;
R² is C₁₋₄alkylene;
R³ is H, C₁₋₄alkyl or a targeting vector;
R⁴ and R⁵ are each independently selected from H, PG, C₁₋₆alkyl, C₂₋₆alkenyl, C₃₋₈cycloalkyl, C₃₋₈cycloalkenyl, C₆₋₁₀aryl, a targeting vector, a fluorophore and an immunogenic moiety; or R⁴ and R⁵ together form PG;
L is an amide linkage or an ester linkage; and
X is a radioisotope of a halogen.

18. A compound of Formula II: 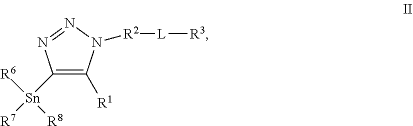
wherein
R¹ is selected from:
(i) H;
(ii) cyano;
(iii) OR⁴;
(iv) NR⁴R⁵;
(v) substituted or unsubstituted C₁₋₆alkyl;
(vi) substituted or unsubstituted C₂₋₆alkenyl;
(vii) substituted or unsubstituted C₃₋₈cycloalkyl;
(viii) substituted or unsubstituted C₃₋₈cycloalkenyl;
(ix) substituted or unsubstituted C₂₋₈heterocycloalkyl;
(x) substituted or unsubstituted C₆₋₁₄aryl; and
(xi) substituted or unsubstituted heteroaryl,
wherein the substituents for C₁₋₆alkyl, C₂₋₆alkenyl, C₃₋₈cycloalkyl, C₃₋₈cycloalkenyl, C₂₋₈heterocycloalkyl, C₆₋₁₄aryl and heteroaryl are selected from F, Cl, Br, I, cyano, oxo, nitro, OR⁴, NR⁴R⁵, C(O)OR⁴, C(O)N⁴R⁵, C₁₋₄alkyl, C₃₋₈cycloalkyl, C₂₋₈heterocycloalkyl, C₆₋₁₀aryl and an immunogenic moiety;
R² is C₁₋₄alkylene;
R⁴ and R⁵ are each independently selected from H, PG, C₁₋₆alkyl, C₂₋₆alkenyl, C₃₋₈cycloalkyl, C₃₋₈cycloalkenyl, C₆₋₁₀aryl, a targeting vector, a fluorophore and an immunogenic moiety; or R⁴ and R⁵ together form PG;
L is an amide linkage or an ester linkage;
R⁶, R⁷ and R⁸ are each independently C₁₋₁₀alkyl or C₁₋₁₀alkyl substituted with one or more F; and
R³ is H, C₁₋₄alkyl or a targeting vector.

19. A composition comprising a compound of claim 18 and a carrier.

20. A use of a compound of Formula II: 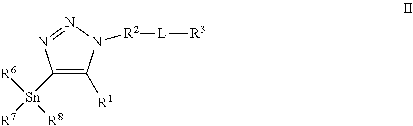
as defined in claim 18 for the preparation of a radiohalogenated compound.
